(19) United States
(12) Patent Application Publication
(10) Pub. No.: US 2023/0346779 A1
(43) Pub. Date: Nov. 2, 2023

(54) DOSAGE FORM COMPOSITIONS COMPRISING AN INHIBITOR OF BTK AND MUTANTS THEREOF

(71) Applicant: Newave Pharmaceutical Inc., Pleasanton, CA (US)

(72) Inventor: Yi Chen, Pleasanton, CA (US)

(21) Appl. No.: 18/020,980

(22) PCT Filed: Aug. 13, 2021
(86) PCT No.: PCT/US2021/045867

§ 371 Date: Feb. 13, 2023

Related U.S. Application Data
(60) Provisional application No. 63/066,105, filed on Aug. 14, 2020.

(30) Foreign Application Priority Data
Aug. 20, 2020 (WO) PCT/US2020/047196

(51) Int. Cl. A61K 31/4985; A61K 9/20; A61K 31/497

(52) U.S. Cl. CPC A61K 31/4985; A61K 9/2013; A61K 9/2018; A61K 9/2054; A61K 31/497

(57) ABSTRACT

Provided herewith are pharmaceutical tablet compositions comprising an organic acid (such as fumaric acid) and a compound of Formula (I), or an N-oxide thereof, solvate, polymorph, tautomer, stereoisomer, an isotopic form, or a prodrug of said compound of Formula (I) or N-oxide thereof: wherein the compound of Formula (I) is an inhibitor of Bruton's tyrosine kinase.

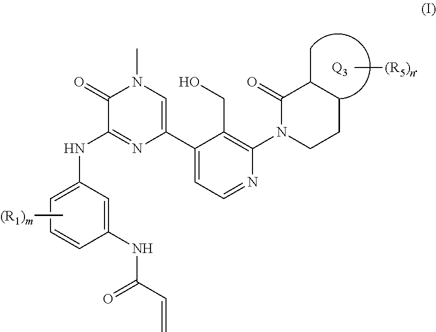

### REFERENCE TO RELATED APPLICATION

This application claims priority to U.S. Provisional Patent Application No. 63/066,105, filed on Aug. 14, 2020, and International Patent Application No. PCT/US2020/047196, filed on Aug. 20, 2020, the entire contents of each of the above-referenced applications are incorporated herein by reference.

### BACKGROUND OF THE INVENTION

Bruton tyrosine kinase (Btk) is a Tec family non-receptor protein kinase, expressed in most hematopoietic cells such as B cells, mast cells, and macrophages but not in T cells, natural killer cells, and plasma cells [Smith, C. I. et al. Journal of Immunology (1994), 152 (2), 557-65]. Btk is a crucial part of the BCR and FcR signaling pathway, and the targeted inhibition of Btk is a novel approach for treating many different human diseases such as B-cell malignancies, autoimmune disease, and inflammatory disorders [Uckun, Fatih M. et al, Anti-Cancer Agents in Medicinal Chemistry (2007), Shinohara et al, Cell 132 (2008) pp 794-806; Pan, Zhengying, Drug News & Perspectives (2008), 21 (7); 7 (6), 624-632; Gilfillan et al, Immunological Reviews 288 (2009) pp 149-169; Davis et al, Nature, 463 (2010) pp 88-94].

Covalent Bruton's tyrosine kinase (BTK) inhibitors including ibrutinib and acalabrutinib have transformed the treatment landscape of several BTK dependent B-cell malignancies, including chronic lymphocytic leukemia, Waldenstrom's macroglobulinemia, mantle cell lymphoma and marginal zone lymphoma. Despite impressive clinical response of ibrutinib in B-cell malignancies, cases of primary and secondary resistance have emerged with poor outcomes and limited treatment options. The majority of CLL patients who become resistant to irreversible BTK inhibitors such as ibrutinib develop the BTK-C481S mutation. It was reported that 80% of patients relapsing CLL will have the C481S mutation [Maddocks K J, et al. JAMA Oncol. 2015; 1:80-87]. Another research group in the Ohio State University reported in Journal of Clinical Oncology [Vol 35, number 13, 2017, page 1437] that at year four, roughly 20% of patients on ibrutinib clinically progressed. Of these patients who relapsed, 85% had acquired the C481S mutation. Additionally, these mutations were detected, on average, over nine months before a relapse.

International Patent Application Nos. PCT/US2019/018139 (WO2019/161152) and PCT/US2020/019478 disclose a highly novel class of BTK inhibitors that can not only irreversibly inhibit wild type BTK but also reversibly inhibit C481S mutant BTK. The molecular weight of these reported compounds is quite high (typically more than 700 g/mol). Unfortunately, the aqueous solubility of some of these compounds in free base form can be quite low. In addition, although the corresponding salt forms of some of these compounds exhibit increased solubility, some of the salts unfortunately are not sufficiently stable and therefore may not be suitable for further formulation development. As such, the formulation of at least some of these compounds may face a significant challenge in assuring acceptable oral bioavailability, which largely dependents on solubility and/or stability in the aqueous medium of the gastrointestinal tract. The challenge becomes even greater when considering the need to provide an adequate drug loading in the formulation, so that a therapeutically effective dose can be administered in an acceptably small volume of formulated product.

### SUMMARY OF THE INVENTION

The present invention is partly based on the discovery that a physical mixture of an organic acid (e.g., fumaric acid) and a BTK inhibitor as disclosed herein in free base form would not only have satisfactory pharmacokinetics (PK) profiles (see examples 3 and 4), but also desired stability (see example 2), as compared to the corresponding BTK inhibitors in free base form alone or in the corresponding pharmaceutically acceptable salt form.

Accordingly, the present invention relates to a tablet composition comprising an organic acid and a compound of Formula (I), or an N-oxide thereof, solvate, polymorph, tautomer, stereoisomer, an isotopic form, or a prodrug of said compound of Formula (I) or N-oxide thereof:

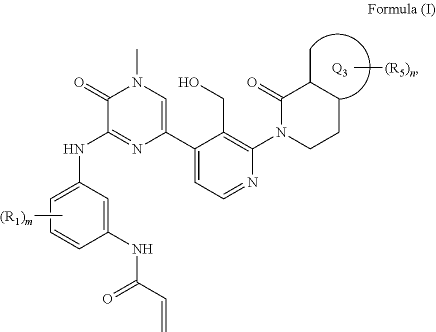

as defined in any one of the embodiments described herein.

In another aspect, the present invention relates to a method of treating a neoplastic disease, particularly the B-cell malignancy including but not limited to B-cell lymphoma, lymphoma (including Hodgkin's and non-Hodgkin's lymphoma), hairy cell lymphoma, small lymphocytic lymphoma (SLL), mantle cell lymphoma (MCL), and diffuse large B-cell lymphoma (DLBCL), multiple myeloma, chronic and acute myelogenous leukemia and chronic and acute lymphocytic leukemia, by administering to a subject in need thereof an effective amount of one or more of the compounds, modifications, and/or salts, and compositions thereof described above.

Autoimmune and/or inflammatory diseases that can be affected using compounds and compositions according to the invention include, but are not limited to: psoriasis, allergy, Crohn's disease, irritable bowel syndrome, Sjogren's disease, tissue graft rejection, and hyperacute rejection of transplanted organs, asthma, systemic lupus erythematosus (and associated glomerulonephritis), dermatomyositis, multiple sclerosis, scleroderma, vasculitis (ANCA-associated and other vasculitides), autoimmune hemolytic and thrombocytopenic states, Goodpasture's syndrome (and associated glomerulonephritis and pulmonary hemorrhage), atherosclerosis, rheumatoid arthritis, chronic Idiopathic thrombocytopenic purpura (ITP), Addison's disease, Parkinson's disease, Alzheimer's disease, diabetes, septic shock, and myasthenia gravis.

The details of one or more embodiments of the invention are set forth in the description below. Other features, objects, and advantages of the invention will be apparent from the description and from the claims. It should be understood that all embodiments/features of the invention (compounds, pharmaceutical compositions, methods of make/use, etc) described herein, including any specific features described in the examples and original claims, can combine with one another unless not applicable or explicitly disclaimed.

### DETAILED DESCRIPTION OF THE INVENTION

### Compounds

The compounds used in the tablet compositions disclosed herein are BTK inhibitors, for examples, the BTK inhibitors disclosed in International Application Nos. PCT/US2019/018139 and PCT/US2020/019478, both of which are incorporated herein by reference.

In one embodiment, the compound used in the tablet compositions is a compound of Formula (I), or an N-oxide thereof, solvate, polymorph, tautomer, stereoisomer, an isotopic form, or a prodrug of said compound of Formula (I) or N-oxide thereof:

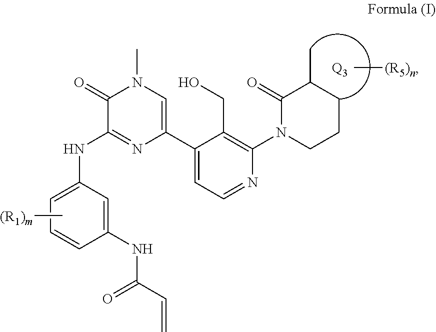

wherein
Q₃ is a 5-membered heteroaryl;
each of R₁ and R₅, independently, is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, aryl, heteroaryl is optionally substituted with one or more R_{d};
two of R₁ groups, taken together with the atom to which they are attached, may optionally form a cycloalkyl or heterocycloalkyl optionally substituted with one or more R_{d};
two of R₅ groups, taken together with the atom to which they are attached, may optionally form a cycloalkyl, heterocycloalkyl, aryl, or heteroaryl optionally substituted with one or more R_{d};
each of Rₐ, R_{b}, R_{c} and R_{d}, independently, is H, D, alkyl, spiroalkyl, alkenyl, alkynyl, halo, cyano, amine, nitro, hydroxy, ═O, —P(O)R_{b}R_{c}, -alkyl-P(O)R_{b}R_{c}, —S(O)(═N(R_{b}))R_{c}, —N═S(O)R_{b}R_{c}, ═NR_{b}, C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally substituted with one or more Rₑ;
each of Rₑ, independently, is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;
two of R_{d} groups, taken together with the atom to which they are attached, may optionally form a cycloalkyl or heterocycloalkyl optionally substituted with one or more Rₑ; and
each of m and n, independently, is 0, 1, 2, 3, or 4.

In another embodiment, the compound used in the tablet compositions is represented by Formula (II):

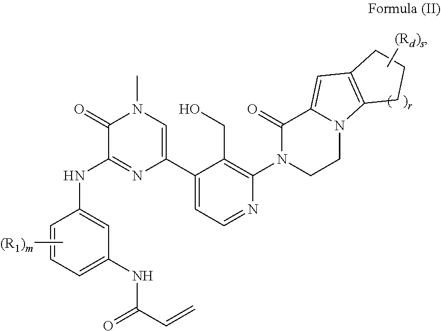

wherein r, and s, each independently, is 0, 1, 2, 3, or 4.

In another embodiment, the compound used in the tablet compositions is represented by Formula (III):

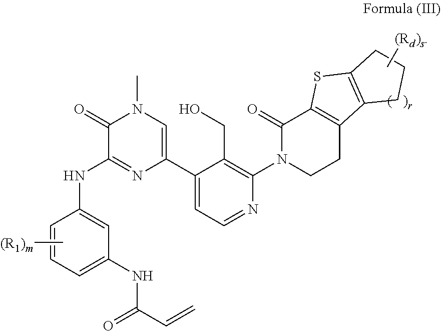

In a specific embodiment, the compound used in the tablet compositions is selected from the group consisting of
(S)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(2-methyl-4-(oxetan-3-yl)piperazin-1-yl)phenyl)acrylamide,
(S)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(2-methyl-4-(tetrahydro-2H-pyran-4-yl)piperazin-1-yl)phenyl)acrylamide,
(R)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(4-(oxetan-3-yl)-2-(trifluoromethyl)piperazin-1-yl)phenyl)acrylamide,
(S)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(4-(oxetan-3-yl)-2-(trifluoromethyl)piperazin-1-yl)phenyl)acrylamide,
(R)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(4-(tetrahydro-2H-pyran-4-yl)-2-(trifluoromethyl)piperazin-1-yl)phenyl)acrylamide,
(S)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(4-(tetrahydro-2H-pyran-4-yl)-2-(trifluoromethyl)piperazin-1-yl)phenyl)acrylamide,
(S)—N-(5-((6-(3-(hydroxymethyl)-2-(1-oxo-3,4,5,6,7,8-hexahydrobenzo[4,5]thieno[2,3-c]pyridin-2(1H)-yl)pyridin-4-yl)-4-methyl-3-oxo-3,4-dihydropyrazin-2-yl)amino)-2-(2-methyl-4-(oxetan-3-yl)piperazin-1-yl)phenyl)acrylamide,
(S)—N-(5-((6-(3-(hydroxymethyl)-2-(1-oxo-3,4,5,6,7,8-hexahydrobenzo[4,5]thieno[2,3-c]pyridin-2(1H)-yl)pyridin-4-yl)-4-methyl-3-oxo-3,4-dihydropyrazin-2-yl)amino)-2-(2-methyl-4-(tetrahydro-2H-pyran-4-yl)piperazin-1-yl)phenyl)acrylamide,
(R)—N-(5-((6-(3-(hydroxymethyl)-2-(1-oxo-3,4,5,6,7,8-hexahydrobenzo[4,5]thieno[2,3-c]pyridin-2(1H)-yl)pyridin-4-yl)-4-methyl-3-oxo-3,4-dihydropyrazin-2-yl)amino)-2-(4-(oxetan-3-yl)-2-(trifluoromethyl)piperazin-1-yl)phenyl)acrylamide,
(S)—N-(5-((6-(3-(hydroxymethyl)-2-(1-oxo-3,4,5,6,7,8-hexahydrobenzo[4,5]thieno[2,3-c]pyridin-2(1H)-yl)pyridin-4-yl)-4-methyl-3-oxo-3,4-dihydropyrazin-2-yl)amino)-2-(4-(oxetan-3-yl)-2-(trifluoromethyl)piperazin-1-yl)phenyl)acrylamide,
(R)—N-(5-((6-(3-(hydroxymethyl)-2-(1-oxo-3,4,5,6,7,8-hexahydrobenzo[4,5]thieno[2,3-c]pyridin-2(1H)-yl)pyridin-4-yl)-4-methyl-3-oxo-3,4-dihydropyrazin-2-yl)amino)-2-(4-(tetrahydro-2H-pyran-4-yl)-2-(trifluoromethyl)piperazin-1-yl)phenyl)acrylamide,
(S)—N-(5-((6-(3-(hydroxymethyl)-2-(1-oxo-3,4,5,6,7,8-hexahydrobenzo[4,5]thieno[2,3-c]pyridin-2(1H)-yl)pyridin-4-yl)-4-methyl-3-oxo-3,4-dihydropyrazin-2-yl)amino)-2-(4-(tetrahydro-2H-pyran-4-yl)-2-(trifluoromethyl)piperazin-1-yl)phenyl)acrylamide.
(S)—N-(2-(4-(4,4-difluorocyclohexyl)-2-methylpiperazin-1-yl)-5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)acrylamide,
N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-((S)-4-((2R,6R)-2,6-dimethyltetrahydro-2H-pyran-4-yl)-2-methylpiperazin-1-yl)phenyl)acrylamide,
N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-((S)-4-((2S,6S)-2,6-dimethyltetrahydro-2H-pyran-4-yl)-2-methylpiperazin-1-yl)phenyl)acrylamide,
N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-((2S)-4-((2R,6S)-2,6-dimethyltetrahydro-2H-pyran-4-yl)-2-methylpiperazin-1-yl)phenyl)acrylamide,
N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-((S)-4-((2R,4s,6S)-2,6-dimethyltetrahydro-2H-pyran-4-yl)-2-methylpiperazin-1-yl)phenyl)acrylamide,
N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-((S)-2-methyl-4-((2S,4S)-2-methyltetrahydro-2H-pyran-4-yl)piperazin-1-yl)phenyl)acrylamide,
N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-((S)-2-methyl-4-((2S,4R)-2-methyltetrahydro-2H-pyran-4-yl)piperazin-1-yl)phenyl)acrylamide,
(S)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(2-methyl-4-(1,4-dithiaspiro[4.5]decan-8-yl)piperazin-1-yl)phenyl)acrylamide,
N-(2-((2S)-4-(2-oxabicyclo[2.2.2]octan-5-yl)-2-methylpiperazin-1-yl)-5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)acrylamide,
N-(2-((2S)-4-((1S,4R)-2-oxabicyclo[2.2.1]heptan-5-yl)-2-methylpiperazin-1-yl)-5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)acrylamide,
N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(2,2-dimethyl-4-(oxetan-3-yl)piperazin-1-yl)phenyl)acrylamide,

### Chemical Synthesis

Description of the synthesis of representative compounds is given below. Other compounds of Formula (I) can be prepared by substantially analogous methods disclosed in International Application Nos. PCT/US2019/018139 and PCT/US2020/019478, as will be clear to one of skill in the art. Where NMR data are presented, ¹H spectra were obtained on XL400 (400 MHz) and are reported as ppm down field from Me₄Si with number of protons, multiplicities, and coupling constants in Hertz indicated parenthetically. Where HPLC data are presented, analyses were performed using an Agilent 1100 system. Where LC/MS data are presented, analyses were performed using an Applied Biosystems API-100 mass spectrometer and Shimadzu SCL-10A LC column:

### Compound 1: Preparation of (S)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(2-methyl-4-(oxetan-3-yl)piperazin-1-yl)phenyl)acrylamide

Into a 1000-mL round-bottom flask purged and maintained with an inert atmosphere of nitrogen, was placed 4-fluoro-3-nitroaniline (50 g, 320.28 mmol, 1.00 equiv), CH₃CN (500 mL), NMM (64.7 g, 639.64 mmol, 2.00 equiv), Cbz-Cl (87.4 g, 512.34 mmol, 1.60 equiv). The resulting solution was stirred overnight at room temperature. The resulting mixture was concentrated under vacuum. The residue was applied onto a silica gel column with ethyl acetate/petroleum ether (1:1). This resulted in 45 g (48%) of benzyl N-(4-fluoro-3-nitrophenyl)carbamate as a yellow solid. LC-MS: (ES, m/z): [M+H]⁺=291, ¹H-NMR: (300 MHz, CDCl₃, ppm): δ8.15 (m, 1H), 7.65 (m, 1H), 7.42-7.32 (m, 5H), 7.22 (m, 1H), 6.80 (s, 2H), 5.22 (s, 2H).

Into a 250-mL round-bottom flask purged and maintained with an inert atmosphere of nitrogen, was placed a solution of benzyl N-(4-fluoro-3-nitrophenyl)carbamate (10 g, 34.45 mmol, 1.00 equiv) in DMSO (100 mL), tert-butyl (3S)-3-methylpiperazine-1-carboxylate (7.58 g, 37.85 mmol), DIEA (6.67 g, 51.61 mmol, 1.50 equiv). The resulting solution was stirred overnight at 110° C. in an oil bath. The resulting solution was diluted with of water. The resulting solution was extracted with of ethyl acetate and the organic layers combined and concentrated under vacuum. The residue was applied onto a silica gel column with ethyl acetate/petroleum ether (1:1). This resulted in 10 g (62%) of tert-butyl (3S)-4-(4-[[(benzyloxy)carbonyl]amino]-2-nitrophenyl)-3-methylpiperazine-1-carboxylate as brown oil. LC-MS: (ES, m/z): [M+H]⁺=471. ¹H-NMR: (300 MHz, CDCl₃, ppm): δ7.86 (s, 1H), 7.60 (m, 1H), 7.44-7.31 (m, 7H), 5.21 (s, 2H), 3.90 (t, J=11.4 Hz, 2H), 3.21-3.02 (m, 3H), 2.79-2.72 (m, 2H), 1.49 (s, 9H), 0.80 (d, J=6.3 Hz, 3H).

Into a 250-mL round-bottom flask purged and maintained with an inert atmosphere of nitrogen, was placed a solution of tert-butyl (3S)-4-(4-[[(benzyloxy)carbonyl]amino]-2-nitrophenyl)-3-methylpiperazine-1-carboxylate (12.5 g, 26.57 mmol, 1.00 equiv) in dioxane (100 mL), hydrogen chloride dioxane (25 mL). The resulting solution was stirred for 30 min at room temperature. The resulting mixture was concentrated under vacuum. This resulted in 12.5 g (crude) of benzyl N-[4-[(2S)-2-methylpiperazin-1-yl]-3-nitrophenyl]carbamate as brown oil. LC-MS: (ES, m/z): 371[M+H]⁺.

Into a 250-mL round-bottom flask purged and maintained with an inert atmosphere of nitrogen, was placed a solution of benzyl N-[4-[(2S)-2-methylpiperazin-1-yl]-3-nitrophenyl]carbamate (12.5 g, 33.75 mmol, 1.00 equiv) in ethanol (100 ml), oxetan-3-one (2.2 g, 30.53 mmol, 1.20 equiv), NaBH₃CN (1.67 g, 26.58 mmol, 1.00 equiv). The resulting solution was stirred for 2 h at room temperature. The resulting mixture was concentrated under vacuum. The residue was applied onto a silica gel column with ethyl acetate/petroleum ether (1:1). This resulted in 5 g (35%) of benzyl N-[4-[(2S)-2-methyl-4-(oxetan-3-yl)piperazin-1-yl]-3-nitrophenyl]carbamate as brown oil. LC-MS: (ES, m/z): 427[M+H]^{+ 1}H-NMR (300 MHz, CD₃OD, ppm): δ7.86 (s, 1H), 7.60 (m, 1H), 7.48-7.31 (m, 6H), 5.21 (s, 2H), 4.75-4.55 (m, 5H), 3.55 (m, 1H), 3.26-3.10 (m, 2H), 2.97-2.72 (m, 3H), 2.30-2.11 (m, 3H), 1.80 (t, J=4.7 Hz, 1H), 1.49 (s, 9H), 0.80 (d, J=6.3 Hz, 3H).

Into a 100-mL round-bottom flask purged and maintained with an inert atmosphere of nitrogen, was placed a solution of benzyl N-[4-[(2S)-2-methyl-4-(oxetan-3-yl)piperazin-1-yl]-3-nitrophenyl]carbamate (5.0 g, 11.72 mmol, 1.00 equiv) in ethanol (50 ml), AcOH (7.0 g, 116.57 mmol, 10.00 equiv). This was followed by the addition of dust Zn (4.6 g, 6.00 equiv). The resulting solution was stirred for 1 h at room temperature. The solids were filtrated out. The resulting mixture was concentrated under vacuum and applied on a silica gel column. This resulted in 1.0 g (22%) of benzyl N-[3-amino-4-[(2S)-2-methyl-4-(oxetan-3-yl)piperazin-1-yl]phenyl]carbamate as brown oil. LC-MS: (ES, m/z): 397[M+H]⁺. ¹H-NMR (300 MHz, CD₃OD, ppm): δ7.46-7.31 (m, 5H), 7.02 (m, 2H), 6.75 (d, J=8.4, 1H), 5.20 (s, 2H), 4.85-4.64 (m, 4H), 3.67-3.55 (m, 3H), 3.17 (m, 1H), 2.92-2.78 (m, 4H), 2.25 (m, 1H), 1.95 (m, 1H), 0.80 (d, J=6.0 Hz, 3H).

Into a 25-mL round-bottom flask purged and maintained with an inert atmosphere of nitrogen, was placed a solution of benzyl N-[3-amino-4-[(2S)-2-methyl-4-(oxetan-3-yl)piperazin-1-yl]phenyl]carbamate (1.0 g, 2.52 mmol, 1.00 equiv) in tetrahydrofuran (10 mL), NMM (510 mg, 5.04 mmol, 2.00 equiv), (Boc)₂O (820 mg, 3.76 mmol, 1.50 equiv). The resulting solution was stirred overnight at room temperature. The resulting mixture was concentrated under vacuum. The residue was applied onto a silica gel column with ethyl acetate/petroleum ether (1:1). This resulted in 0.9 g (72%) of benzyl N-(3-[[(tert-butoxy)carbonyl]amino]-4-[(2S)-2-methyl-4-(oxetan-3-yl)piperazin-1-yl]phenyl)carbamate as brown oil. LC-MS: (ES, m/z): 497[M+H]⁺

Into a 50-mL round-bottom flask purged and maintained with an inert atmosphere of H₂, was placed a solution of benzyl N-(3-[[(tert-butoxy)carbonyl]amino]-4-[(2S)-2-methyl-4-(oxetan-3-yl)piperazin-1-yl]phenyl)carbamate (900 mg, 1.81 mmol, 1.00 equiv) in methanol (10 mL), Palladium carbon (0.1 g, 0.10 equiv). The resulting solution was stirred for 1 h at room temperature. The solids were filtered out. The resulting mixture was concentrated under vacuum. This resulted in 0.6 g (91%) of tert-butyl N-[5-amino-2-[(2S)-2-methyl-4-(oxetan-3-yl)piperazin-1-yl]phenyl]carbamate as brown oil. LC-MS: (ES, m/z): 363[M+H]^{+ 1}H-NMR-PH-: (300 MHz, CD₃OD, ppm): δ7.46-7.31 (m, 5H), 7.02 (m, 2H), 6.75 (d, J=8.4, 1H), 4.78-4.64 (m, 4H), 3.60 (m, 1H), 3.10-2.70 (m, 5H), 2.22 (m, 1H), 1.95 (m, 1H), 0.77 (d, J=6.0 Hz, 3H).

Into a 50-mL round-bottom flask purged and maintained with an inert atmosphere of nitrogen, was placed a solution of tert-butyl N-[5-amino-2-[(2S)-2-methyl-4-(oxetan-3-yl)piperazin-1-yl]phenyl]carbamate (1.2 g, 3.31 mmol, 1.00 equiv) in IPA (10 mL), 3,5-dibromo-1-methyl-1,2-dihydropyrazin-2-one (980 mg, 3.66 mmol, 1.00 equiv), DIEA (640 mg, 4.95 mmol, 1.50 equiv). The resulting solution was stirred overnight at 80° C. in an oil bath. The resulting mixture was concentrated under vacuum. The residue was applied onto a silica gel column with ethyl acetate/petroleum ether (1:1). This resulted in 1.2 g (66%) of tert-butyl N-[5-[(6-bromo-4-methyl-3-oxo-3,4-dihydropyrazin-2-yl)amino]-2-[(2S)-2-methyl-4-(oxetan-3-yl)piperazin-1-yl]phenyl]carbamate as brown oil. LC-MS: (ES, m/z): 551[M+H]+¹H-NMR: (300 MHz, CDCl₃, ppm): δ8.31 (s, 1H), 8.20 (s, 1H), 7.99 (s, 1H), 7.20 (d, J=8.7, 1H), 6.95 (d, J=8.7, 1H), 6.75 (s, 1H), 4.78-4.64 (m, 5H), 3.60 (m, 1H), 3.20-2.72 (m, 7H), 2.22 (m, 1H), 1.95 (m, 1H), 0.79 (d, J=6.0 Hz, 3H).

Into a 50-mL round-bottom flask purged and maintained with an inert atmosphere of nitrogen, was placed a solution of tert-butyl N-[5-[(6-bromo-4-methyl-3-oxo-3,4-dihydropyrazin-2-yl)amino]-2-[(2S)-2-methyl-4-(oxetan-3-yl)piperazin-1-yl]phenyl]carbamate (600 mg, 1.09 mmol, 1.00 equiv) in dichloromethane (6 ml), trifluoroacetic acid (1.2 mL). The resulting solution was stirred for 1 h at room temperature. The resulting mixture was concentrated under vacuum. This resulted in 500 mg (crude) of 3-([3-amino-4-[(2S)-2-methyl-4-(oxetan-3-yl)piperazin-1-yl]phenyl]amino)-5-bromo-1-methyl-1,2-dihydropyrazin-2-one as brown oil. LC-MS: (ES, m/z): 451[M+H]⁺.

Into a 25-mL round-bottom flask purged and maintained with an inert atmosphere of nitrogen, was placed a solution of 3-([3-amino-4-[(2S)-2-methyl-4-(oxetan-3-yl)piperazin-1-yl]phenyl]amino)-5-bromo-1-methyl-1,2-dihydropyrazin-2-one (500 mg, 1.11 mmol, 1.00 equiv) in dioxane (15 mL)/H₂O (1 mL), (2-[4,4-dimethyl-9-oxo-1,10-diazatricyclo[6.4.0.0\hat{ }[2,6]]dodeca-2(6),7-dien-10-yl]-3-[(oxan-2-yloxy)methyl]pyridin-4-yl)boronic acid (431 mg, 0.98 mmol, 1.10 equiv), Pd(dppf)Cl₂ (50 mg, 0.07 mmol, 0.10 equiv), potassium carbonate (307 mg, 2.22 mmol, 2.00 equiv). The resulting solution was stirred for 1 h at 100° C. in an oil bath. The resulting mixture was concentrated under vacuum, dilute with H₂O and extract With EA. This resulted in 500 mg (59%) of 10-[4-[6-([3-amino-4-[(2S)-2-methyl-4-(oxetan-3-yl)piperazin-1-yl]phenyl]amino)-4-methyl-5-oxo-4,5-dihydropyrazin-2-yl]-3-[(oxan-2-yloxy)methyl]pyridin-2-yl]-4,4-dimethyl-1,10-diazatricyclo[6.4.0.0\hat{ }[2,6]]dodeca-2(6),7-dien-9-one(crude) as brown oil. LC-MS: (ES, m/z): 764[M+H]⁺.

Into a 50-mL round-bottom flask purged and maintained with an inert atmosphere of nitrogen, was placed a solution of 10-[4-[6-([3-amino-4-[(2S)-2-methyl-4-(oxetan-3-yl)piperazin-1-yl]phenyl]amino)-4-methyl-5-oxo-4,5-dihydropyrazin-2-yl]-3-[(oxan-2-yloxy)methyl]pyridin-2-yl]-4,4-dimethyl-1,10-diazatricyclo[6.4.0.0\hat{ }[2,6]]dodeca-2(6),7-dien-9-one (500 mg, 0.65 mmol, 1.00 equiv) in dichloromethane (5 mL), trifluoroacetic acid (1 mL). The resulting solution was stirred for 15 min at 40° C. in an oil bath. The resulting mixture was concentrated under vacuum. The crude product was purified by Prep-HPLC. This resulted in 80 mg (18%) of 10-[4-[6-([3-amino-4-[(2S)-2-methyl-4-(oxetan-3-yl)piperazin-1-yl]phenyl]amino)-4-methyl-5-oxo-4,5-dihydropyrazin-2-yl]-3-(hydroxymethyl)pyridin-2-yl]-4,4-dimethyl-1,10-diazatricyclo[6.4.0.0\hat{ }[2,6]]dodeca-2(6),7-dien-9-one as a brown solid. LC-MS: (ES, m/z): 680[M+H]⁺.

Into a 25-mL round-bottom flask purged and maintained with an inert atmosphere of nitrogen, was placed a solution of 10-[4-[6-([3-amino-4-[(2S)-2-methyl-4-(oxetan-3-yl)piperazin-1-yl]phenyl]amino)-4-methyl-5-oxo-4,5-dihydropyrazin-2-yl]-3-(hydroxymethyl)pyridin-2-yl]-4,4-dimethyl-1,10-diazatricyclo[6.4.0.0\hat{ }[2,6]]dodeca-2(6),7-dien-9-one (80 mg, 0.12 mmol, 1.00 equiv) in CH₃CN (1 mL), prop-2-enoic acid (10 mg, 0.14 mmol, 1.20 equiv), HATU (49.2 mg, 0.13 mmol, 1.10 equiv), NMM (17.7 mg, 0.17 mmol, 1.50 equiv). The resulting solution was stirred for 1 h at room temperature. The crude product was purified by Prep-HPLC. This resulted in 27 mg (31%) of N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxo-3,4-dihydropyrazin-2-yl]amino]-2-[(2S)-2-methyl-4-(oxetan-3-yl)piperazin-1-yl]phenyl)prop-2-enamide as a off-white solid. LC-MS: (ES, m/z): 734[M+H]⁺. ¹H-NMR: (300 MHz, d₆-DMSO, ppm): δ9.25 (s, 1H), 9.19 (s, 1H), 9.11 (s, 1H), 8.49 (d, J=5.1 Hz, 1H), 7.95 (d, J=5.1 Hz, 1H), 7.77 (s, 1H), 7.60 (d, J=8.7, 1H), 7.25 (d, J=8.7, 1H), 6.63-6.57 (m, 2H), 6.30 (m, 1H), 5.80 (d, J=3.9 Hz, 1H), 5.02 (m, 1H), 4.65-4.41 (m, 6H), 4.35-4.15 (m, 3H), 3.85 (m, 1H), 3.60-3.43 (m, 4H), 3.10 (m, 1H), 2.85-2.54 (m, 6H), 2.45 (m, 2H), 2.22 (m, 1H), 1.95 (t, J=6.6 Hz, 1H), 1.25 (s, 6H), 0.76 (d, J=6.0 Hz, 3H).

### Compound 2: Preparation of N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxopyrazin-2-yl]amino]-2-[(2S)-2-methyl-4-(oxan-4-yl)piperazin-1-yl]phenyl)prop-2-enamide

Synthesis of [(3,3-dimethylcyclopent-1-en-1-yl)oxy]trimethylsilane: Into a 10-L 4-necked round-bottom flask purged and maintained with an inert atmosphere of nitrogen, was placed CuCl (20.60 g, 208.083 mmol, 0.05 equiv), LiCl (17.64 g, 416.108 mmol, 0.10 equiv), THF (2.50 L). This was followed by the addition of 2-cyclopenten-1-one, 3-methyl-(400.00 g, 4161.075 mmol, 1.00 equiv) at −5 to 5 degrees C. To this was added TMSCl (474.67 g, 4369.129 mmol, 1.05 equiv) dropwise with stirring at −5 to 5 degrees C. To the mixture was added MeMgCl (1670.00 mL, 14495.069 mmol, 3.48 equiv) dropwise with stirring at −5 to 10 degrees C. The resulting solution was stirred for 2 h at −5 to 10 degrees C. in an ice/salt bath. The reaction was then quenched by the addition of 34 g of MeOH. The resulting solution was diluted with 5 L of NH₄Cl. The solids were filtered out. The resulting solution was extracted with 3×5 L of petroleum ether dried over anhydrous sodium sulfate and concentrated. This resulted in 780 g (crude) of [(3,3-dimethylcyclopent-1-en-1-yl)oxy]trimethylsilane as yellow oil. GC-MS: (ES, m/z): M: 184

Synthesis of 3,3-dimethylcyclopentanone: Into a 20-L 4-necked round-bottom flask, was placed [(3,3-dimethylcyclopent-1-en-1-yl)oxy]trimethylsilane (780.00 g, 4230.990 mmol, 1.00 equiv), DCM (7.8 L), H₂O (30.49 g, 1692.396 mmol, 0.4 equiv). This was followed by the addition of POCl₃ (214.09 g, 1396.251 mmol, 0.33 equiv) dropwise with stirring at 25 to 30 degrees C. The resulting solution was stirred for 0.5 hr at 25 degrees C. This solvent straight used for next step. GC-MS: (ES, m/z): M: 112

Synthesis of 3,3-dimethylcyclopentanon: Into a 20-L 4-necked round-bottom flask, was placed 3,3-dimethylcyclopentan-1-one in DCM (7.80 L). This was followed by the addition of DMF (619 g, 2.0 equiv) dropwise with stirring at 25 degrees C. To this was added POCl₃ (1362 g, 2.1 equiv) dropwise with stirring at 40 degrees C. The resulting solution was stirred for overnight at 40 degrees C. in an oil bath. The reaction was then quenched by the addition of 2000 g of K₃PO₄. The resulting solution was extracted with 3×10 L of dichloromethane dried over anhydrous sodium sulfate and concentrated. This resulted in 530 g (4804.86%) of 2-chloro-4,4-dimethylcyclopent-1-ene-1-carbaldehyde as a brown solid. GC-MS: (ES, m/z): M: 158

Synthesis of 4,4-dimethyl-1,10-diazatricyclo[6.4.0.0\hat{ }[2,6]]dodeca-2(6),7-dien-9-one: Into a 5-L 4-necked round-bottom flask, was placed 2-chloro-4,4-dimethylcyclopent-1-ene-1-carbaldehyde (474.00 g, 2988.085 mmol, 1.00 equiv), DMF (3 L), piperazin-2-one (299.17 g, 2988.084 mmol, 1.00 equiv), DIEA (463.43 g, 3585.703 mmol, 1.2 equiv). The resulting solution was stirred for overnight at 115 degrees C. in an oil bath. The reaction mixture was cooled to room temperature with a water/ice bath. The solids were collected by filtration. The resulting mixture was washed with 3×2 L of H₂O and 3×2 L of PE. The solid was dried in an oven under reduced pressure. This resulted in 230 g (37.68%) of 4,4-dimethyl-1,10-diazatricyclo[6.4.0.0\hat{ }[2,6]]dodeca-2(6),7-dien-9-one as a grey solid. LC-MS: (ES, m/z): M+1: 205

Synthesis of 2-[4,4-dimethyl-9-oxo-1,10-diazatricyclo[6.4.0.0\hat{ }[2,6]]dodeca-2(6),7-dien-10-yl]-4-iodopyridine-3-carbaldehyde: Into a 2-L 4-necked round-bottom flask purged and maintained with an inert atmosphere of nitrogen, was placed 4,4-dimethyl-1,10-diazatricyclo[6.4.0.0\hat{ }[2,6]]dodeca-2(6),7-dien-9-one (38.00 g, 1.00 equiv), THF (500.00 mL). This was followed by the addition of LiHMDS (558.80 mL, 3.00 equiv) dropwise with stirring at 0 degrees C. To this was added 2-fluoro-4-iodopyridine-3-carbaldehyde (93.50 g, 2.00 equiv), in portions at degrees C. The resulting solution was stirred for overnight at room temperature. The reaction was then quenched by the addition of 1 L of water. The pH value of the solution was adjusted to 7 with HCl (2 mol/L). The solids were filtered out. The resulting solution was extracted with 3×1 L of ethyl acetate concentrated. The residue was applied onto a silica gel column and eluted with ethyl acetate/petroleum ether (2:3). The collected fractions were combined and concentrated. This resulted in 12 g of 2-[4,4-dimethyl-9-oxo-1,10-diazatricyclo[6.4.0.0\hat{ }[2,6]]dodeca-2(6),7-dien-10-yl]-4-iodopyridine-3-carbaldehyde as a light yellow solid. LC-MS: (ES, m/z): M+1: 436

Synthesis of tert-butyl (3S)-4-[4-[(6-bromo-4-methyl-3-oxopyrazin-2-yl)amino]-2-nitrophenyl]-3-methylpiperazine-1-carboxylate: Into a 50-mL round-bottom flask, was placed 5-bromo-3-[(4-fluoro-3-nitrophenyl)amino]-1-methylpyrazin-2-one (2.00 g, 5.829 mmol, 1.00 equiv), NMP (20.00 mL), tert-butyl (3S)-3-methylpiperazine-1-carboxylate (1.17 g, 5.842 mmol, 1.00 equiv), DIEA (2.26 g, 17.487 mmol, 3.00 equiv). The resulting solution was stirred for 48 h at 110° C. in an oil bath. The resulting solution was diluted with 100 mL of H₂O. The resulting solution was extracted with 3×50 mL of dichloromethane/methanol (10:1). The resulting mixture was washed with 3×20 ml of NaCl.

The resulting mixture was concentrated. The residue was applied onto a silica gel column with dichloromethane/methanol (10:1). This resulted in 3 g (59.00%) of tert-butyl (3S)-4-[4-[(6-bromo-4-methyl-3-oxopyrazin-2-yl)amino]-2-nitrophenyl]-3-methylpiperazine-1-carboxylate as a brown solid. LC-MS: (ES, m/z): M+1: 523

Synthesis of 5-bromo-1-methyl-3-([4-[(2S)-2-methylpiperazin-1-yl]-3-nitrophenyl]amino)pyrazin-2-one: Into a 100-mL round-bottom flask, was placed tert-butyl (3S)-4-[4-[(6-bromo-4-methyl-3-oxopyrazin-2-yl)amino]-2-nitrophenyl]-3-methylpiperazine-1-carboxylate (3.00 g, 1 equiv, 60%), HCl (2M) in 1,4-dioxane (30.00 mL). The resulting solution was stirred for 13 h at room temperature. The resulting mixture was concentrated. The resulting solution was diluted with 30 mL of H₂O. The pH value of the solution was adjusted to 8 with NH₃—H₂O. The resulting solution was extracted with 3×15 mL of dichloromethane concentrated. The residue was applied onto a silica gel column with dichloromethane/methanol (10:1). This resulted in 700 mg (48.09%) of 5-bromo-1-methyl-3-([4-[(2S)-2-methylpiperazin-1-yl]-3-nitrophenyl]amino)pyrazin-2-one as a red solid. LC-MS: (ES, m/z): M+1: 423

Synthesis of 5-bromo-1-methyl-3-([4-[(2S)-2-methyl-4-(oxan-4-yl)piperazin-1-yl]-3-nitrophenyl]amino)pyrazin-2-one: Into a 50-mL round-bottom flask, was placed 5-bromo-1-methyl-3-([4-[(2S)-2-methylpiperazin-1-yl]-3-nitrophenyl]amino)pyrazin-2-one (250 mg, 0.591 mmol, 1.00 equiv), 4H-pyran-4-one, tetrahydro-(70.96 mg, 0.709 mmol, 1.20 equiv), THF (5 ml), AcOH (5 drops), NaBH(AcO)₃ (250.36 mg, 1.181 mmol, 2.00 equiv). The resulting solution was stirred for 14 h at 30° C. in an oil bath. The resulting mixture was concentrated. The residue was applied onto a silica gel column with dichloromethane/methanol (10:1). This resulted in 220 mg (73.41%) of 5-bromo-1-methyl-3-([4-[(2S)-2-methyl-4-(oxan-4-yl)piperazin-1-yl]-3-nitrophenyl]amino)pyrazin-2-one as a white solid. LC-MS: (ES, m/z): M+1: 507

Synthesis of 4-methyl-6-([4-[(2S)-2-methyl-4-(oxan-4-yl)piperazin-1-yl]-3-nitrophenyl]amino)-5-oxopyrazin-2-ylboronic acid: Into a 25-mL round-bottom flask purged and maintained with an inert atmosphere of nitrogen, was placed 5-bromo-1-methyl-3-([4-[(2S)-2-methyl-4-(oxan-4-yl)piperazin-1-yl]-3-nitrophenyl]amino)pyrazin-2-one (200.00 mg, 0.394 mmol, 1.00 equiv), bis(pinacolato)diboron (200.19 mg, 0.788 mmol, 2.00 equiv), THF (5.00 mL, 0.069 mmol, 0.1 equiv), XPhos Pd G3 (16.68 mg, 0.020 mmol, 0.05 equiv), KOAc (77.37 mg, 0.788 mmol, 2.00 equiv). The resulting solution was stirred for 4 h at 70° C. in an oil bath. The solids were filtered out. This resulted in 150 mg (53.98%) of 4-methyl-6-([4-[(2S)-2-methyl-4-(oxan-4-yl)piperazin-1-yl]-3-nitrophenyl]amino)-5-oxopyrazin-2-ylboronic acid as a black solid. LC-MS: (ES, m/z): M+1: 473

Synthesis of 2-[4,4-dimethyl-9-oxo-1,10-diazatricyclo[6.4.0.0\hat{ }[2,6]]dodeca-2(6),7-dien-10-yl]-4-[4-methyl-6-([4-[(2S)-2-methyl-4-(oxan-4-yl)piperazin-1-yl]-3-nitrophenyl]amino)-5-oxopyrazin-2-yl]pyridine-3-carbaldehyde: Into a 40-mL vial purged and maintained with an inert atmosphere of nitrogen, was placed 2-[4,4-dimethyl-9-oxo-1,10-diazatricyclo[6.4.0.0\hat{ }[2,6]]dodeca-2(6),7-dien-10-yl]-4-iodopyridine-3-carbaldehyde (200.00 mg, 0.459 mmol, 1.00 equiv), 4-methyl-6-([4-[(2S)-2-methyl-4-(oxan-4-yl)piperazin-1-yl]-3-nitrophenyl]amino)-5-oxopyrazin-2-ylboronic acid (651.07 mg, 1.378 mmol, 3.00 equiv), THF (8.00 mL), H2O (2.00 mL), K₃PO₄ (292.60 mg, 1.378 mmol, 3.00 equiv), Pd(dppf)Cl₂ (33.62 mg, 0.046 mmol, 0.10 equiv). The resulting solution was stirred for 2 h at 50 degrees C. in an oil bath. The solids were filtered out. The resulting solution was extracted with 3×10 mL of ethyl acetate concentrated. The residue was applied onto a silica gel column and eluted with dichloromethane/methanol (10:1). The collected fractions were combined and concentrated. This resulted in 130 mg (38.45%) of 2-[4,4-dimethyl-9-oxo-1,10-diazatricyclo[6.4.0.0\hat{ }[2,6]]dodeca-2(6),7-dien-10-yl]-4-[4-methyl-6-([4-[(2S)-2-methyl-4-(oxan-4-yl)piperazin-1-yl]-3-nitrophenyl]amino)-5-oxopyrazin-2-yl]pyridine-3-carbaldehyde as a brown solid. LC-MS: (ES, m/z): M+1: 736

Synthesis of 10-[3-(hydroxymethyl)-4-[4-methyl-6-([4-[(2S)-2-methyl-4-(oxan-4-yl)piperazin-1-yl]-3-nitrophenyl]amino)-5-oxopyrazin-2-yl]pyridin-2-yl]-4,4-dimethyl-1,10-diazatricyclo[6.4.0.0\hat{ }[2,6]]dodeca-2(6),7-dien-9-one: Into a 8-mL vial, was placed 2-[4,4-dimethyl-9-oxo-1,10-diazatricyclo[6.4.0.0\hat{ }[2,6]]dodeca-2(6),7-dien-10-yl]-4-[4-methyl-6-([4-[(2S)-2-methyl-4-(oxan-4-yl)piperazin-1-yl]-3-nitrophenyl]amino)-5-oxopyrazin-2-yl]pyridine-3-carbaldehyde (100.00 mg, 0.136 mmol, 1.00 equiv), THF (2.00 mL), H₂O (200.00 uL), K₂HPO₄ (59.18 mg, 0.340 mmol, 2.50 equiv). This was followed by the addition of NaOH(1M) (300.00 uL, 7.501 mmol, 55.19 equiv) dropwise with stirring at 0 degrees C. To this was added NaBH₄ (5.14 mg, 0.136 mmol, 1.00 equiv), in portions at 0 degrees C. The resulting solution was stirred for 20 min at room temperature. The reaction was then quenched by the addition of 2 mL of water. The resulting solution was extracted with 3×5 mL of ethyl acetate concentrated. The residue was applied onto a silica gel column and eluted with dichloromethane/methanol (10:1). The collected fractions were combined and concentrated. This resulted in 50 mg (49.86%) of 10-[3-(hydroxymethyl)-4-[4-methyl-6-([4-[(2S)-2-methyl-4-(oxan-4-yl)piperazin-1-yl]-3-nitrophenyl]amino)-5-oxopyrazin-2-yl]pyridin-2-yl]-4,4-dimethyl-1,10-diazatricyclo[6.4.0.0\hat{ }[2,6]]dodeca-2(6),7-dien-9-one as a yellow solid. LC-MS: (ES, m/z): M+1: 738

Synthesis of 10-[4-[6-([3-amino-4-[(2S)-2-methyl-4-(oxan-4-yl)piperazin-1-yl]phenyl]amino)-4-methyl-5-oxopyrazin-2-yl]-3-(hydroxymethyl)pyridin-2-yl]-4,4-dimethyl-1,10-diazatricyclo[6.4.0.0\hat{ }[2,6]]dodeca-2(6),7-dien-9-one: Into a 30-mL pressure tank reactor, was placed 10-[3-(hydroxymethyl)-4-[4-methyl-6-([4-[(2S)-2-methyl-4-(oxan-4-yl)piperazin-1-yl]-3-nitrophenyl]amino)-5-oxopyrazin-2-yl]pyridin-2-yl]-4,4-dimethyl-1,10-diazatricyclo[6.4.0.0\hat{ }[2,6]]dodeca-2(6),7-dien-9-one (50.00 mg, 0.068 mmol, 1.00 equiv), THF (5.00 mL, 0.069 mmol, 1.02 equiv), PtO₂ (4.62 mg, 0.020 mmol, 0.30 equiv). To the above H₂(g) was introduced in at room temperature. The resulting solution was stirred for 2 h at room temperature. The solids were filtered out. The resulting mixture was concentrated. This resulted in 30 mg (crude) of 10-[4-[6-([3-amino-4-[(2S)-2-methyl-4-(oxan-4-yl)piperazin-1-yl]phenyl]amino)-4-methyl-5-oxopyrazin-2-yl]-3-(hydroxymethyl)pyridin-2-yl]-4,4-dimethyl-1,10-diazatricyclo[6.4.0.0\hat{ }[2,6]]dodeca-2(6),7-dien-9-one as a yellow solid. LC-MS: (ES, m/z): M+1: 708

Synthesis of N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxopyrazin-2-yl]amino]-2-[(2S)-2-methyl-4-(oxan-4-yl)piperazin-1-yl]phenyl)prop-2-enamide hydrochloride: Into a 8-mL vial, was placed 10-[4-[6-([3-amino-4-[(2S)-2-methyl-4-(oxan-4-yl)piperazin-1-yl]phenyl]amino)-4-methyl-5-oxopyrazin-2-yl]-3-(hydroxymethyl)pyridin-2-yl]-4,4-dimethyl-1,10-diazatricyclo[6.4.0.0\hat{ }[2,6]]dodeca-2(6),7-dien-9-one (20.00 mg, 0.028 mmol, 1.00 equiv), DCM (4.00 mL), DIEA (7.30 mg, 0.057 mmol, 2 equiv). This was followed by the addition of acryloyl chloride (2.56 mg, 0.028 mmol, 1.00 equiv) dropwise with stirring at 0 degrees C. The resulting solution was stirred for 2 h at room temperature. The resulting mixture was concentrated. The crude product was purified by Prep-HPLC with the following conditions: Column, X-bridge RP18; mobile phase, 0.05% FA in water and CH₃CN (45% CH₃CN up to 60% in 5 min); Detector, UV 254 nm. The collected solution was concentrated under vacuum to remove CH₃CN and the resulting solution was dried by lyophilization (added with con.HCl(1 drop)). This resulted in 3.3 mg (14.63%) of N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxopyrazin-2-yl]amino]-2-[(2S)-2-methyl-4-(oxan-4-yl)piperazin-1-yl]phenyl)prop-2-enamide hydrochloride as a light yellow solid. LC-MS: (ES, m/z): M+1-HCl: 762. ¹H NMR ((300 MHz, DMSO-d₆, ppm) δ 10.38 (s, 1H), 9.32 (s, 1H), 9.19 (s, 2H), 8.49 (d, J=5.1 Hz, 1H), 7.95 (d, J=5.1 Hz, 1H), 7.78 (s, 1H), 7.69 (d, J=9.0 Hz, 1H), 7.21 (d, J=8.7 Hz, 1H), 6.75 (dd, J=17.1, 10.2 Hz, 1H), 6.57 (s, 1H), 6.46-6.28 (m, 1H), 5.89-5.77 (m, 1H), 4.54 (d, J=17.1 Hz, 2H), 4.22 (s, 3H), 4.02 (d, J=11.4 Hz, 2H), 3.85 (s, 1H), 3.57 (s, 3H), 3.36 (t, J=11.4 Hz, 2H), 3.16 (d, J=12.9 Hz, 1H), 2.97 (t, J=15.0 Hz, 2H), 2.59 (d, J=4.5 Hz, 3H), 2.44 (s, 2H), 2.09 (s, 2H), 1.77 (s, 2H), 1.23 (s, 6H), 0.80 (d, J=6.0 Hz, 3H).

### Compound 3A and 3B: Preparation of N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxopyrazin-2-yl]amino]-2-[(2R)-4-(oxan-4-yl)-2-(trifluoromethyl)piperazin-1-yl]phenyl)prop-2-enamide(assumed) and N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxopyrazin-2-yl]amino]-2-[(2S)-4-(oxan-4-yl)-2-(trifluoromethyl)piperazin-1-yl]phenyl)prop-2-enamide(assumed)

Synthesis of [(3,3-dimethylcyclopent-1-en-1-yl)oxy]trimethylsilane Into a 20-L 4-necked round-bottom flask purged and maintained with an inert atmosphere of nitrogen, was placed CuCl (49.5 g, 500 mmol, 0.05 equiv), LiCl (42.4 g, 1000 mmol, 0.10 equiv), THF (6 L). This was followed by the addition of 2-cyclopenten-1-one, 3-methyl-(960.00 g, 10 mol, 1.00 equiv) at −5 to 5 degrees C. To this was added TMSCl (1140.3 g, 10.5 mol, 1.05 equiv) dropwise with stirring at −5 to 5 degrees C. To the mixture was added MeMgCl (4000 mL, 12 mol, 1.2 equiv) dropwise with stirring at −5 to 10 degrees C. The resulting solution was stirred for 2 h at −5 to 10 degrees C. in an ice/salt bath. The reaction was then quenched by the addition of 82 g of MeOH. The resulting solution was diluted with 10 L of NH₄Cl. The solids were filtered out. The resulting solution was extracted with 3×10 L of petroleum ether dried over anhydrous sodium sulfate and concentrated. This resulted in 1730 g (crude) of [(3,3-dimethylcyclopent-1-en-1-yl)oxy]trimethylsilane as yellow oil. GC-MS: (ES, m/z): M: 184

Synthesis of 3,3-dimethylcyclopentan-1-one Into a 20-L 4-necked round-bottom flask, was placed [(3,3-dimethylcyclopent-1-en-1-yl)oxy]trimethylsilane (1730.00 g, 9.40 mol, 1.00 equiv), DCM (7.0 L), H₂O (67.69 g, 3.76 mol, 0.4 equiv). This was followed by the addition of POCl₃ (474.71 g, 3.10 mol, 0.33 equiv) dropwise with stirring at 25 to 30 degrees C. The resulting solution was stirred for 0.5 hr at 25 degrees C. This crude solvent straight used for next step.

Synthesis of 2-chloro-4,4-dimethylcyclopent-1-ene-1-carbaldehyde Into a 20-L 4-necked round-bottom flask, was placed previous step solution 3,3-dimethylcyclopentan-1-one in DCM (7.0 L). This was followed by the addition of DMF (1372.4 g, 2.0 equiv) dropwise with stirring at 25 degrees C. To this was added POCl₃ (3020.22 g, 2.1 equiv) dropwise with stirring at 40 degrees C. The resulting solution was stirred for overnight at 40 degrees C. in an oil bath. The reaction was then quenched by the addition of 4000 g of K₃PO₄ in 30 L Water. The resulting solution was extracted with 3×20 L of dichloromethane dried over anhydrous sodium sulfate and concentrated. This resulted in 1700 g (Crude) of 2-chloro-4,4-dimethylcyclopent-1-ene-1-carbaldehyde as a brown solid.

Synthesis of 4,4-dimethyl-1,10-diazatricyclo[6.4.0.0\hat{ }[2,6]]dodeca-2(6),7-dien-9-one Into a 10-L 4-necked round-bottom flask, was placed 2-chloro-4,4-dimethylcyclopent-1-ene-1-carbaldehyde (1700.00 g, 10.759 mol, 1.00 equiv), DMF (6 L), piperazin-2-one (1075.95 g, 10.759 mol, 1.00 equiv), DIEA (1665.49 g, 12.91 mol, 1.2 equiv). The resulting solution was stirred for overnight at 115 degrees C. in an oil bath. The reaction mixture was cooled to room temperature with a water/ice bath. The solids were collected by filtration. The resulting mixture was washed with 3×6 L of H₂O and 3×4 L of PE. The solid was dried in an oven under reduced pressure. This resulted in 720 g (32.81%) of 4,4-dimethyl-1,10-diazatricyclo[6.4.0.0\hat{ }[2,6]]dodeca-2(6),7-dien-9-one as a grey solid. LC-MS: (ES, m/z): M+1: 205

Synthesis of 2,4-dibromopyridine-3-carbaldehyde Into a 10000-mL 4-necked round-bottom flask, was placed 2,4-dibromopyridine (500.00 g, 2.11 mol, 1.00 equiv), THF (5000.00 mL). This was followed by the addition of LDA (2M in hexane, 1.58 L, 1.5 equiv) dropwise with stirring at −78 degrees C. The resulting solution was stirred for 1 h at −78 degrees C. Then add DMF (200 g, 2.74 mol, 1.3 equiv) by dropwise with stirring at −78 degrees C. The resulting solution was stirred for 1 h at −78 degrees C. The reaction was then quenched by the addition of 5000 mL of aq. NH₄Cl/HOAc (1:1). The resulting solution was extracted with 3×5000 mL of ethyl acetate concentrated. The residue was applied onto a silica gel column and eluted with ethyl acetate/petroleum ether (0:1-1:1). This resulted in 450 g (80%) of 2,4-dibromopyridine-3-carbaldehyde as a white solid. LC-MS: (ES, m/z): M+1: 264

Synthesis of (2,4-dibromopyridin-3-yl)methanol Into a 10000-mL 4-necked round-bottom flask, was placed 2,4-dibromopyridine-3-carbaldehyde (450 g, 1.7 mol, 1.00 equiv), EtOH (4500.00 mL). This was followed by the addition of NaBH₄ (65 g, 1.7 mol, 1 equiv), in portions at 0 degrees C. The resulting solution was stirred for 3 h at room temperature. The reaction was then quenched by the addition of 3000 mL of water. The resulting solution was extracted with 3×3000 mL of ethyl acetate concentrated. The residue was applied onto a silica gel column and eluted with ethyl acetate/petroleum ether (1:1). The collected fractions were combined and concentrated. This resulted in 500 g (crude, 90%) of (2,4-dibromopyridin-3-yl)methanol as a light yellow solid. LC-MS: (ES, m/z): M+1: 266.

Synthesis of 2,4-dibromo-3-[(oxan-2-yloxy)methyl]pyridine Into a 10-L 4-necked round-bottom flask, was placed (2,4-dibromopyridin-3-yl)methanol (500 g, 1.89 mol, 1.00 equiv), DCM (5 L), PPTS (47.358 g, 188.68 mmol, 0.10 equiv), DHP (237.73 g, 2.83 mol, 1.50 equiv). The resulting solution was stirred for overnight at 45 degrees C. in an oil bath.

The reaction was then quenched by the addition of 3 L of water. The resulting solution was extracted with 3×5 L of dichloromethane concentrated. The residue was applied onto a silica gel column and eluted with ethyl acetate/petroleum ether (1:1). The collected fractions were combined and concentrated. This resulted in 560 g (97.4%) of 2,4-dibromo-3-[(oxan-2-yloxy)methyl]pyridine as colorless oil. LC-MS: (ES, m/z): M+1: 350

Synthesis of 10-[4-bromo-3-[(oxan-2-yloxy)methyl]pyridin-2-yl]-4,4-dimethyl-1,10-diazatricyclo[6.4.0.0\hat{ }[2,6]]dodeca-2(6),7-dien-9-one Into a 5 L 4-necked round-bottom flask purged and maintained with an inert atmosphere of nitrogen, was placed 2,4-dibromo-3-[(oxan-2-yloxy)methyl]pyridine (200.00 g, 569.739 mmol, 1.00 equiv), DMA (2.60 L), 4,4-dimethyl-1,10-diazatricyclo[6.4.0.0\hat{ }[2,6]]dodeca-2(6),7-dien-9-one (128.02 g, 626.713 mmol, 1.10 equiv), K₂CO₃ (236.22 g, 1709.194 mmol, 3.00 equiv), CuI (65.10 g, 341.843 mmol, 0.60 equiv), 1,10-phenanthroline (61.60 g, 341.832 mmol, 0.60 equiv). The resulting solution was stirred for overnight at 110 degrees C. in an oil bath. The reaction mixture was cooled to room temperature with an ice/salt bath. The solids were filtered out. The resulting solution was extracted with 3×6 L of ethyl acetate concentrated. The residue was applied onto a silica gel column with dichloromethane/methanol (10:1). The collected fractions were combined and concentrated. This resulted in 150 g (92%) and 100 g (33%) of 10-[4-bromo-3-[(oxan-2-yloxy)methyl]pyridin-2-yl]-4,4-dimethyl-1,10-diazatricyclo[6.4.0.0\hat{ }[2,6]]dodeca-2(6),7-dien-9-one as a brown solid. LC-MS: (ES, m/z): M+1: 474

Synthesis of 2-[4,4-dimethyl-9-oxo-1,10-diazatricyclo[6.4.0.0\hat{ }[2,6]]dodeca-2(6),7-dien-10-yl]-3-[(oxan-2-yloxy)methyl]pyridin-4-ylboronic acid Into a 3 L 4-necked round-bottom flask purged and maintained with an inert atmosphere of nitrogen, was placed 10-[4-bromo-3-[(oxan-2-yloxy)methyl]pyridin-2-yl]-4,4-dimethyl-1,10-diazatricyclo [6.4.0.0\hat{ }[2,6]]dodeca-2(6),7-dien-9-one (150 g, 321.3 mmol, 1.00 equiv), dioxane (1.5 L), bis(pinacolato)diboron (201.37 g, 792.8 mmol, 2.50 equiv), KOAc (93.23 g, 951.37 mmol, 3.00 equiv), Pd(dppf)Cl₂ (23.19 g, 31.71 mmol, 0.10 equiv). The resulting solution was stirred for 2 h at 100 degrees C. in an oil bath. The reaction mixture was cooled to room temperature.

The solids were filtered out. The resulting mixture was concentrated under vacuum. Then added CH₃CN (300 mL) to residue, the solids were filtered out. This resulted in 70 g (94%) and 120 g (30%) of 2-[4,4-dimethyl-9-oxo-1,10-diazatricyclo[6.4.0.0\hat{ }[2,6]]dodeca-2(6),7-dien-10-yl]-3-[(oxan-2-yloxy)methyl]pyridin-4-ylboronic acid as brown oil. LC-MS (ES, m/z): M+1: 440

Synthesis of 10-[1-hydroxy-3H-[1,2]oxaborolo[4,3-c]pyridin-4-yl]-4,4-dimethyl-1,10-diazatricyclo[6.4.0.0\hat{ }[2,6]]dodeca-2(6),7-dien-9-one Into a 2 L round-bottom flask, was placed 2-[4,4-dimethyl-9-oxo-1,10-diazatricyclo[6.4.0.0\hat{ }[2,6]]dodeca-2(6),7-dien-10-yl]-3-[(oxan-2-yloxy)methyl]pyridin-4-ylboronic acid (70 g, 148 mmol, 1.00 equiv), dioxane (350 mL), HCl/dioxane (4N, 350 mL). The resulting solution was stirred for 1 h at room temperature. The resulting mixture was concentrated under vacuum. The crude product was purified by re-crystallization from Et₂O. The solids were collected by filtration This resulted in 45 g (95%) and 24 g (33%) of 10-[1-hydroxy-3H-[1,2]oxaborolo[4,3-c]pyridin-4-yl]-4,4-dimethyl-1,10-diazatricyclo[6.4.0.0\hat{ }[2,6]]dodeca-2(6),7-dien-9-one as a light yellow solid. LC-MS: (ES, m/z): M+1: 338

Synthesis of 2-(trifluoromethyl)pyrazine Into a 1-L 3-necked round-bottom flask purged and maintained with an inert atmosphere of nitrogen, was placed 2-iodopyrazine (20.00 g, 97.094 mmol, 1.00 equiv), DMSO (200.00 mL), CuI (3.70 g, 19.428 mmol, 0.20 equiv), 1,10-phenanthroline (3.50 g, 19.419 mmol, 0.2 equiv), KF (16.92 g, 291.239 mmol, 3.00 equiv), B(OMe)₃ (30.27 g, 291.282 mmol, 3.00 equiv), TMSCF₃ (41.42 g, 291.290 mmol, 3.00 equiv). The resulting solution was stirred for 2 hr at 60 degrees C. in an oil bath. The resulting solution was diluted with 1 L of H₂O. The resulting solution was extracted with 3×150 mL of ethyl acetate. The resulting mixture was washed with 1×150 of H₂O. The resulting mixture was washed with 1×100 mL of NaCl. The mixture was dried over anhydrous sodium sulfate and concentrated. The residue was applied onto a silica gel column and purified with ethyl acetate/petroleum ether (1:1). This resulted in crude of 2-(trifluoromethyl)pyrazine in 40 ml EA. Product is volatile. LC-MS: (ES, m/z): 149 [M+H]⁺

Synthesis of 2-(trifluoromethyl)piperazine Into a 1-L pressure tank reactor, was placed 2-(trifluoromethyl)pyrazine (crude in 40 ml EA), MeOH (200 ml), Pd/C (2.00 g). To the above H₂ (g) was introduced in. The resulting solution was stirred for 14 hr at 60 degrees C. in an oil bath. The solids were filtered out. The resulting mixture was concentrated. This resulted in 4.0 g of 2-(trifluoromethyl)piperazine(crude) as a solid. LC-MS: (ES, m/z): 155 [M+H]⁺

Synthesis of tert-butyl 3-(trifluoromethyl)piperazine-1-carboxylate Into a 250-mL round-bottom flask, was placed 2-(trifluoromethyl)piperazine (4.00 g, crude), THF (100.00 mL), Boc₂O (8.50 g, 38.947 mmol, 1.50 equiv). The resulting solution was stirred for 14 hr at room temperature. The resulting mixture was concentrated. The residue was applied onto a silica gel column and purified with ethyl acetate/petroleum ether (1:1). This resulted in 3.2 g (48.50%) of tert-butyl 3-(trifluoromethyl)piperazine-1-carboxylate as a white solid. H-NMR: (300 MHz, Chloroform-d) δ 4.13 (d, J=16.2 Hz, 1H), 3.94-3.74 (m, 1H), 3.24 (dtd, J=10.2, 6.6, 3.0 Hz, 1H), 3.16-2.87 (m, 3H), 2.78 (td, J=12.7, 11.7, 4.8 Hz, 1H), 2.07 (s, 1H), 1.49 (s, 9H).

Synthesis of tert-butyl 4-(4-nitrophenyl)-3-(trifluoromethyl)piperazine-1-carboxylate Into a 250-mL round-bottom flask purged and maintained with an inert atmosphere of nitrogen, was placed tert-butyl 3-(trifluoromethyl)piperazine-1-carboxylate (3.20 g, 12.586 mmol, 1.00 equiv), 4-bromo-1-nitrobenzene (5.08 g, 25.148 mmol, 2.00 equiv), 2G-Ad2n-BuP Pd(0.42 g, 20.629 mmol), Cs₂CO₃ (12.30 g, 37.751 mmol, 3.00 equiv), Toluene (100.00 mL). The resulting solution was stirred for 14 hr at 105 degrees C. The resulting mixture was concentrated. The residue was applied onto a silica gel column and purified with ethyl acetate/petroleum ether (1:3). This resulted in 4 g (84.67%) of tert-butyl 4-(4-nitrophenyl)-3-(trifluoromethyl)piperazine-1-carboxylate as a brown solid. H-NMR: (300 MHz, Chloroform-d) δ 8.29-8.07 (m, 2H), 7.07-6.78 (m, 2H), 4.63-4.24 (m, 3H), 3.74-2.95 (m, 4H), 1.49 (s, 9H).

Synthesis of 1-(4-nitrophenyl)-2-(trifluoromethyl)piperazine Into a 250-mL round-bottom flask, was placed tert-butyl 4-(4-nitrophenyl)-3-(trifluoromethyl)piperazine-1-carboxylate (4.00 g), HCl in 1,4-dioxane (100.00 mL, 2M). The resulting solution was stirred for 14 hr at room temperature. The resulting mixture was concentrated. The resulting solution was diluted with 100 mL of DCM. The resulting mixture was washed with 3×25 ml of NaHCO₃. The resulting mixture was washed with 1×25 mL of NaCl. The mixture was dried over anhydrous sodium sulfate. This resulted in 2.5 g of 1-(4-nitrophenyl)-2-(trifluoromethyl) piperazine as a brown solid.

Synthesis of 1-(4-nitrophenyl)-4-(oxan-4-yl)-2-(trifluoromethyl)piperazine Into a 50-mL round-bottom flask, was placed 1-(4-nitrophenyl)-2-(trifluoromethyl)piperazine (1.50 g, 5.450 mmol, 1.00 equiv), tetrahydro-4H-pyran-4-one (1.09 g, 10.900 mmol, 2.00 equiv), DCE (20.00 mL), HOAc (0.10 mL, 0.002 mmol), NaBH(AcO)₃ (2.89 g, 13.636 mmol, 2.50 equiv). The resulting solution was stirred for 14 hr at room temperature. The resulting mixture was concentrated. The residue was applied onto a silica gel column and eluted with ethyl acetate/petroleum ether (1:3). This resulted in 1.8 g (91.91%) of 1-(4-nitrophenyl)-4-(oxan-4-yl)-2-(trifluoromethyl)piperazine as a brown solid. LC-MS (ES, m/z): 360 [M+H]⁺

Synthesis of 1-(2-bromo-4-nitrophenyl)-4-(oxan-4-yl)-2-(trifluoromethyl)piperazine Into a 50-mL round-bottom flask, was placed 1-(4-nitrophenyl)-4-(oxan-4-yl)-2-(trifluoromethyl)piperazine (1.80 g, 5.009 mmol, 1.00 equiv), TFA (20.00 mL), NBS (1.78 g, 10.018 mmol, 2.00 equiv). The resulting solution was stirred for 4 hr at room temperature. The resulting solution was diluted with 100 mL of DCM. The resulting mixture was washed with 3×20 ml of NaHCO₃. The resulting mixture was washed with 1×20 mL of NaCl. The mixture was dried over anhydrous sodium sulfate. The residue was applied onto a silica gel column and eluted with ethyl acetate/petroleum ether (1:5). This resulted in 1.2 g (54.66%) of 1-(2-bromo-4-nitrophenyl)-4-(oxan-4-yl)-2-(trifluoromethyl)piperazine as a yellow solid. H-NMR (300 MHz, DMSO-d₆) δ 8.41 (d, J=2.7 Hz, 1H), 8.22 (dd, J=9.0, 2.7 Hz, 1H), 7.48 (d, J=9.0 Hz, 1H), 4.62 (d, J=8.7 Hz, 1H), 3.90 (d, J=11.1 Hz, 2H), 3.70 (t, J=11.7 Hz, 1H), 3.34 (s, 1H), 3.24 (s, 1H), 3.09 (d, J=11.7 Hz, 1H), 2.96 (d, J=11.1 Hz, 1H), 2.70 (d, J=13.2 Hz, 1H), 2.38 (t, J=10.8 Hz, 1H), 1.70 (t, J=11.1 Hz, 2H), 1.47 (q, J=13.2, 12.0 Hz, 2H).

Synthesis of tert-butyl N-[5-nitro-2-[4-(oxan-4-yl)-2-(trifluoromethyl)piperazin-1-yl]phenyl]carbamate Into a 50-mL round-bottom flask purged and maintained with an inert atmosphere of nitrogen, was placed 1-(2-bromo-4-nitrophenyl)-4-(oxan-4-yl)-2-(trifluoromethyl)piperazine (1.20 g, 2.738 mmol, 1.00 equiv), BocNH₂ (0.96 g, 8.215 mmol, 3.00 equiv), Toluene (20.00 mL), Xantphos Pd 2G (0.12 g, 0.135 mmol, 0.05 equiv), Cs₂CO₃ (2.68 g, 8.225 mmol, 3.00 equiv). The resulting solution was stirred for 2 hr at 90 degrees C. in an oil bath. The resulting mixture was concentrated. The residue was applied onto a silica gel column and eluted with ethyl acetate/petroleum ether (1:1). This resulted in 1.1 g (84.67%) of tert-butyl N-[5-nitro-2-[4-(oxan-4-yl)-2-(trifluoromethyl)piperazin-1-yl]phenyl]carbamate as a brown solid. LC-MS: (ES, m/z): 475 [M+H]⁺

Synthesis of tert-butyl N-[5-amino-2-[4-(oxan-4-yl)-2-(trifluoromethyl)piperazin-1-yl]phenyl]carbamate Into a 50-mL round-bottom flask, was placed tert-butyl N-[5-nitro-2-[4-(oxan-4-yl)-2-(trifluoromethyl)piperazin-1-yl]phenyl]carbamate (1.10 g, 2.318 mmol, 1.00 equiv), MeOH (20.00 mL, 493.978 mmol, 213.08 equiv), PD/C (0.17 g, 0.452 mmol, 0.19 equiv). To the above H₂ (g, 5 atm) was introduced. The resulting solution was stirred for 14 hr at room temperature. The solids were filtered out. The resulting mixture was concentrated. This resulted in 900 mg (87.34%) of tert-butyl N-[5-amino-2-[4-(oxan-4-yl)-2-(trifluoromethyl)piperazin-1-yl]phenyl]carbamate as a brown solid. LC-MS: (ES, m/z): 445 [M+H]⁺

Synthesis of tert-butyl (5-((6-bromo-4-methyl-3-oxo-3,4-dihydropyrazin-2-yl)amino)-2-(4-(tetrahydro-2H-pyran-4-yl)-2-(trifluoromethyl)piperazin-1-yl)phenyl)carbamate Into a 50-mL round-bottom flask purged and maintained with an inert atmosphere of nitrogen, was placed tert-butyl N-[5-amino-2-[4-(oxan-4-yl)-2-(trifluoromethyl)piperazin-1-yl]phenyl]carbamate (900.00 mg, 2.025 mmol, 1.00 equiv), 3,5-dibromo-1-methylpyrazin-2-one (813.67 mg, 3.037 mmol, 1.50 equiv), Pd-PEPPS™-IPent catalyst (160.50 mg, 0.202 mmol, 0.10 equiv), Cs₂CO₃ (1.98 g, 6.077 mmol, 3.00 equiv), Toluene (15.00 ml). The resulting solution was stirred for 14 hr at 90 degrees C. in an oil bath.

The resulting mixture was concentrated. The residue was applied onto a silica gel column and eluted with ethyl acetate/petroleum ether (1:1). This resulted in 450 mg of tert-butyl (5-((6-bromo-4-methyl-3-oxo-3,4-dihydropyrazin-2-yl)amino)-2-(4-(tetrahydro-2H-pyran-4-yl)-2-(trifluoromethyl)piperazin-1-yl)phenyl)carbamate as a brown solid. H-NMR (300 MHz, DMSO-d₆) δ 9.41 (s, 1H), 8.29 (d, J=2.4 Hz, 1H), 7.97 (s, 1H), 7.62 (dd, J=8.7, 2.4 Hz, 1H), 7.39-7.29 (m, 2H), 3.91 (d, J=12.1 Hz, 3H), 3.44 (s, 3H), 3.11-2.96 (m, 3H), 2.82 (d, J=12.4 Hz, 3H), 2.69-2.57 (m, 2H), 1.76-1.4 (m, 2H), 1.48 (s, 12H).

Synthesis of 3-([3-amino-4-[4-(oxan-4-yl)-2-(trifluoromethyl)piperazin-1-yl]phenyl]amino)-5-bromo-1-methylpyrazin-2-one Into a 25-mL round-bottom flask, was placed tert-butyl N-[5-[(6-bromo-4-methyl-3-oxopyrazin-2-yl)amino]-2-[4-(oxan-4-yl)-2-(trifluoromethyl)piperazin-1-yl]phenyl]carbamate (350.00 mg, 1 equiv), DCM (6.00 mL), TFA (2.00 mL). The resulting solution was stirred for 2 hr at room temperature. The resulting solution was diluted with 10 mL of DCM. The resulting mixture was washed with 3×10 ml of NaHCO₃. The resulting mixture was washed with 1×10 mL of NaCl. The mixture was dried over anhydrous sodium sulfate and concentrated. This resulted in 250 mg of 3-([3-amino-4-[4-(oxan-4-yl)-2-(trifluoromethyl)piperazin-1-yl]phenyl]amino)-5-bromo-1-methylpyrazin-2-one as a brown solid. LC-MS: (ES, m/z): 531 [M+H]⁺

Synthesis of N-[5-[(6-bromo-4-methyl-3-oxopyrazin-2-yl)amino]-2-[4-(oxan-4-yl)-2-(trifluoromethyl)piperazin-1-yl]phenyl]prop-2-enamide Into a 8-mL vial, was placed 3-([3-amino-4-[4-(oxan-4-yl)-2-(trifluoromethyl)piperazin-1-yl]phenyl]amino)-5-bromo-1-methylpyrazin-2-one (240.00 mg, 0.452 mmol, 1.00 equiv), DCM (5.00 mL), TEA (68.55 mg, 0.677 mmol, 1.50 equiv), acryloyl chloride (44.97 mg, 0.497 mmol, 1.10 equiv). The resulting solution was stirred for 1 hr at 0 degrees C. in a water/ice bath. The reaction was then quenched by the addition of 0.1 mL of MeOH. The resulting mixture was concentrated. The residue was applied onto a silica gel column and eluted with dichloromethane/methanol (100:5). This resulted in 220 mg (83.20%) of N-[5-[(6-bromo-4-methyl-3-oxopyrazin-2-yl)amino]-2-[4-(oxan-4-yl)-2-(trifluoromethyl)piperazin-1-yl]phenyl]prop-2-enamide as a brown solid. LC-MS: (ES, m/z): 585 [M+H]⁺

Synthesis of N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxopyrazin-2-yl]amino]-2-[4-(oxan-4-yl)-2-(trifluoromethyl)piperazin-1-yl]phenyl)prop-2-enamide Into a 8-mL vial purged and maintained with an inert atmosphere of nitrogen, was placed N-[5-[(6-bromo-4-methyl-3-oxopyrazin-2-yl)amino]-2-[4-(oxan-4-yl)-2-(trifluoromethyl)piperazin-1-yl]phenyl]prop-2-enamide (80.00 mg, 0.137 mmol, 1.00 equiv), dioxane (3.00 mL), H₂O (0.30 mL), 10-[1-hydroxy-3H-[1,2]oxaborolo[4,3-c]pyridin-4-yl]-4,4-dimethyl-1,10-diazatricyclo[6.4.0.0\hat{ }[2,6]]dodeca-2(6),7-dien-9-one (55.29 mg, 0.164 mmol, 1.20 equiv), Pd(DtBPF)Cl₂ (8.91 mg, 0.014 mmol, 0.10 equiv), K₂CO₃ (56.66 mg, 0.410 mmol, 3.00 equiv). The resulting solution was stirred for 1 hr at 90 degrees C. in an oil bath. The resulting mixture was concentrated. The residue was purified by Prep-TLC with dichloromethane/methanol (100:5). This resulted in 70 mg (62.78%) of N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxopyrazin-2-yl]amino]-2-[4-(oxan-4-yl)-2-(trifluoromethyl)piperazin-1-yl]phenyl)prop-2-enamide as a brown solid. LCMS (ES, m/z): M+1: 816

Synthesis of N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxopyrazin-2-yl]amino]-2-[(2R)-4-(oxan-4-yl)-2-(trifluoromethyl)piperazin-1-yl]phenyl)prop-2-enamide(assumed)N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxopyrazin-2-yl]amino]-2-[4-(oxan-4-yl)-2-(trifluoromethyl)piperazin-1-yl]phenyl)prop-2-enamide was purified by Chiral-Prep-HPLC with the following conditions (SHIMADZU LC-20AT): Column, CHIRALPAK ID-3,4.6*50 MM, 3 um; Mobile Phase A: Ethanol (0.1% DEA), Mobile Phase B: ACN, Flow rate: 1.0 ml/min; Detector: 254 nm. This resulted in 17 mg of N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxopyrazin-2-yl]amino]-2-[(2R)-4-(oxan-4-yl)-2-(trifluoromethyl)piperazin-1-yl]phenyl)prop-2-enamide(assumed, RT=3.2 mi) as a yellow solid. LCMS (ES, m/z): M+1: 816. H-NMR: (300 MHz, Chloroform-d) δ 9.28 (d, J=18.3 Hz, 1H), 8.87 (s, 1H), 8.64 (d, J=5.1 Hz, 1H), 8.39 (s, 1H), 8.19 (s, 2H), 7.51 (s, 1H), 6.86 (s, 1H), 6.44 (d, J=16.8 Hz, 1H), 6.28 (dd, J=16.8, 10.2 Hz, 1H), 5.79 (dd, J=10.2, 1.5 Hz, 1H), 5.18 (d, J=12.6 Hz, 1H), 4.73 (d, J=12.0 Hz, 1H), 4.63-4.32 (m, 2H), 4.27-3.99 (m, 4H), 3.88 (d, J=13.2 Hz, 1H), 3.74-3.69 (m, 4H), 3.44 (t, J=11.6 Hz, 2H), 3.22 (s, 1H), 3.07-2.85 (s, 3H), 2.57 (d, J=18.3 Hz, 6H), 1.91-1.72 (m, 2H), 1.75-1.62 (m, 2H), 1.30 (s, 6H).

Synthesis of N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxopyrazin-2-yl]amino]-2-[(2S)-4-(oxan-4-yl)-2-(trifluoromethyl)piperazin-1-yl]phenyl)prop-2-enamide(assumed) N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxopyrazin-2-yl]amino]-2-[4-(oxan-4-yl)-2-(trifluoromethyl)piperazin-1-yl]phenyl)prop-2-enamide (70.00 mg) was purified by Chiral-Prep-HPLC with the following conditions (SHIMADZU LC-20AT): Column, CHIRALPAK ID-3,4.6*50 MM, 3 um; Mobile Phase A: Ethanol (0.1% DEA), Mobile Phase B: ACN, Flow rate: 1.0 ml/min; Detector: 254 nm. This resulted in 16 mg of N-(5-[[6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)-4-methyl-3-oxopyrazin-2-yl]amino]-2-[(2S)-4-(oxan-4-yl)-2-(trifluoromethyl)piperazin-1-yl]phenyl)prop-2-enamide(assumed, RT=2.0 min) as a white solid. LCMS (ES, m/z): M+1: 816. H-NMR: (300 MHz, Chloroform-d) δ 9.28 (d, J=18.3 Hz, 1H), 8.87 (s, 1H), 8.64 (d, J=5.1 Hz, 1H), 8.39 (s, 1H), 8.19 (s, 2H), 7.51 (s, 1H), 6.86 (s, 1H), 6.44 (d, J=16.8 Hz, 1H), 6.28 (dd, J=16.8, 10.2 Hz, 1H), 5.79 (dd, J=10.2, 1.5 Hz, 1H), 5.18 (d, J=12.6 Hz, 1H), 4.73 (d, J=12.0 Hz, 1H), 4.63-4.32 (m, 2H), 4.27-3.99 (m, 4H), 3.88 (d, J=13.2 Hz, 1H), 3.74-3.69 (m, 4H), 3.44 (t, J=11.6 Hz, 2H), 3.22 (s, 1H), 3.07-2.85 (s, 3H), 2.57 (d, J=18.3 Hz, 6H), 1.91-1.72 (m, 2H), 1.75-1.62 (m, 2H), 1.30 (s, 6H).

### Compound 4, Preparation of N-[5-([6-[3-(hydroxymethyl)-2-[6-oxo-8-thia-5-azatricyclo[7.4.0.0\hat{ }[2,7]]trideca-1(9),2(7)-dien-5-yl]pyridin-4-yl]-4-methyl-3-oxopyrazin-2-yl]amino)-2-[(2S)-2-methyl-4-(oxetan-3-yl)piperazin-1-yl]phenyl]prop-2-enamide

Synthesis of 2, 4-dibromopyridine-3-carbaldehyde: Into a 1000-mL 3-necked round-bottom flask, was placed 2, 4-dibromopyridine (40.00 g, 168.852 mmol, 1.00 equiv), THF (400.00 mL). This was followed by the addition of LDA (2M in hexane, 126.60 mL, 1.50 equiv) dropwise with stirring at −78 degrees C. The resulting solution was stirred for 1 h at −78 degrees C. Then add DMF (16.04 ml, 219.507 mmol, 1.30 equiv) dropwise with stirring at −78 degrees C. The resulting solution was stirred for 0.5 h at −78 degrees C. The reaction was then quenched by the addition of 500 mL of NH₄Cl. The resulting solution was extracted with 3×500 mL of ethyl acetate concentrated. The residue was applied onto a silica gel column and eluted with ethyl acetate/petroleum ether (0:1-1:1). This resulted in 24.4 g (54.55%) of 2, 4-dibromopyridine-3-carbaldehyde as a white solid. LCMS-1 (ES, m/z): M+1: 264

Synthesis of (2, 4-dibromopyridin-3-yl) methanol: Into a 100-mL round-bottom flask, was placed 2, 4-dibromopyridine-3-carbaldehyde (2.00 g, 7.550 mmol, 1.00 equiv), EtOH (30.00 mL). This was followed by the addition of NaBH₄ (285.64 mg, 7.550 mmol, 1 equiv), in portions at 0 degrees C. The resulting solution was stirred for 3 h at room temperature. The reaction was then quenched by the addition of 30 mL of water. The resulting solution was extracted with 3×30 mL of ethyl acetate concentrated. The residue was applied onto a silica gel column and eluted with ethyl acetate/petroleum ether (1:1). The collected fractions were combined and concentrated. This resulted in 1.4 g (69.47%) of (2, 4-dibromopyridin-3-yl) methanol as a light yellow solid. LCMS-2 (ES, m/z): M+1: 266

Synthesis of 2,4-dibromo-3-[(oxan-2-yloxy) methyl] pyridine: Into a 100-mL round-bottom flask, was placed (2, 4-dibromopyridin-3-yl) methanol (1.40 g, 5.245 mmol, 1.00 equiv), DCM (30.00 mL, 0.353 mmol, 0.07 equiv), PPTS (131.81 mg, 0.525 mmol, 0.10 equiv), DHP (661.79 mg, 7.868 mmol, 1.50 equiv). The resulting solution was stirred for overnight at 45 degrees C. in an oil bath. The reaction was then quenched by the addition of 30 mL of water. The resulting solution was extracted with 3×30 mL of dichloromethane concentrated. The residue was applied onto a silica gel column and eluted with ethyl acetate/petroleum ether (1:1). The collected fractions were combined and concentrated. This resulted in 1.5 g of 2,4-dibromo-3-[(oxan-2-yloxy) methyl] pyridine as colorless oil. LCMS-3 (ES, m/z): M+1: 350

Synthesis of 5-bromo-3-[(4-fluoro-3-nitrophenyl)amino]-1-methylpyrazin-2-one: Into a 250-mL round-bottom flask, was placed 4-fluoro-3-nitroaniline (10.00 g, 64.055 mmol, 1.00 equiv), 3,5-dibromo-1-methylpyrazin-2-one (17.16 g, 64.052 mmol, 1.00 equiv), NMP (30 ml). The resulting solution was stirred for 1 h at 140 degrees C. in an oil bath. The resulting solution was diluted with 300 mL of EA. The solids were collected by filtration. This resulted in 13 g (59.15%) of 5-bromo-3-[(4-fluoro-3-nitrophenyl)amino]-1-methylpyrazin-2-one as a brown solid. LCMS-4 (ES, m/z): M+1: 343/345

Synthesis of tert-butyl (3S)-4-[4-[(6-bromo-4-methyl-3-oxopyrazin-2-yl)amino]-2-nitrophenyl]-3-methylpiperazine-1-carboxylate: Into a 50-mL round-bottom flask, was placed 5-bromo-3-[(4-fluoro-3-nitrophenyl)amino]-1-methylpyrazin-2-one (10 g, 29.2 mmol, 1.00 equiv), NMP (40.00 mL), tert-butyl (3S)-3-methylpiperazine-1-carboxylate (5.8 g, 5.842 mmol, 1.00 equiv), DIEA (2.26 g, 17.487 mmol, 3.00 equiv). The resulting solution was stirred for 40 h at 120° C. in an oil bath. The resulting solution was diluted with 100 mL of H₂O. The resulting solution was extracted with 3×50 mL of dichloromethane/methanol (10:1). The resulting mixture was washed with 3×20 ml of NaCl. The resulting mixture was concentrated. The residue was applied onto a silica gel column with dichloromethane/methanol (10:1). This resulted in 10 g (57%) of tert-butyl (3S)-4-[4-[(6-bromo-4-methyl-3-oxopyrazin-2-yl)amino]-2-nitrophenyl]-3-methylpiperazine-1-carboxylate as a brown solid. LCMS-5 (ES, m/z): M+1: 523

Synthesis of 5-bromo-1-methyl-3-([4-[(2S)-2-methylpiperazin-1-yl]-3-nitrophenyl]amino)pyrazin-2-one: Into a 100-mL round-bottom flask, was placed tert-butyl (3S)-4-[4-[(6-bromo-4-methyl-3-oxopyrazin-2-yl)amino]-2-nitrophenyl]-3-methylpiperazine-1-carboxylate (10.00 g, 1 equiv, 60%), HCl (2M) in 1,4-dioxane (100 mL). The resulting solution was stirred for 14 h at room temperature. The resulting mixture was concentrated. The resulting solution was diluted with 30 mL of H₂O. The pH value of the solution was adjusted to 8 with NH₃—H₂O. The resulting solution was extracted with 3×15 mL of dichloromethane concentrated. The residue was applied onto a silica gel column with dichloromethane/methanol (10:1). This resulted in 5 g of 5-bromo-1-methyl-3-([4-[(2S)-2-methylpiperazin-1-yl]-3-nitrophenyl]amino)pyrazin-2-one as a red solid. LCMS-6 (ES, m/z): M+1: 423

Synthesis of 5-bromo-1-methyl-3-([4-[(2S)-2-methyl-4-(oxetan-3-yl)piperazin-1-yl]-3-nitrophenyl]amino)pyrazin-2-one: Into a 250-mL round-bottom flask, was placed 5-bromo-1-methyl-3-([4-[(2S)-2-methylpiperazin-1-yl]-3-nitrophenyl]amino)pyrazin-2-one (4.00 g, 9.450 mmol, 1.00 equiv), 3-oxetanone (0.89 g, 12.350 mmol, 1.31 equiv), THF (40.00 mL), AcOH (0.80 mL). This was followed by the addition of NaBH(AcO)₃ (3.00 g, 14.155 mmol, 1.50 equiv) dropwise with stirring at room temperature. The resulting solution was stirred for 4 hr at room temperature. The reaction was then quenched by the addition of 10 mL of water. The resulting mixture was concentrated. The resulting solution was diluted with 40 mL of DCM. The resulting mixture was washed with 1×10 ml Na₂CO₃ (aq). The mixture was dried over anhydrous sodium sulfate and concentrated. The residue was applied onto a silica gel column with and eluted with dichloromethane/methanol (10:1). This resulted in 3 g (66.23%) of 5-bromo-1-methyl-3-([4-[(2S)-2-methyl-4-(oxetan-3-yl)piperazin-1-yl]-3-nitrophenyl]amino)pyrazin-2-one as a brown solid. LCMS-7 (ES, m/z): M+1: 479/481

Synthesis of 3-([3-amino-4-[(2S)-2-methyl-4-(oxetan-3-yl)piperazin-1-yl]phenyl]amino)-5-bromo-1-methylpyrazin-2-one: Into a 250-mL round-bottom flask, was placed 5-bromo-1-methyl-3-([4-[(2S)-2-methyl-4-(oxetan-3-yl)piperazin-1-yl]-3-nitrophenyl]amino)pyrazin-2-one (3.00 g, 6.259 mmol, 1.00 equiv), Fe (1.40 g, 25.035 mmol, 4.00 equiv), NH₄Cl (2.01 g, 37.576 mmol, 6.00 equiv), EtOH (30.00 mL), H₂O (30.00 mL). The resulting solution was stirred for 2 hr at 80 degrees C. in an oil bath. The solids were filtered out. The resulting mixture was concentrated. The resulting solution was diluted with 200 mL of DCM. The pH value of the solution was adjusted to 8 with NH3-H2O. The resulting mixture was washed with 1×20 ml of H₂O. The resulting mixture was washed with 1×20 mL of NaCl(aq). The mixture was dried over anhydrous sodium sulfate. The residue was applied onto a silica gel column and eluted with dichloromethane/methanol (10:1). This resulted in 2.5 g (88.89%) of 3-([3-amino-4-[(2S)-2-methyl-4-(oxetan-3-yl)piperazin-1-yl]phenyl]amino)-5-bromo-1-methylpyrazin-2-one as a brown solid. LCMS-8 (ES, m/z): M+1: 449/451

Synthesis of N-[5-[(6-bromo-4-methyl-3-oxopyrazin-2-yl)amino]-2-[(2S)-2-methyl-4-(oxetan-3-yl)piperazin-1-yl]phenyl]prop-2-enamide: Into a 100-mL round-bottom flask, was placed 3-([3-amino-4-[(2S)-2-methyl-4-(oxetan-3-yl)piperazin-1-yl]phenyl]amino)-5-bromo-1-methylpyrazin-2-one (2.50 g, 5.564 mmol, 1.00 equiv), DCM (30.00 mL, 471.901 mmol, 84.82 equiv), DIEA (1.44 g, 11.142 mmol, 2.00 equiv). This was followed by the addition of acryloyl chloride (0.65 g, 7.182 mmol, 1.29 equiv), in portions at 0 degrees C. The resulting solution was stirred for 1 hr at 0 degrees C. in a water/ice bath. The reaction was then quenched by the addition of 1 mL of MeOH. The resulting mixture was concentrated. The residue was applied onto a silica gel column and eluted with dichloromethane/methanol (10:1). This resulted in 2.8 g (80.98%) of N-[5-[(6-bromo-4-methyl-3-oxopyrazin-2-yl)amino]-2-[(2S)-2-methyl-4-(oxetan-3-yl)piperazin-1-yl]phenyl]prop-2-enamide as a yellow solid. LCMS-9 (ES, m/z): M+1: 503/505

Synthesis of N-(methoxymethyl)-N-methyl-4,5,6,7-tetrahydro-1-benzothiophene-2-carboxamide: Into a 250-mL 3-necked round-bottom flask purged and maintained with an inert atmosphere of nitrogen, was placed 4,5,6,7-tetrahydro-1-benzothiophene-2-carboxylic acid (8.0 g, 43.95 mmol, 1.0 equiv), DMF (193 mg, 2.197 mmol, 0.05 equiv), DCM(150 ml).

This was followed by the addition of oxalyl chloride (6.1 g, 48.35 mmol, 1.1 equiv) dropwise with stirring at 0 degrees C. The resulting solution was stirred for 1 h in a water/ice bath. To this was added TEA (13.3 g, 131.85 mmol, 3.0 equiv) and N,O-dimethylhydroxylamine HCl salt (4.3 g, 43.95 mmol, 1.0 equiv) at 0 degrees C. The resulting solution was stirred for 2 h at room temperature. The resulting solution was diluted with 100 mL of water. The resulting solution was extracted with 3×150 mL of dichloromethane and the organic layers combined. The resulting mixture was washed with 2×100 ml of water and 1×100 mL of brine. The mixture was dried over anhydrous sodium sulfate and concentrated. The residue was applied onto a silica gel column with ethyl acetate/petroleum ether (1:10). This resulted in 9.0 g of N-(methoxymethyl)-N-methyl-4,5,6,7-tetrahydro-1-benzothiophene-2-carboxamide as a white solid. LCMS-10 (ES, m/z): M+1: 226

Synthesis of 3-chloro-1-(4,5,6,7-tetrahydro-1-benzothiophen-2-yl)propan-1-one: Into a 250-mL 3-necked round-bottom flask purged and maintained with an inert atmosphere of nitrogen, was placed N-methoxy-N-methyl-4,5,6,7-tetrahydro-1-benzothiophene-2-carboxamide (8.00 g, 35.560 mmol, 1.00 equiv), THF (40.00 mL). This was followed by the addition of bromo(ethenyl)magnesium (1M in THF) (160.00 mL, 142.220 mmol, 4.00 equiv) dropwise with stirring at −10 degrees C. The resulting solution was stirred for 4 h at 0 degrees C. in an ice/salt bath. The reaction was then quenched by the addition of 40 mL of 2M HCl. The resulting solution was extracted with 2×100 mL of ethyl acetate and the organic layers combined. The resulting mixture was washed with 2×100 ml of water and 1×100 mL of brine. The mixture was dried over anhydrous sodium sulfate and concentrated. The resulting solution was diluted with 80 mL of DCM. The residue was dissolved in 40 mL of 2M in Et₂O. The resulting mixture was concentrated. The residue was applied onto a silica gel column with ethyl acetate/petroleum ether (1:5). This resulted in 2.3 g of 3-chloro-1-(4,5,6,7-tetrahydro-1-benzothiophen-2-yl)propan-1-one as yellow oil. LCMS-11 (ES, m/z): M+1: 229

Synthesis of 7-thiatricyclo [6.4.0.0\hat{ }[2, 6]] dodeca-1(8), 2(6)-dien-5-one: Into a 100-mL round-bottom flask, was placed 3-chloro-1-(4,5,6,7-tetrahydro-1-benzothiophen-2-yl)propan-1-one (2.30 g, 10.090 mmol, 1.00 equiv), H₂SO₄ (20.00 mL). The resulting solution was stirred for 16 hr at 95 degrees C. in an oil bath. The reaction mixture was cooled to room temperature with a water/ice bath. The resulting solution was diluted with 50 mL of water. The resulting solution was extracted with 2×50 mL of ethyl acetate and the organic layers combined. The resulting mixture was washed with 1×50 ml of brine. The mixture was dried over anhydrous sodium sulfate and concentrated. The residue was applied onto a silica gel column with ethyl acetate/petroleum ether (1:5). This resulted in 0.8 g of 7-thiatricyclo[6.4.0.0\hat{ }[2,6]]dodeca-1(8),2(6)-dien-5-one as brown oil. LCMS-12 (ES, m/z): M+1: 193

Synthesis of N-[(5E)-7-thiatricyclo[6.4.0.0\hat{ }[2,6]]dodeca-1(8),2(6)-dien-5-ylidene]hydroxylamine: Into a 100-mL 3-necked round-bottom flask purged and maintained with an inert atmosphere of nitrogen, was placed NH₂OH·HCl (1.41 g, 20.313 mmol, 5.00 equiv), MeOH (30.00 mL). This was followed by the addition of NaOAc (1.66 g, 20.313 mmol, 5.00 equiv) at 0 degrees C. and the solution was stirred for 30 min. To this was added 7-thiatricyclo[6.4.0.0\hat{ }[2,6]]dodeca-1(8),2(6)-dien-5-one (780.00 mg, 4.063 mmol, 1.00 equiv) at 0 degrees C. The resulting solution was stirred for 16 h at room temperature. The resulting mixture was concentrated. This resulted in 300 mg of N-[(5E)-7-thiatricyclo[6.4.0.0\hat{ }[2,6]]dodeca-1(8),2(6)-dien-5-ylidene]hydroxylamine as brown oil.

LCMS-13 (ES, m/z): M+1: 208

Synthesis of 8-thia-5-azatricyclo[7.4.0.0\hat{ }[2,7]]trideca-1(9),2(7)-dien-6-one: Into a 50-mL round-bottom flask purged and maintained with an inert atmosphere of nitrogen, was placed N-[(5E)-7-thiatricyclo[6.4.0.0\hat{ }[2,6]]dodeca-1(8),2(6)-dien-5-ylidene]hydroxylamine (295.00 mg, 1.425 mmol, 1.00 equiv), PPA (6.00 mL). The resulting solution was stirred for 18 h at 80 degrees C. in an oil bath. The reaction mixture was cooled to room temperature with a water bath. The resulting solution was diluted with 20 mL of water. The solids were collected by filtration. This resulted in 260 mg of 8-thia-5-azatricyclo[7.4.0.0\hat{ }[2,7]]trideca-1(9),2(7)-dien-6-one as an off-white solid. LCMS-14 (ES, m/z): M+1: 208

Synthesis of 5-[4-bromo-3-[(oxan-2-yloxy) methyl] pyridin-2-yl]-8-thia-5-azatricyclo [7.4.0.0\hat{ }[2, 7]] trideca-1(9), 2 (7)-dien-6-one: Into a 50-mL round-bottom flask purged and maintained with an inert atmosphere of nitrogen, was placed 8-thia-5-azatricyclo[7.4.0.0\hat{ }[2,7]]trideca-1(9),2(7)-dien-6-one (260.00 mg, 1.251 mmol, 1.00 equiv), 2,4-dibromo-3-[(oxan-2-yloxy)methyl]pyridine (873.00 mg, 1.875 mmol, 1.50 equiv), CuI (182.00 mg, 0.751 mmol, 0.60 equiv), Cs₂CO₃ (1.01 g, 2.502 mmol, 2.00 equiv), DMA (10.00 mL), 1,10-phenanthroline (182.00 mg, 0.751 mmol, 0.60 equiv). The resulting solution was stirred for 4 h at 110 degrees C. in an oil bath. The reaction mixture was cooled to room temperature with a water bath. The solids were filtered out. The resulting solution was diluted with 20 mL of water. The resulting solution was extracted with 2×20 mL of ethyl acetate and the organic layers combined. The resulting mixture was washed with 3×20 ml of water. The resulting mixture was washed with 1×20 mL of brine. The mixture was dried over anhydrous sodium sulfate and concentrated. The residue was applied onto a silica gel column with dichloromethane/methanol (10:1). This resulted in 360 mg of 5-[4-bromo-3-[(oxan-2-yloxy)methyl]pyridin-2-yl]-8-thia-5-azatricyclo[7.4.0.0\hat{ }[2,7]]trideca-1(9),2(7)-dien-6-one as dark brown oil. LCMS-15 (ES, m/z): M+1: 477

Synthesis of 3-[(oxan-2-yloxy)methyl]-2-[6-oxo-8-thia-5-azatricyclo[7.4.0.0\hat{ }[2,7]]trideca-1(9),2(7)-dien-5-yl]pyridin-4-ylboronic acid: Into a 50-mL round-bottom flask purged and maintained with an inert atmosphere of nitrogen, was placed 5-[4-bromo-3-[(oxan-2-yloxy)methyl]pyridin-2-yl]-8-thia-5-azatricyclo[7.4.0.0\hat{ }[2,7]]trideca-1(9),2(7)-dien-6-one (360.00 mg, 0.756 mmol, 1.00 equiv), bis(pinacolato)diboron (102.00 mg, 1.891 mmol, 2.50 equiv), KOAc (222.00 mg, 2.268 mmol, 3.00 equiv), Pd(dppf)Cl₂ (56.00 mg, 0.076 mmol, 0.10 equiv), Dioxane (20.00 mL). The resulting solution was stirred for 2 h at 100 degrees C. in an oil bath. The reaction mixture was cooled to room temperature with a water bath. The solids were filtered out. The resulting mixture was concentrated. The crude product was purified by Flash-Prep-HPLC with the following conditions (CombiFlash-1): Column, C18 silica gel; mobile phase, H₂O: ACN=20% increasing to H₂O: ACN=65% within 10 min; Detector, 220 nm. This resulted in 180 mg of 3-[(oxan-2-yloxy)methyl]-2-[6-oxo-8-thia-5-azatricyclo[7.4.0.0\hat{ }[2,7]]trideca-1(9),2(7)-dien-5-yl]pyridin-4-ylboronic acid as an off-white solid. LCMS-16 (ES, m/z): M+1: 443

Synthesis of 5-[1-hydroxy-3H-[1, 2] oxaborolo [4, 3-c] pyridin-4-yl]-8-thia-5-azatricyclo [7.4.0.0\hat{ }[2, 7]] trideca-1(9), 2(7)-dien-6-one: Into a 50-mL round-bottom flask, was placed 3-[(oxan-2-yloxy)methyl]-2-[6-oxo-8-thia-5-azatricyclo[7.4.0.0\hat{ }[2,7]]trideca-1(9),2(7)-dien-5-yl]pyridin-4-ylboronic acid (160.00 mg, 0.362 mmol, 1.00 equiv), 4N HCl in Dioxane (5.00 mL). The resulting solution was stirred for 1 hr at room temperature. The solids were collected by filtration. The solids was washed by water 10 ml. This resulted in 100 mg of 5-[1-hydroxy-3H-[1,2]oxaborolo[4,3-c]pyridin-4-yl]-8-thia-5-azatricyclo[7.4.0.0\hat{ }[2,7]]trideca-1(9),2(7)-dien-6-one as an off-white solid. LCMS-17 (ES, m/z): M+1: 341

Synthesis of N-[5-([6-[3-(hydroxymethyl)-2-[6-oxo-8-thia-5-azatricyclo[7.4.0.0\hat{ }[2,7]]trideca-1(9),2(7)-dien-5-yl]pyridin-4-yl]-4-methyl-3-oxopyrazin-2-yl]amino)-2-[(2S)-2-methyl-4-(oxetan-3-yl)piperazin-1-yl]phenyl]prop-2-enamide: Into a 50-mL round-bottom flask purged and maintained with an inert atmosphere of nitrogen, was placed 5-[1-hydroxy-3H-[1,2]oxaborolo[4,3-c]pyridin-4-yl]-8-thia-5-azatricyclo[7.4.0.0\hat{ }[2,7]]trideca-1(9),2(7)-dien-6-one (80.00 mg, 0.235 mmol, 1.00 equiv), N-[5-[(6-bromo-4-methyl-3-oxopyrazin-2-yl)amino]-2-[(2S)-2-methyl-4-(oxetan-3-yl)piperazin-1-yl]phenyl]prop-2-enamide (100.00 mg, 0.235 mmol, 1.00 equiv), K₃PO₄ (100.00 mg, 0.471 mmol, 2.00 equiv), Toluene (5.00 mL), BrettPhos Pd G3 (21.00 mg, 0.024 mmol, 0.10 equiv). The resulting solution was stirred for 1 hr at 90 degrees C. in an oil bath. The reaction mixture was cooled to room temperature with a water bath. The solids were filtered out. The resulting mixture was concentrated. The residue was applied onto a silica gel column with dichloromethane/methanol (10:1). The crude product (100 mg) was purified by Prep-HPLC with the following conditions: column, X-Bridge Prep C18 19*150 mm 5 um; mobile phase, A: water (it contains 10 mM NH₄HCO₃ 0.05% ammonia); B: ACN; Gradient: 20-45% B in 8 min; Flow rate: 20 mL/min; detector, UV 220 nm. The collected solution was concentrated under vacuum to remove CH₃CN and the resulting solution was dried by lyophilization. This resulted in 12 mg of N-[5-([6-[3-(hydroxymethyl)-2-[6-oxo-8-thia-5-azatricyclo[7.4.0.0\hat{ }[2,7]]trideca-1(9),2(7)-dien-5-yl]pyridin-4-yl]-4-methyl-3-oxopyrazin-2-yl]amino)-2-[(2S)-2-methyl-4-(oxetan-3-yl)piperazin-1-yl]phenyl]prop-2-enamide as a white solid. LCMS-18 (ES, m/z): M+1: 737, ¹H NMR (300 MHz, DMSO-d₆, ppm) δ 9.26 (s, 1H), 9.19 (s, 1H), 9.10 (s, 1H), 8.47-8.49 (d, J=6.0 Hz, 1H), 7.91-7.93 (d, J=6.0 Hz, 1H), 7.72 (s, 1H), 7.59-7.61 (d, J=6.0 Hz, 1H), 7.25-7.27 (d, J=6.0 Hz, 1H), 6.57-6.60 (m, 1H), 6.29-6.31 (d, J=6.0 Hz, 1H), 5.78-5.81 (d, J=9.0 Hz, 1H), 4.95 (m, 1H), 4.53 (m, 6H), 4.18 (m, 1H), 3.86 (m, 1H), 3.57 (s, 3H), 3.46-3.48 (m, 1H), 3.10 (s, 1H), 3.01-2.87 (m, 2H), 2.78-2.70 (m, 6H), 2.68-2.73 (m, 2H), 2.21-2.30 (m, 1H), 1.92 (t, J=10.0 Hz, 1H), 1.81 (m, 4H), 0.72-0.74 (d, J=6.0 Hz, 3H).

### Compound 5: Preparation of N-[5-([6-[3-(hydroxymethyl)-2-[6-oxo-8-thia-5-azatricyclo[7.4.0.0\hat{ }[2,7]]trideca-1(9), 2(7)-dien-5-yl]pyridin-4-yl]-4-methyl-3-oxopyrazin-2-yl]amino)-2-[(2S)-2-methyl-4-(oxan-4-yl) piperazin-1-yl]phenyl]prop-2-enamide

Synthesis of 2, 4-dibromopyridine-3-carbaldehyde Into a 1000-mL 3-necked round-bottom flask, was placed 2, 4-dibromopyridine (40.00 g, 168.852 mmol, 1.00 equiv), THF (400.00 mL). This was followed by the addition of LDA (2M in hexane, 126.60 mL, 1.50 equiv) dropwise with stirring at −78 degrees C. The resulting solution was stirred for 1 h at −78 degrees C. Then DMF (16.04 ml, 219.507 mmol, 1.30 equiv) dropwise with stirring at −78 degrees C. The resulting solution was stirred for 0.5 h at −78 degrees C. The reaction was then quenched by the addition of 500 mL of NH₄Cl. The resulting solution was extracted with 3×500 mL of ethyl acetate concentrated. The residue was applied onto a silica gel column and eluted with ethyl acetate/petroleum ether (0:1-1:1). This resulted in 24.4 g (54.55%) of 2, 4-dibromopyridine-3-carbaldehyde as a white solid. LC-MS-1 (ES, m/z): M+1: 264

Synthesis of (2, 4-dibromopyridin-3-yl) methanol Into a 100-mL round-bottom flask, was placed 2, 4-dibromopyridine-3-carbaldehyde (2.00 g, 7.550 mmol, 1.00 equiv), EtOH (30.00 mL). This was followed by the addition of NaBH₄ (285.64 mg, 7.550 mmol, 1 equiv), in portions at 0 degrees C. The resulting solution was stirred for 3 h at room temperature. The reaction was then quenched by the addition of 30 mL of water. The resulting solution was extracted with 3×30 mL of ethyl acetate concentrated. The residue was applied onto a silica gel column and eluted with ethyl acetate/petroleum ether (1:1). The collected fractions were combined and concentrated. This resulted in 1.4 g (69.47%) of (2, 4-dibromopyridin-3-yl) methanol as a light yellow solid. LC-MS-2 (ES, m/z): M+1: 266

Synthesis of 2,4-dibromo-3-[(oxan-2-yloxy) methyl] pyridine Into a 100-mL round-bottom flask, was placed (2, 4-dibromopyridin-3-yl) methanol (1.40 g, 5.245 mmol, 1.00 equiv), DCM (30.00 mL, 0.353 mmol, 0.07 equiv), PPTS (131.81 mg, 0.525 mmol, 0.10 equiv), DHP (661.79 mg, 7.868 mmol, 1.50 equiv). The resulting solution was stirred for overnight at 45 degrees C. in an oil bath. The reaction was then quenched by the addition of 30 mL of water. The resulting solution was extracted with 3×30 mL of dichloromethane concentrated. The residue was applied onto a silica gel column and eluted with ethyl acetate/petroleum ether (1:1). The collected fractions were combined and concentrated. This resulted in 1.5 g of 2,4-dibromo-3-[(oxan-2-yloxy) methyl] pyridine as colorless oil. LC-MS-3 (ES, m/z): M+1: 350

Synthesis of 5-bromo-3-[(4-fluoro-3-nitrophenyl) amino]-1-methylpyrazin-2-one Into a 10 L round-bottom flask, was placed 4-fluoro-3-nitroaniline (586.47 g, 3.759 mol, 1.00 equiv), 3, 5-dibromo-1-methylpyrazin-2-one (1000 g, 3.759 mol, 1.00 equiv), NMP (3000 ml). The resulting solution was stirred for 1 h at 135-140 degrees C. in an oil bath. The resulting solution was cooled and diluted with 3 L of H₂O. The solids were collected by filtration. The solids washed by EA (2×1 L). This resulted in 980 g (90% purity) of 5-bromo-3-[(4-fluoro-3-nitrophenyl) amino]-1-methylpyrazin-2-one as a brown solid. LC-MS-4 (ES, m/z): M+1: 343/345

Synthesis of tert-butyl (3S)-4-[4-[(6-bromo-4-methyl-3-oxopyrazin-2-yl) amino]-2-nitrophenyl]-3-methylpiperazine-1-carboxylate Into a 10 L round-bottom flask, was placed 5-bromo-3-[(4-fluoro-3-nitrophenyl) amino]-1-methylpyrazin-2-one (1000 g, 2923.9 mmol, 1.00 equiv), NMP (4000.00 mL), tert-butyl (3S)-3-methylpiperazine-1-carboxylate (701.7 g, 3508.7 mmol, 1.20 equiv), DIEA (1131.55 g, 8771.7 mmol, 3.00 equiv). The resulting solution was stirred for 48 h at 120° C. in an oil bath. The resulting solution was diluted with 10000 mL of H₂O. The solids were collected by filtration. This resulted in 1200 g of tert-butyl (3S)-4-[4-[(6-bromo-4-methyl-3-oxopyrazin-2-yl) amino]-2-nitrophenyl]-3-methylpiperazine-1-carboxylate as a brown crude solid. LC-MS-5 (ES, m/z): M+1: 523/525

Synthesis of 5-bromo-1-methyl-3-([4-[(2S)-2-methylpiperazin-1-yl]-3-nitrophenyl] amino) pyrazin-2-one Into a 10 L round-bottom flask, was placed tert-butyl (3S)-4-[4-[(6-bromo-4-methyl-3-oxopyrazin-2-yl) amino]-2-nitrophenyl]-3-methylpiperazine-1-carboxylate (1200 g, 1 equiv), dioxane (3000 ml), HCl (4M) in 1,4-dioxane (3000.00 mL). The resulting solution was stirred for 13 h at room temperature. The solids were collected by filtration. Filter cake was washed by EA. The filter cake was diluted with 300 mL of H₂O. The pH value of the solution was adjusted to 8 with NaHCO₃. The solids were collected by filtration. This resulted in 900 g (87.8% purity) of 5-bromo-1-methyl-3-([4-[(2S)-2-methylpiperazin-1-yl]-3-nitrophenyl] amino) pyrazin-2-one as a red solid. LC-MS-6 (ES, m/z): M+1: 423/425

Synthesis of 5-bromo-1-methyl-3-([4-[(2S)-2-methyl-4-(oxan-4-yl)piperazin-1-yl]-3-nitrophenyl] amino)pyrazin-2-one Into a 50-mL round-bottom flask, was placed 5-bromo-1-methyl-3-([4-[(2S)-2-methylpiperazin-1-yl]-3-nitrophenyl]amino)pyrazin-2-one (250 mg, 0.591 mmol, 1.00 equiv), 4H-pyran-4-one, tetrahydro-(70.96 mg, 0.709 mmol, 1.20 equiv), THF (5 ml), AcOH (5 drops), NaBH(AcO)₃ (250.36 mg, 1.181 mmol, 2.00 equiv). The resulting solution was stirred for 14 h at 30° C. in an oil bath. The resulting mixture was concentrated. The residue was applied onto a silica gel column with dichloromethane/methanol (10:1). This resulted in 220 mg (73.41%) of 5-bromo-1-methyl-3-([4-[(2S)-2-methyl-4-(oxan-4-yl)piperazin-1-yl]-3-nitrophenyl]amino)pyrazin-2-one as a white solid. LC-MS-7 (ES, m/z): M+1: 507/509

Synthesis of 3-([3-amino-4-[(2S)-2-methyl-4-(oxan-4-yl)piperazin-1-yl]phenyl]amino)-5-bromo-1-methylpyrazin-2-one Into a 250-mL round-bottom flask, was placed 5-bromo-1-methyl-3-([4-[(2S)-2-methyl-4-(oxan-4-yl)piperazin-1-yl]-3-nitrophenyl]amino)pyrazin-2-one (10.00 g, 19.709 mmol, 1.00 equiv), EtOH (90.00 mL), H₂O (30.00 mL), Fe (4.40 g, 0.079 mmol, 4.00 equiv), NH₄Cl (8.43 g, 0.158 mmol, 8.00 equiv). The resulting solution was stirred for 1.5 h at 80 degrees C. in an oil bath. The solids were filtered out. The resulting mixture was concentrated. This resulted in 7.7 g (81.84%) of 3-([3-amino-4-[(2S)-2-methyl-4-(oxan-4-yl)piperazin-1-yl] phenyl] amino)-5-bromo-1-methylpyrazin-2-one as a yellow solid. LC-MS-8 (ES, m/z): M+1: 477/479

Synthesis of N-[5-[(6-bromo-4-methyl-3-oxopyrazin-2-yl)amino]-2-[(2S)-2-methyl-4-(oxan-4-yl) piperazin-1-yl]phenyl]prop-2-enamide Into a 250-mL round-bottom flask, was placed 3-([3-amino-4-[(2S)-2-methyl-4-(oxan-4-yl)piperazin-1-yl]phenyl]amino)-5-bromo-1-methylpyrazin-2-one (3.50 g, 7.331 mmol, 1.00 equiv), DCM (125.00 mL), DIEA (1.90 g, 0.015 mmol, 2.00 equiv). This was followed by the addition of acryloyl chloride (663.55 mg, 7.331 mmol, 1.00 equiv) dropwise with stirring at 0 degrees C. The resulting solution was stirred for 2 h at room temperature. The resulting mixture was concentrated. The residue was applied onto a silica gel column with dichloromethane/methanol (10:1). The collected fractions were combined and concentrated. This resulted in 4.3 g (110.36%) of N-[5-[(6-bromo-4-methyl-3-oxopyrazin-2-yl)amino]-2-[(2S)-2-methyl-4-(oxan-4-yl)piperazin-1-yl]phenyl]prop-2-enamide as a yellow solid. LC-MS-9 (ES, m/z): M+1: 531/533

Synthesis of N-(methoxymethyl)-N-methyl-4, 5, 6, 7-tetrahydro-1-benzothiophene-2-carboxamide Into a 250-mL 3-necked round-bottom flask purged and maintained with an inert atmosphere of nitrogen, was placed 4,5,6,7-tetrahydro-1-benzothiophene-2-carboxylic acid (8.0 g, 43.95 mmol, 1.0 equiv), DMF (193 mg, 2.197 mmol, 0.05 equiv), DCM (150 ml). This was followed by the addition of oxalyl chloride (6.1 g, 48.35 mmol, 1.1 equiv) dropwise with stirring at 0 degrees C. The resulting solution was stirred for 1 h in a water/ice bath. To this was added TEA (13.3 g, 131.85 mmol, 3.0 equiv) and N,O-dimethylhydroxylamine HCl salt (4.3 g, 43.95 mmol, 1.0 equiv) at 0 degrees C. The resulting solution was stirred for 2 h at room temperature. The resulting solution was diluted with 100 mL of water. The resulting solution was extracted with 3×150 mL of dichloromethane and the organic layers combined. The resulting mixture was washed with 2×100 ml of water and 1×100 mL of brine. The mixture was dried over anhydrous sodium sulfate and concentrated. The residue was applied onto a silica gel column with ethyl acetate/petroleum ether (1:10). This resulted in 9.0 g of N-(methoxymethyl)-N-methyl-4, 5, 6, 7-tetrahydro-1-benzothiophene-2-carboxamide as a white solid. LC-MS-10 (ES, m/z): M+1: 226

Synthesis of 3-chloro-1-(4, 5, 6, 7-tetrahydro-1-benzothiophen-2-yl) propan-1-one Into a 250-mL 3-necked round-bottom flask purged and maintained with an inert atmosphere of nitrogen, was placed N-methoxy-N-methyl-4,5,6,7-tetrahydro-1-benzothiophene-2-carboxamide (8.00 g, 35.560 mmol, 1.00 equiv), THF (40.00 mL). This was followed by the addition of bromo(ethenyl)magnesium (1M in THF) (160.00 mL, 142.220 mmol, 4.00 equiv) dropwise with stirring at −10 degrees C. The resulting solution was stirred for 3 h at 0 degrees C. in an ice/salt bath. The reaction was then quenched by the addition of 40 mL of 2M HCl (aq). The resulting solution was extracted with 2×100 mL of ethyl acetate and the organic layers combined. The resulting mixture was washed with 2×100 ml of water and 1×100 mL of brine. The mixture was dried over anhydrous sodium sulfate and concentrated. The resulting solution was diluted with 80 mL of DCM. The residue was dissolved in 40 mL of 2M HCl(gas) in Et₂O. The resulting mixture was stirred for 3 h at R.T. Then the solution was concentrated. The residue was applied onto a silica gel column with ethyl acetate/petroleum ether (1:5). This resulted in 2.3 g of 3-chloro-1-(4, 5, 6, 7-tetrahydro-1-benzothiophen-2-yl) propan-1-one as yellow oil. LC-MS-11 (ES, m/z): M+1: 229

Synthesis of 1, 2, 5, 6, 7, 8-hexahydro-3H-benzo[b]cyclopenta[d]thiophen-3-one Into a 100-mL round-bottom flask, was placed 3-chloro-1-(4, 5, 6, 7-tetrahydro-1-benzothiophen-2-yl) propan-1-one (2.30 g, 10.090 mmol, 1.00 equiv), H₂SO₄ (20.00 mL). The resulting solution was stirred for 16 h at 95 degrees C. in an oil bath. The reaction mixture was cooled to room temperature with a water/ice bath. The resulting solution was diluted with 50 mL of water. The resulting solution was extracted with 2×50 mL of ethyl acetate and the organic layers combined. The resulting mixture was washed with 1×50 ml of brine. The mixture was dried over anhydrous sodium sulfate and concentrated. The residue was applied onto a silica gel column with ethyl acetate/petroleum ether (1:5). This resulted in 0.8 g of 1, 2, 5, 6, 7, 8-hexahydro-3H-benzo[b]cyclopenta[d]thiophen-3-one as brown oil. LC-MS-12 (ES, m/z): M+1: 193

Synthesis of (Z)-1, 2, 5, 6, 7, 8-hexahydro-3H-benzo[b]cyclopenta[d]thiophen-3-one oxime Into a 100-mL 3-necked round-bottom flask purged and maintained with an inert atmosphere of nitrogen, was placed NH₂OH·HCl (1.41 g, 20.313 mmol, 5.00 equiv), MeOH (30.00 mL). This was followed by the addition of NaOAc (1.66 g, 20.313 mmol, 5.00 equiv) at 0 degrees C. and the solution was stirred for 30 min at 0 degrees C. To this was added 1, 2, 5, 6, 7, 8-hexahydro-3H-benzo [b]cyclopenta[d]thiophen-3-one (780.00 mg, 4.063 mmol, 1.00 equiv) at 0 degrees C. The resulting solution was stirred for 18 h at room temperature. The resulting mixture was concentrated. The mixture was diluted with DCM (60 ml), then washed with water (2×30 ml) and brine (1×50 ml). The organic layers was combined and dried over anhydrous sodium sulfate and concentrated. The residue was applied onto a silica gel column with ethyl acetate/petroleum ether (1:1). This resulted in 300 mg of (Z)-1, 2, 5, 6, 7, 8-hexahydro-3H-benzo[b]cyclopenta[d]thiophen-3-one oxime as brown oil. LCMS-19: M+1: 208.

Synthesis of 3, 4, 5, 6, 7, 8-hexahydrobenzo [4, 5] thieno [2, 3-c] pyridin-1(2H)-one Into a 50-mL round-bottom flask purged and maintained with an inert atmosphere of nitrogen, was placed (Z)-1, 2, 5, 6, 7, 8-hexahydro-3H-benzo [b]cyclopenta[d]thiophen-3-one oxime (295.00 mg, 1.425 mmol, 1.00 equiv), PPA (6.00 mL). The resulting solution was stirred for 18 h at 80 degrees C. in an oil bath. The reaction mixture was cooled to room temperature with a water bath. The resulting solution was diluted with 20 mL of water. The mixture was extracted with 2×50 mL of ethyl acetate and the organic layers combined. The resulting mixture was washed with 1×50 ml of brine. The mixture was dried over anhydrous sodium sulfate and concentrated. The residue was applied onto a silica gel column with dichloromethane/methanol (5:1). This resulted in 260 mg of 3, 4, 5, 6, 7, 8-hexahydrobenzo [4, 5]thieno [2, 3-c] pyridin-1(2H)-one as an off-white solid. LCMS-20: M+1: 208. ¹H-NMR (300 MHz, DMSO-d₆, ppm) δ 3.37-3.43 (m, 2H), 2.73-2.76 (m, 2H), 2.61-2.65 (m, 2H), 2.44-2.48 (m, 2H), 1.74-1.80 (m, 4H).

Synthesis of 5-[4-bromo-3-[(oxan-2-yloxy) methyl] pyridin-2-yl]-8-thia-5-azatricyclo [7.4.0.0\hat{ }[2, 7]] trideca-1(9), 2 (7)-dien-6-one Into a 50-mL round-bottom flask purged and maintained with an inert atmosphere of nitrogen, was placed 8-thia-5-azatricyclo[7.4.0.0\hat{ }[2,7]]trideca-1(9),2(7)-dien-6-one (260.00 mg, 1.251 mmol, 1.00 equiv), 2,4-dibromo-3-[(oxan-2-yloxy)methyl]pyridine (873.00 mg, 1.875 mmol, 1.50 equiv), CuI (182.00 mg, 0.751 mmol, 0.60 equiv), Cs₂CO₃ (1.01 g, 2.502 mmol, 2.00 equiv), DMA (10.00 mL), 1,10-phenanthroline (182.00 mg, 0.751 mmol, 0.60 equiv). The resulting solution was stirred for 4 h at 110 degrees C. in an oil bath. The reaction mixture was cooled to room temperature with a water bath. The solids were filtered out. The resulting solution was diluted with 20 mL of water. The resulting solution was extracted with 2×20 mL of ethyl acetate and the organic layers combined. The resulting mixture was washed with 3×20 ml of water. The resulting mixture was washed with 1×20 mL of brine. The mixture was dried over anhydrous sodium sulfate and concentrated. The residue was applied onto a silica gel column with dichloromethane/methanol (10:1). This resulted in 360 mg of 5-[4-bromo-3-[(oxan-2-yloxy)methyl] pyridin-2-yl]-8-thia-5-azatricyclo[7.4.0.0\hat{ }[2,7]]trideca-1(9),2(7)-dien-6-one as dark brown oil. LC-MS-15 (ES, m/z): M+1: 477

Synthesis of 3-[(oxan-2-yloxy)methyl]-2-[6-oxo-8-thia-5-azatricyclo[7.4.0.0\hat{ }[2,7]]trideca-1(9), 2(7)-dien-5-yl]pyridin-4-ylboronic acid Into a 50-mL round-bottom flask purged and maintained with an inert atmosphere of nitrogen, was placed 5-[4-bromo-3-[(oxan-2-yloxy)methyl]pyridin-2-yl]-8-thia-5-azatricyclo[7.4.0.0\hat{ }[2,7]]trideca-1(9), 2(7)-dien-6-one (360.00 mg, 0.756 mmol, 1.00 equiv), bis(pinacolato)diboron (102.00 mg, 1.891 mmol, 2.50 equiv), KOAc (222.00 mg, 2.268 mmol, 3.00 equiv), Pd(dppf)Cl₂ (56.00 mg, 0.076 mmol, 0.10 equiv), Dioxane (20.00 mL). The resulting solution was stirred for 2 h at 100 degrees C. in an oil bath. The reaction mixture was cooled to room temperature with a water bath. The solids were filtered out. The resulting mixture was concentrated. The crude product was purified by Flash-Prep-HPLC with the following conditions (CombiFlash-1): Column, C18 silica gel; mobile phase, H₂O:ACN=20% increasing to H₂O:ACN=65% within 10 min; Detector, 220 nm. This resulted in 180 mg of 3-[(oxan-2-yloxy)methyl]-2-[6-oxo-8-thia-5-azatricyclo[7.4.0.0\hat{ }[2,7]]trideca-1(9),2(7)-dien-5-yl]pyridin-4-ylboronic acid as an off-white solid. LC-MS-16 (ES, m/z): M+1: 443

Synthesis of 5-[1-hydroxy-3H-[1, 2] oxaborolo [4, 3-c] pyridin-4-yl]-8-thia-5-azatricyclo [7.4.0.0\hat{ }[2, 7]] trideca-1(9), 2(7)-dien-6-one Into a 50-mL round-bottom flask, was placed 3-[(oxan-2-yloxy)methyl]-2-[6-oxo-8-thia-5-azatricyclo [7.4.0.0\hat{ }[2,7]]trideca-1(9),2(7)-dien-5-yl]pyridin-4-ylboronic acid (160.00 mg, 0.362 mmol, 1.00 equiv), 4N HCl in Dioxane (5.00 mL). The resulting solution was stirred for 1 h at room temperature. The solids were collected by filtration. The solids was washed by water 10 ml. This resulted in 100 mg of 5-[1-hydroxy-3H-[1,2]oxaborolo[4,3-c]pyridin-4-yl]-8-thia-5-azatricyclo[7.4.0.0\hat{ }[2,7]]trideca-1(9),2(7)-dien-6-one as an off-white solid. LC-MS-17 (ES, m/z): M+1: 341

Synthesis of N-[5-([6-[3-(hydroxymethyl)-2-[6-oxo-8-thia-5-azatricyclo[7.4.0.0\hat{ }[2,7]]trideca-1(9), 2(7)-dien-5-yl]pyridin-4-yl]-4-methyl-3-oxopyrazin-2-yl]amino)-2-[(2S)-2-methyl-4-(oxan-4-yl) piperazin-1-yl]phenyl]prop-2-enamide Into a 50-mL round-bottom flask purged and maintained with an inert atmosphere of nitrogen, was placed N-[5-[(6-bromo-4-methyl-3-oxopyrazin-2-yl)amino]-2-[(2S)-2-methyl-4-(oxan-4-yl)piperazin-1-yl] phenyl]prop-2-enamide (75.00 mg, 0.142 mmol, 1.00 equiv), 5-[1-hydroxy-3H-[1,2]oxaborolo[4,3-c] pyridin-4-yl]-8-thia-5-azatricyclo[7.4.0.0\hat{ }[2,7]]trideca-1(9),2(7)-dien-6-one (90.00 mg, 0.283 mmol, 2.00 equiv), K₃PO₄ (130.00 mg, 0.425 mmol, 3.00 equiv), Toluene (8.00 mL), H₂O (0.80 mL), BrettPhos Pd G3 (8.00 mg, 0.028 mmol, 0.20 equiv). The resulting solution was stirred for 2 h at 90 degrees C. in an oil bath. The reaction mixture was cooled to room temperature with a water bath. The resulting solution was diluted with 50 mL of EtOAc. The solids were filtered out. The resulting mixture was washed with 2×20 ml of water and 1×20 mL of brine. The mixture was dried over anhydrous sodium sulfate and concentrated. The residue was applied onto a silica gel column with dichloromethane/methanol (15:1). The crude product was purified by Prep-HPLC with the following conditions: column, X-Bridge Prep C18 19*150 mm Sum; mobile phase, A: water (it contains 10 mM NH₄HCO₃ 0.05% ammonia); B: ACN; Gradient: 20-45% B in 8 min; Flow rate: 20 mL/min; detector, UV 220 nm. The collected solution was concentrated under vacuum to remove CH₃CN and the resulting solution was dried by lyophilization. This resulted in 3.6 mg of N-[5-([6-[3-(hydroxymethyl)-2-[6-oxo-8-thia-5-azatricyclo[7.4.0.0\hat{ }[2,7]]trideca-1(9), 2(7)-dien-5-yl] pyridin-4-yl]-4-methyl-3-oxopyrazin-2-yl]amino)-2-[(2S)-2-methyl-4-(oxan-4-yl)piperazin-1-yl]phenyl]prop-2-enamide as a white solid. LC-MS-18 (ES, m/z): M+1: 765. ¹H NMR (300 MHz, DMSO-d₆, ppm) δ 9.21-9.26 (d, J=15.0 Hz, 2H), 9.11 (s, 1H), 8.47-8.49 (d, J=6.0 Hz, 1H), 7.91-7.93 (d, J=6.0 Hz, 1H), 7.72 (s, 1H), 7.58-7.61 (d, J=9.0 Hz, 1H), 7.24-7.26 (d, J=6.0 Hz, 1H), 6.56-6.66 (m, 1H), 6.27-6.32 (d, J=15.0 Hz, 1H), 5.79-5.83 (d, J=12.0 Hz, 1H), 4.93-4.95 (m, 1H), 4.50-4.60 (m, 1H), 4.03-4.21 (m, 1H), 3.86-3.97 (m, 2H), 3.56 (s, 3H), 3.29-3.30 (m, 4H), 3.16-3.18 (d, J=6.0 Hz, 1H), 2.90-3.02 (m, 4H), 2.73-2.80 (m, 4H), 2.51-2.73 (m, 4H), 1.71-1.90 (m, 6H), 1.45-1.47 (m, 2H), 1.24 (s, 1H), 0.73-0.75 (m, 3H).

### Compound 6A and 6B: Preparation of N-[5-([6-[3-(hydroxymethyl)-2-[6-oxo-8-thia-5-azatricyclo[7.4.0.0\hat{ }[2,7]]trideca-1(9), 2(7)-dien-5-yl]pyridin-4-yl]-4-methyl-3-oxopyrazin-2-yl]amino)-2-[(2R)-4-(oxan-4-yl)-2-(trifluoromethyl)piperazin-1-yl]phenyl]prop-2-enamide(Assumed) and N-[5-([6-[3-(hydroxymethyl)-2-[6-oxo-8-thia-5-azatricyclo[7.4.0.0\hat{ }[2,7]]trideca-1(9), 2(7)-dien-5-yl]pyridin-4-yl]-4-methyl-3-oxopyrazin-2-yl]amino)-2-[(2S)-4-(oxan-4-yl)-2-(trifluoromethyl)piperazin-1-yl]phenyl]prop-2-enamide(Assumed)

Synthesis of 2-(trifluoromethyl)pyrazine Into a 1 L 3-necked round-bottom flask purged and maintained with an inert atmosphere of nitrogen, was placed 2-iodopyrazine (20.00 g, 97.094 mmol, 1.00 equiv), DMSO (200.00 mL), CuI (3.70 g, 19.428 mmol, 0.20 equiv), 1,10-phenanthroline (3.50 g, 19.419 mmol, 0.2 equiv), KF (16.92 g, 291.239 mmol, 3.00 equiv), B(OMe)₃ (30.27 g, 291.282 mmol, 3.00 equiv), TMSCF₃ (41.42 g, 291.290 mmol, 3.00 equiv). The resulting solution was stirred for 2 h at 60 degrees C. in an oil bath. The resulting solution was diluted with 1 L of H₂O. The resulting solution was extracted with 3×150 mL of ethyl acetate. The resulting mixture was washed with 1×150 of H₂O. The resulting mixture was washed with 1×100 mL of NaCl. The mixture was dried over anhydrous sodium sulfate and concentrated carefully. The residue was applied onto a silica gel column and purified with ethyl acetate/petroleum ether (1:1). This resulted in crude of 2-(trifluoromethyl)pyrazine in 40 ml EA. LCMS-1: M+1: 149

Synthesis of 2-(trifluoromethyl)piperazine Into a 1-L pressure tank reactor, was placed 2-(trifluoromethyl)pyrazine (crude in 40 ml EA), MeOH (200 ml), PD/C (2.00 g). To the above H₂ (g) was introduced in. The resulting solution was stirred for 14 h at 60 degrees C. in an oil bath. The solids were filtered out. The resulting mixture was concentrated. This resulted in 4.0 g of 2-(trifluoromethyl)piperazine(crude) as a solid. LCMS-2: M+1: 155

Synthesis of tert-butyl 3-(trifluoromethyl)piperazine-1-carboxylate Into a 250-mL round-bottom flask, was placed 2-(trifluoromethyl)piperazine (4.00 g, crude), THF (100.00 mL), Boc₂O (8.50 g, 38.947 mmol, 1.50 equiv). The resulting solution was stirred for 14 h at room temperature. The resulting mixture was concentrated. The residue was applied onto a silica gel column and purified with ethyl acetate/petroleum ether (1:1). This resulted in 3.2 g (48.50%) of tert-butyl 3-(trifluoromethyl)piperazine-1-carboxylate as a white solid. LCMS-3: M+1: 254

Synthesis of tert-butyl 4-(4-nitrophenyl)-3-(trifluoromethyl)piperazine-1-carboxylate Into a 250-mL round-bottom flask purged and maintained with an inert atmosphere of nitrogen, was placed tert-butyl 3-(trifluoromethyl)piperazine-1-carboxylate (3.20 g, 12.586 mmol, 1.00 equiv), 4-bromo-1-nitrobenzene (5.08 g, 25.148 mmol, 2.00 equiv), 2G-Ad2n-BuP Pd(0.42 g, 20.629 mmol), Cs₂CO₃ (12.30 g, 37.751 mmol, 3.00 equiv), Toluene (100.00 mL). The resulting solution was stirred for 14 h at 105 degrees C. The resulting mixture was concentrated. The residue was applied onto a silica gel column and purified with ethyl acetate/petroleum ether (1:3). This resulted in 4 g (84.67%) of tert-butyl 4-(4-nitrophenyl)-3-(trifluoromethyl)piperazine-1-carboxylate as a brown solid. LCMS-4: M+1: 375

Synthesis of 1-(4-nitrophenyl)-2-(trifluoromethyl)piperazine Into a 250-mL round-bottom flask, was placed tert-butyl 4-(4-nitrophenyl)-3-(trifluoromethyl)piperazine-1-carboxylate (4.00 g), HCl in 1,4-dioxane (100.00 mL, 2M). The resulting solution was stirred for 14 h at room temperature. The resulting mixture was concentrated. The resulting solution was diluted with 100 mL of DCM. The resulting mixture was washed with 3×25 ml of NaHCO₃. The resulting mixture was washed with 1×25 mL of NaCl. The mixture was dried over anhydrous sodium sulfate. This resulted in 2.5 g of 1-(4-nitrophenyl)-2-(trifluoromethyl)piperazine as a brown solid. LCMS-5: M+1: 275

Synthesis of 1-(4-nitrophenyl)-4-(oxan-4-yl)-2-(trifluoromethyl)piperazine Into a 50-mL round-bottom flask, was placed 1-(4-nitrophenyl)-2-(trifluoromethyl)piperazine (1.50 g, 5.450 mmol, 1.00 equiv), tetrahydro-4H-pyran-4-one (1.09 g, 10.900 mmol, 2.00 equiv), DCE (20.00 mL), HOAc (0.10 mL, 0.002 mmol), NaBH(AcO)₃ (2.89 g, 13.636 mmol, 2.50 equiv). The resulting solution was stirred for 14 h at room temperature. The resulting mixture was concentrated. The residue was applied onto a silica gel column and eluted with ethyl acetate/petroleum ether (1:3). This resulted in 1.8 g (91.91%) of 1-(4-nitrophenyl)-4-(oxan-4-yl)-2-(trifluoromethyl)piperazine as a brown solid. LCMS-6: M+1: 360

Synthesis of 1-(2-bromo-4-nitrophenyl)-4-(oxan-4-yl)-2-(trifluoromethyl)piperazine Into a 50-mL round-bottom flask, was placed 1-(4-nitrophenyl)-4-(oxan-4-yl)-2-(trifluoromethyl)piperazine (1.80 g, 5.009 mmol, 1.00 equiv), TFA (20.00 mL), NBS (1.78 g, 10.018 mmol, 2.00 equiv). The resulting solution was stirred for 4 h at room temperature. The resulting solution was diluted with 100 mL of DCM. The resulting mixture was washed with 3×20 ml of NaHCO₃. The resulting mixture was washed with 1×20 mL of NaCl. The mixture was dried over anhydrous sodium sulfate. The residue was applied onto a silica gel column and eluted with ethyl acetate/petroleum ether (1:5). This resulted in 1.2 g (54.66%) of 1-(2-bromo-4-nitrophenyl)-4-(oxan-4-yl)-2-(trifluoromethyl)piperazine as a yellow solid. LCMS-7: M+1: 438, 440

Synthesis of tert-butyl N-[5-nitro-2-[4-(oxan-4-yl)-2-(trifluoromethyl)piperazin-1-yl] phenyl]carbamate Into a 50-mL round-bottom flask purged and maintained with an inert atmosphere of nitrogen, was placed 1-(2-bromo-4-nitrophenyl)-4-(oxan-4-yl)-2-(trifluoromethyl)piperazine (1.20 g, 2.738 mmol, 1.00 equiv), BocNH₂ (0.96 g, 8.215 mmol, 3.00 equiv), Toluene (20.00 mL), Xantphos Pd 2G (0.12 g, 0.135 mmol, 0.05 equiv), Cs₂CO₃ (2.68 g, 8.225 mmol, 3.00 equiv). The resulting solution was stirred for 2 h at 90 degrees C. in an oil bath. The resulting mixture was concentrated. The residue was applied onto a silica gel column and eluted with ethyl acetate/petroleum ether (1:1). This resulted in 1.1 g (84.67%) of tert-butyl N-[5-nitro-2-[4-(oxan-4-yl)-2-(trifluoromethyl)piperazin-1-yl]phenyl]carbamate as a brown solid. LCMS-8: M+1: 475

Synthesis of tert-butyl N-[5-amino-2-[4-(oxan-4-yl)-2-(trifluoromethyl)piperazin-1-yl] phenyl]carbamate Into a 50-mL round-bottom flask, was placed tert-butyl N-[5-nitro-2-[4-(oxan-4-yl)-2-(trifluoromethyl)piperazin-1-yl]phenyl]carbamate (1.10 g, 2.318 mmol, 1.00 equiv), MeOH (20.00 mL, 493.978 mmol, 213.08 equiv), Pd/C (0.17 g, 0.452 mmol, 0.19 equiv). To the above H₂ (g, 5 atm) was introduced in. The resulting solution was stirred for 14 h at room temperature. The solids were filtered out. The resulting mixture was concentrated. This resulted in 900 mg (87.34%) of tert-butyl N-[5-amino-2-[4-(oxan-4-yl)-2-(trifluoromethyl)piperazin-1-yl]phenyl]carbamate as a brown solid. LCMS-9: M+1: 445

Synthesis of tert-butyl (5-((6-bromo-4-methyl-3-oxo-3,4-dihydropyrazin-2-yl)amino)-2-(4-(tetrahydro-2H-pyran-4-yl)-2-(trifluoromethyl)piperazin-1-yl)phenyl)carbamate Into a 50-mL round-bottom flask purged and maintained with an inert atmosphere of nitrogen, was placed tert-butyl N-[5-amino-2-[4-(oxan-4-yl)-2-(trifluoromethyl)piperazin-1-yl]phenyl]carbamate (900.00 mg, 2.025 mmol, 1.00 equiv), 3,5-dibromo-1-methylpyrazin-2-one (813.67 mg, 3.037 mmol, 1.50 equiv), Pd-PEPPS^{IM}-IPent catalyst (160.50 mg, 0.202 mmol, 0.10 equiv), Cs₂CO₃ (1.98 g, 6.077 mmol, 3.00 equiv), Toluene (15.00 ml). The resulting solution was stirred for 14 h at 90 degrees C. in an oil bath. The resulting mixture was concentrated. The residue was applied onto a silica gel column and eluted with ethyl acetate/petroleum ether (1:1). This resulted in 450 mg of tert-butyl (5-((6-bromo-4-methyl-3-oxo-3,4-dihydropyrazin-2-yl)amino)-2-(4-(tetrahydro-2H-pyran-4-yl)-2-(trifluoromethyl)piperazin-1-yl)phenyl)carbamate as a brown solid. LCMS-10: M+1: 631, 633

Synthesis of 3-([3-amino-4-[4-(oxan-4-yl)-2-(trifluoromethyl)piperazin-1-yl]phenyl] amino)-5-bromo-1-methylpyrazin-2-one Into a 25-mL round-bottom flask, was placed tert-butyl N-[5-[(6-bromo-4-methyl-3-oxopyrazin-2-yl)amino]-2-[4-(oxan-4-yl)-2-(trifluoromethyl)piperazin-1-yl]phenyl]carbamate (350.00 mg, 1 equiv), DCM (6.00 mL), TFA (2.00 mL). The resulting solution was stirred for 2 h at room temperature. The resulting solution was diluted with 10 mL of DCM. The resulting mixture was washed with 3×10 ml of NaHCO₃. The resulting mixture was washed with 1×10 mL of NaCl. The mixture was dried over anhydrous sodium sulfate and concentrated. This resulted in 250 mg of 3-([3-amino-4-[4-(oxan-4-yl)-2-(trifluoromethyl)piperazin-1-yl]phenyl]amino)-5-bromo-1-methylpyrazin-2-one as a brown solid. LCMS-11: M+1: 531, 533

Synthesis of N-[5-[(6-bromo-4-methyl-3-oxopyrazin-2-yl)amino]-2-[4-(oxan-4-yl)-2-(trifluoromethyl)piperazin-1-yl]phenyl]prop-2-enamide Into a 8-mL vial, was placed 3-([3-amino-4-[4-(oxan-4-yl)-2-(trifluoromethyl)piperazin-1-yl] phenyl]amino)-5-bromo-1-methylpyrazin-2-one (240.00 mg, 0.452 mmol, 1.00 equiv), DCM (5.00 mL), TEA (68.55 mg, 0.677 mmol, 1.50 equiv), acryloyl chloride (44.97 mg, 0.497 mmol, 1.10 equiv). The resulting solution was stirred for 1 h at 0 degrees C. in a water/ice bath. The reaction was then quenched by the addition of 0.1 mL of MeOH. The resulting mixture was concentrated. The residue was applied onto a silica gel column and eluted with dichloromethane/methanol (100:5). This resulted in 220 mg (83.20%) of N-[5-[(6-bromo-4-methyl-3-oxopyrazin-2-yl)amino]-2-[4-(oxan-4-yl)-2-(trifluoromethyl)piperazin-1-yl]phenyl]prop-2-enamide as a brown solid. LCMS-12: M+1: 585, 587

Synthesis of 2, 4-dibromopyridine-3-carbaldehyde Into a 1000-mL 3-necked round-bottom flask, was placed 2, 4-dibromopyridine (40.00 g, 168.852 mmol, 1.00 equiv), THF (400.00 mL). This was followed by the addition of LDA (2M in hexane, 126.60 mL, 1.50 equiv) dropwise with stirring at −78 degrees C. The resulting solution was stirred for 1 h at −78 degrees C. Then DMF (16.04 ml, 219.507 mmol, 1.30 equiv) dropwise with stirring at −78 degrees C. The resulting solution was stirred for 0.5 h at −78 degrees C. The reaction was then quenched by the addition of 500 mL of NH₄Cl. The resulting solution was extracted with 3×500 mL of ethyl acetate concentrated. The residue was applied onto a silica gel column and eluted with ethyl acetate/petroleum ether (0:1-1:1). This resulted in 24.4 g (54.55%) of 2, 4-dibromopyridine-3-carbaldehyde as a white solid. LCMS-13: M+1: 264.

Synthesis of (2, 4-dibromopyridin-3-yl) methanol Into a 100-mL round-bottom flask, was placed 2, 4-dibromopyridine-3-carbaldehyde (2.00 g, 7.550 mmol, 1.00 equiv), EtOH (30.00 mL). This was followed by the addition of NaBH₄ (285.64 mg, 7.550 mmol, 1 equiv), in portions at 0 degrees C. The resulting solution was stirred for 3 h at room temperature. The reaction was then quenched by the addition of 30 mL of water. The resulting solution was extracted with 3×30 mL of ethyl acetate concentrated. The residue was applied onto a silica gel column and eluted with ethyl acetate/petroleum ether (1:1). The collected fractions were combined and concentrated. This resulted in 1.4 g (69.47%) of (2, 4-dibromopyridin-3-yl) methanol as a light yellow solid. LCMS-14: M+1: 266.

Synthesis of 2,4-dibromo-3-[(oxan-2-yloxy) methyl] pyridine Into a 100-mL round-bottom flask, was placed (2, 4-dibromopyridin-3-yl) methanol (1.40 g, 5.245 mmol, 1.00 equiv), DCM (30.00 mL, 0.353 mmol, 0.07 equiv), PPTS (131.81 mg, 0.525 mmol, 0.10 equiv), DHP (661.79 mg, 7.868 mmol, 1.50 equiv). The resulting solution was stirred for overnight at 45 degrees C. in an oil bath. The reaction was then quenched by the addition of 30 mL of water. The resulting solution was extracted with 3×30 mL of dichloromethane concentrated. The residue was applied onto a silica gel column and eluted with ethyl acetate/petroleum ether (1:1). The collected fractions were combined and concentrated. This resulted in 1.5 g of 2,4-dibromo-3-[(oxan-2-yloxy) methyl] pyridine as colorless oil. LCMS-15: M+1: 350.

Synthesis of N-(methoxymethyl)-N-methyl-4, 5, 6, 7-tetrahydro-1-benzothiophene-2-carboxamide Into a 250-mL 3-necked round-bottom flask purged and maintained with an inert atmosphere of nitrogen, was placed 4,5,6,7-tetrahydro-1-benzothiophene-2-carboxylic acid (8.0 g, 43.95 mmol, 1.0 equiv), DMF (193 mg, 2.197 mmol, 0.05 equiv), DCM (150 ml). This was followed by the addition of oxalyl chloride (6.1 g, 48.35 mmol, 1.1 equiv) dropwise with stirring at 0 degrees C. The resulting solution was stirred for 1 h in a water/ice bath. The mixture was concentrated. The crude product was dissolved in DCM (5 ml). To this was added TEA (13.3 g, 131.85 mmol, 3.0 equiv) and N,O-dimethylhydroxylamine HCl salt (4.3 g, 43.95 mmol, 1.0 equiv) at 0 degrees C. The resulting solution was stirred for 2 h at room temperature. The resulting solution was diluted with 100 mL of water. The resulting solution was extracted with 3×150 mL of dichloromethane and the organic layers combined. The resulting mixture was washed with 2×100 ml of water and 1×100 mL of brine. The mixture was dried over anhydrous sodium sulfate and concentrated. The residue was applied onto a silica gel column with ethyl acetate/petroleum ether (1:10). This resulted in 9.0 g of N-(methoxymethyl)-N-methyl-4, 5, 6, 7-tetrahydro-1-benzothiophene-2-carboxamide as a white solid. LCMS-16: M+1: 226.

Synthesis of 3-chloro-1-(4, 5, 6, 7-tetrahydro-1-benzothiophen-2-yl) propan-1-one Into a 250-mL 3-necked round-bottom flask purged and maintained with an inert atmosphere of nitrogen, was placed N-methoxy-N-methyl-4,5,6,7-tetrahydro-1-benzothiophene-2-carboxamide (8.00 g, 35.560 mmol, 1.00 equiv), THF (40.00 mL). This was followed by the addition of bromo(ethenyl)magnesium (1M in THF) (160.00 mL, 142.220 mmol, 4.00 equiv) dropwise with stirring at −10 degrees C. The resulting solution was stirred for 3 h at 0 degrees C. in an ice/salt bath. The reaction was then quenched by the addition of 40 mL of 2M HCl (aq). The resulting solution was extracted with 2×100 mL of ethyl acetate and the organic layers combined. The resulting mixture was washed with 2×100 ml of water and 1×100 mL of brine. The mixture was dried over anhydrous sodium sulfate and concentrated. The resulting solution was diluted with 80 mL of DCM. The residue was dissolved in 40 mL of 2M HCl(gas) in Et₂O. The resulting mixture was stirred for 3 h at R.T. Then the solution was concentrated. The residue was applied onto a silica gel column with ethyl acetate/petroleum ether (1:5). This resulted in 2.3 g of 3-chloro-1-(4, 5, 6, 7-tetrahydro-1-benzothiophen-2-yl) propan-1-one as yellow oil. LCMS-17: M+1: 229.

Synthesis of 1, 2, 5, 6, 7, 8-hexahydro-3H-benzo[b]cyclopenta[d]thiophen-3-one Into a 100-mL round-bottom flask, was placed 3-chloro-1-(4, 5, 6, 7-tetrahydro-1-benzothiophen-2-yl) propan-1-one (2.30 g, 10.090 mmol, 1.00 equiv), H₂SO₄ (20.00 mL). The resulting solution was stirred for 16 h at 95 degrees C. in an oil bath. The reaction mixture was cooled to room temperature with a water/ice bath. The resulting solution was diluted with 50 mL of water. The resulting solution was extracted with 2×50 mL of ethyl acetate and the organic layers combined. The resulting mixture was washed with 1×50 ml of brine. The mixture was dried over anhydrous sodium sulfate and concentrated. The residue was applied onto a silica gel column with ethyl acetate/petroleum ether (1:5). This resulted in 0.8 g of 1, 2, 5, 6, 7, 8-hexahydro-3H-benzo[b]cyclopenta[d]thiophen-3-one as brown oil. LCMS-18: M+1: 193.

Synthesis of (Z)-1, 2, 5, 6, 7, 8-hexahydro-3H-benzo[b]cyclopenta[d]thiophen-3-one oxime Into a 100-mL 3-necked round-bottom flask purged and maintained with an inert atmosphere of nitrogen, was placed NH₂OH·HCl (1.41 g, 20.313 mmol, 5.00 equiv), MeOH (30.00 mL). This was followed by the addition of NaOAc (1.66 g, 20.313 mmol, 5.00 equiv) at 0 degrees C. and the solution was stirred for 30 min at 0 degrees C. To this was added 1, 2, 5, 6, 7, 8-hexahydro-3H-benzo[b]cyclopenta[d]thiophen-3-one (780.00 mg, 4.063 mmol, 1.00 equiv) at 0 degrees C. The resulting solution was stirred for 18 h at room temperature. The resulting mixture was concentrated. The mixture was diluted with DCM (60 ml), then washed with water (2×30 ml) and brine (1×50 ml). The organic layers was combined and dried over anhydrous sodium sulfate and concentrated. The residue was applied onto a silica gel column with ethyl acetate/petroleum ether (1:1). This resulted in 300 mg of (Z)-1, 2, 5, 6, 7, 8-hexahydro-3H-benzo[b]cyclopenta[d]thiophen-3-one oxime as brown oil. LCMS-19: M+1: 208.

Synthesis of 3, 4, 5, 6, 7, 8-hexahydrobenzo [4, 5] thieno [2, 3-c] pyridin-1(2H)-one Into a 50-mL round-bottom flask purged and maintained with an inert atmosphere of nitrogen, was placed (Z)-1, 2, 5, 6, 7, 8-hexahydro-3H-benzo[b]cyclopenta[d]thiophen-3-one oxime (295.00 mg, 1.425 mmol, 1.00 equiv), PPA (6.00 mL). The resulting solution was stirred for 18 h at 80 degrees C. in an oil bath. The reaction mixture was cooled to room temperature with a water bath. The resulting solution was diluted with 20 mL of water. The mixture was extracted with 2×50 mL of ethyl acetate and the organic layers combined. The resulting mixture was washed with 1×50 ml of brine. The mixture was dried over anhydrous sodium sulfate and concentrated. The residue was applied onto a silica gel column with dichloromethane/methanol (5:1). This resulted in 260 mg of 3, 4, 5, 6, 7, 8-hexahydrobenzo [4, 5]thieno [2, 3-c] pyridin-1(2H)-one as an off-white solid. LCMS-20: M+1: 208. ¹H-NMR (300 MHz, DMSO-d₆, ppm) δ 3.37-3.43 (m, 2H), 2.73-2.76 (m, 2H), 2.61-2.65 (m, 2H), 2.44-2.48 (m, 2H), 1.74-1.80 (m, 4H).

Synthesis of 5-[4-bromo-3-[(oxan-2-yloxy) methyl] pyridin-2-yl]-8-thia-5-azatricyclo [7.4.0.0\hat{ }[2, 7]] trideca-1(9), 2 (7)-dien-6-one Into a 50-mL round-bottom flask purged and maintained with an inert atmosphere of nitrogen, was placed 8-thia-5-azatricyclo[7.4.0.0\hat{ }[2,7]]trideca-1(9),2(7)-dien-6-one (260.00 mg, 1.251 mmol, 1.00 equiv), 2,4-dibromo-3-[(oxan-2-yloxy)methyl]pyridine (873.00 mg, 1.875 mmol, 1.50 equiv), CuI (182.00 mg, 0.751 mmol, 0.60 equiv), Cs₂CO₃ (1.01 g, 2.502 mmol, 2.00 equiv), DMA (10.00 mL), 1,10-phenanthroline (182.00 mg, 0.751 mmol, 0.60 equiv). The resulting solution was stirred for 4 h at 110 degrees C. in an oil bath. The reaction mixture was cooled to room temperature with a water bath. The solids were filtered out. The resulting solution was diluted with 20 mL of water. The resulting solution was extracted with 2×20 mL of ethyl acetate and the organic layers combined. The resulting mixture was washed with 3×20 ml of water. The resulting mixture was washed with 1×20 mL of brine. The mixture was dried over anhydrous sodium sulfate and concentrated. The residue was applied onto a silica gel column with dichloromethane/methanol (10:1). This resulted in 360 mg of 5-[4-bromo-3-[(oxan-2-yloxy)methyl] pyridin-2-yl]-8-thia-5-azatricyclo[7.4.0.0\hat{ }[2,7]]trideca-1(9),2(7)-dien-6-one as dark brown oil. LCMS-21: M+1: 477/479.

Synthesis of 3-[(oxan-2-yloxy)methyl]-2-[6-oxo-8-thia-5-azatricyclo[7.4.0.0\hat{ }[2,7]]trideca-1(9), 2(7)-dien-5-yl]pyridin-4-ylboronic acid Into a 50-mL round-bottom flask purged and maintained with an inert atmosphere of nitrogen, was placed 5-[4-bromo-3-[(oxan-2-yloxy)methyl]pyridin-2-yl]-8-thia-5-azatricyclo[7.4.0.0\hat{ }[2,7]]trideca-1(9), 2(7)-dien-6-one (360.00 mg, 0.756 mmol, 1.00 equiv), bis(pinacolato)diboron (102.00 mg, 1.891 mmol, 2.50 equiv), KOAc (222.00 mg, 2.268 mmol, 3.00 equiv), Pd(dppf)Cl₂ (56.00 mg, 0.076 mmol, 0.10 equiv), Dioxane (20.00 mL). The resulting solution was stirred for 2 h at 100 degrees C. in an oil bath. The reaction mixture was cooled to room temperature with a water bath. The solids were filtered out. The resulting mixture was concentrated. The crude product was purified by Flash-Prep-HPLC with the following conditions (CombiFlash-1): Column, C18 silica gel; mobile phase, H₂O:ACN=20% increasing to H₂O:ACN=65% within 10 min; Detector, 220 nm. This resulted in 180 mg of 3-[(oxan-2-yloxy)methyl]-2-[6-oxo-8-thia-5-azatricyclo[7.4.0.0\hat{ }[2,7]]trideca-1(9),2(7)-dien-5-yl]pyridin-4-ylboronic acid as an off-white solid. LCMS-22: M+1: 443.

Synthesis of 5-[1-hydroxy-3H-[1, 2] oxaborolo [4, 3-c] pyridin-4-yl]-8-thia-5-azatricyclo [7.4.0.0\hat{ }[2, 7]] trideca-1(9), 2(7)-dien-6-one Into a 50-mL round-bottom flask, was placed 3-[(oxan-2-yloxy)methyl]-2-[6-oxo-8-thia-5-azatricyclo [7.4.0.0\hat{ }[2,7]]trideca-1(9),2(7)-dien-5-yl]pyridin-4-ylboronic acid (160.00 mg, 0.362 mmol, 1.00 equiv), 4N HCl in Dioxane (5.00 mL). The resulting solution was stirred for 1 h at room temperature. The solids were collected by filtration. The solids was washed by water 10 ml. This resulted in 100 mg of 5-[1-hydroxy-3H-[1,2]oxaborolo[4,3-c]pyridin-4-yl]-8-thia-5-azatricyclo[7.4.0.0\hat{ }[2,7]] trideca-1(9),2(7)-dien-6-one as an off-white solid. LCMS-23: M+1: 341.

Synthesis of N-[5-([6-[3-(hydroxymethyl)-2-[6-oxo-8-thia-5-azatricyclo[7.4.0.0\hat{ }[2,7]] trideca-1(9), 2(7)-dien-5-yl]pyridin-4-yl]-4-methyl-3-oxopyrazin-2-yl]amino)-2-[4-(oxan-4-yl)-2-(trifluoromethyl)piperazin-1-yl]phenyl]prop-2-enamide Into a 50-mL round-bottom flask purged and maintained with an inert atmosphere of nitrogen, was placed N-[5-[(6-bromo-4-methyl-3-oxopyrazin-2-yl)amino]-2-[4-(oxan-4-yl)-2-(trifluoromethyl) piperazin-1-yl]phenyl]prop-2-enamide (150.00 mg, 1.00 equiv), 5-[1-hydroxy-3H-[1,2] oxaborolo[4,3-c]pyridin-4-yl]-8-thia-5-azatricyclo[7.4.0.0\hat{ }[2,7]]trideca-1(9), 2(7)-dien-6-one (176.00 mg, 2.00 equiv), K₃PO₄ (180.00 mg, 3.00 equiv), Toluene (10.00 mL), H₂O (1.00 mL), BrettPhos Pd G3 (15.00 mg, 0.20 equiv). The resulting solution was stirred for 1 h at 90 degrees C. in an oil bath. The reaction mixture was cooled to room temperature with a water bath. The resulting solution was diluted with 50 mL of EtOAc. The solids were filtered out. The mixture was washed by water 20 ml*2 and brine 20 mL. The mixture was dried over anhydrous sodium sulfate and concentrated. The residue was applied onto a silica gel column with dichloromethane/methanol (20:1). This resulted in 60 mg of N-[5-([6-[3-(hydroxymethyl)-2-[6-oxo-8-thia-5-azatricyclo[7.4.0.0\hat{ }[2,7]]trideca-1(9),2(7)-dien-5-yl]pyridin-4-yl]-4-methyl-3-oxopyrazin-2-yl]amino)-2-[4-(oxan-4-yl)-2-(trifluoromethyl)piperazin-1-yl]phenyl]prop-2-enamide as a light brown solid. LCMS-24: M+1: 819.

Synthesis of N-[5-([6-[3-(hydroxymethyl)-2-[6-oxo-8-thia-5-azatricyclo[7.4.0.0\hat{ }[2,7]]trideca-1(9), 2(7)-dien-5-yl]pyridin-4-yl]-4-methyl-3-oxopyrazin-2-yl]amino)-2-[(2R)-4-(oxan-4-yl)-2-(trifluoromethyl)piperazin-1-yl]phenyl]prop-2-enamide(Assumed) The N-[5-([6-[3-(hydroxymethyl)-2-[6-oxo-8-thia-5-azatricyclo[7.4.0.0\hat{ }[2,7]]trideca-1(9),2(7)-dien-5-yl]pyridin-4-yl]-4-methyl-3-oxopyrazin-2-yl]amino)-2-[4-(oxan-4-yl)-2-(trifluoromethyl)piperazin-1-yl]phenyl]prop-2-enamide (58.00 mg, 0.071 mmol, 1.00 equiv, 85%) was purified by Chiral-Prep-HPLC with the following conditions: Column, CHIRALPAK ID-3,4.6*50 mm, 3 um, ID30CC-TE003; mobile phase A: Ethanol (0.1% DEA); mobile phase B: Acetonitrile; Flow rate: 1.0 ml/min; Gradient: 0% B to 30% B in 6 min; Detector, 220 nm. This resulted in 5 mg of N-[5-([6-[3-(hydroxymethyl)-2-[6-oxo-8-thia-5-azatricyclo [7.4.0.0\hat{ }[2,7]]trideca-1(9),2(7)-dien-5-yl]pyridin-4-yl]-4-methyl-3-oxopyrazin-2-yl]amino)-2-[(2R)-4-(oxan-4-yl)-2-(trifluoromethyl)piperazin-1-yl]phenyl]prop-2-enamide(Assumed, RT=2.47 min at CHIRALPAK ID-3) as a white solid. LCMS-0: M+1: 819; ee=99.99%. ¹H NMR (300 MHz, CD₃OD-d₄, ppm) δ 8.94 (s, 1H), 8.52-8.54 (d, J=6.0 Hz, 1H), 7.92 (s, 1H), 7.69 (s, 1H), 7.58-7.62 (dd, J=9.0, 6.0 Hz, 1H), 7.39-7.42 (d, J=9.0 Hz, 1H), 6.45-6.53 (m, 1H), 6.32-6.38 (d, J=12.0 Hz, 1H), 5.80-5.84 (d, J=12.0 Hz, 1H), 4.77-4.79 (m, 2H), 4.61-4.65 (m, 2H), 4.27-4.29 (m, 1H), 3.97-4.05 (m, 3H), 3.85 (s, 1H), 3.66 (s, 3H), 3.41-3.49 (m, 2H), 3.09-3.21 (m, 2H), 2.87-3.04 (m, 6H), 2.59-2.67 (m, 6H), 1.85-1.90 (m, 6H), 1.59-1.65 (m, 2H).

Synthesis of N-[5-([6-[3-(hydroxymethyl)-2-[6-oxo-8-thia-5-azatricyclo[7.4.0.0\hat{ }[2,7]]trideca-1(9), 2(7)-dien-5-yl]pyridin-4-yl]-4-methyl-3-oxopyrazin-2-yl]amino)-2-[(2S)-4-(oxan-4-yl)-2-(trifluoromethyl)piperazin-1-yl]phenyl]prop-2-enamide(Assumed) The N-[5-([6-[3-(hydroxymethyl)-2-[6-oxo-8-thia-5-azatricyclo[7.4.0.0\hat{ }[2,7]]trideca-1(9),2(7)-dien-5-yl]pyridin-4-yl]-4-methyl-3-oxopyrazin-2-yl]amino)-2-[4-(oxan-4-yl)-2-(trifluoromethyl)piperazin-1-yl]phenyl]prop-2-enamide (58.00 mg, 0.071 mmol, 1.00 equiv, 85%) was purified by Chiral-Prep-HPLC with the following conditions: Column, CHIRALPAK ID-3,4.6*50 mm, 3 um, ID30CC-TE003; mobile phase A: Ethanol (0.1% DEA); mobile phase B: Acetonitrile; Flow rate: 1.0 ml/min; Gradient: 0% B to 30% B in 6 min; Detector, 220 nm. This resulted in 5.1 mg of N-[5-([6-[3-(hydroxymethyl)-2-[6-oxo-8-thia-5-azatricyclo [7.4.0.0\hat{ }[2,7]]trideca-1(9),2(7)-dien-5-yl] pyridin-4-yl]-4-methyl-3-oxopyrazin-2-yl]amino)-2-[(2S)-4-(oxan-4-yl)-2-(trifluoromethyl)piperazin-1-yl]phenyl]prop-2-enamide(Assumed, RT=4.22 min at CHIRALPAK ID-3) as a white solid. LCMS-0: M+1: 819; ee=99.93%. ¹H NMR (300 MHz, CD₃OD-d₄, ppm) δ 8.94 (s, 1H), 8.52-8.54 (d, J=6.0 Hz, 1H), 7.92 (s, 1H), 7.69 (s, 1H), 7.58-7.62 (dd, J=9.0, 6.0 Hz, 1H), 7.39-7.42 (d, J=9.0 Hz, 1H), 6.45-6.53 (m, 1H), 6.32-6.38 (d, J=12.0 Hz, 1H), 5.80-5.84 (d, J=12.0 Hz, 1H), 4.77-4.79 (m, 2H), 4.61-4.65 (m, 2H), 4.27-4.29 (m, 1H), 3.97-4.05 (m, 3H), 3.85 (s, 1H), 3.66 (s, 3H), 3.41-3.49 (m, 2H), 3.09-3.21 (m, 2H), 2.87-3.04 (m, 6H), 2.59-2.67 (m, 6H), 1.85-1.90 (m, 6H), 1.59-1.65 (m, 2H).

In one embodiment, the BTK inhibitors disclosed herein are in free base form.

### Organic Acid

An organic acid is an organic compound with acidic properties. In one embodiment, the organic acid used in the tablet compositions disclosed herein is selected from citric acid, fumaric acid, maleic acid, acetic acid, succinic acid, and tartaric acid.

In one specific embodiment, the organic acid is fumaric acid. In one specific embodiment, the organic acid is citric acid. In one specific embodiment, the organic acid is maleic acid. In one specific embodiment, the organic acid is acetic acid. In one specific embodiment, the organic acid is succinic acid. In one specific embodiment, the organic acid is tartaric acid.

In certain embodiments, the organic acid is a mixture of one or more of citric acid, fumaric acid, maleic acid, acetic acid, succinic acid, and tartaric acid.

### Tablets

The compound disclosed herein (e.g., a compound of Formula (I), (II) or (III)) free base content in the tablet compositions is from about 5 mg to about 500 mg, from about 10 mg to about 250 mg, from about 20 mg to about 100 mg.

In some embodiments, the compound disclosed herein free base content in the tablet compositions is about 5 mg, about 10 mg, about 15 mg, about 20 mg, about 25 mg, about 50 mg, about 75 mg, about 100 mg, about 150 mg, about 200 mg, about 250 mg, about 300 mg, about 350 mg, about 400 mg, about 450 mg, about 500 mg, and ranges thereof, such as from about 25 mg to about 300 mg, from about 25 mg to about 200 mg, from about 25 mg to about 100 mg, from about 50 mg to about 150 mg, from about 100 mg to about 200 mg, from about 100 mg to about 300 mg, or from about 150 mg to about 250 mg.

Based on tablet weight, the compound free base content in the tablet composition is about 5 wt. %, about 10 wt. %, about 15 wt. %, about 20 wt. %, about 25 wt. %, about 30 wt. %, about 35 wt. % or about 40 wt. %, and ranges thereof, such as from about 5 wt. % to about 40 wt. %, from about 10 wt. % to about 40 wt. %, from about 15 wt. % to about 25 wt. %, from about 15 wt. % to about 30 wt. %, or from about 20 wt. % to about 25 wt. %.

The organic acid (e.g., citric acid, fumaric acid, maleic acid, acetic acid, succinic acid, or tartaric acid) content in the tablet compositions is from about 5 wt. % to about 50 wt. %, from about 5 wt. % to about 40 wt. %, from about 5 wt. % to about 30 wt. %, from about 10 wt. % to about 30 wt. %, from about 20 wt. % to about 25 wt. %, from about 5 wt. % to about 15 wt. %, or from about 10 wt. % to about 15 wt. %.

In some embodiments, the organic acid (e.g., fumaric acid) content in the tablet composition is about 5 wt. %, about 10 wt. %, about 15 wt. %, about 20 wt. %, about 25 wt. %, about 30 wt. %, about 35 wt. %, about 40 wt. %, about 45 wt. % or about 50 wt. %, and ranges thereof, such as from about 5 wt. % to about 50 wt. %, from about 5 wt. % to about 40 wt. %, from about 5 wt. % to about 30 wt. %, from about 5 wt. % to about 20 wt. %, from about 10 wt. % to about 30 wt. %, from about 15 wt. % to about 25 wt. %, from about 20 wt. % to about 25 wt. %, from about 5 wt. % to about 15 wt. %, or from about 10 wt. % to about 15 wt. %. In some other aspects, fumaric acid is present as an extra-granular component in the tablet. In some other aspects, fumaric acid is present as an intra-granular component in the tablet. In some other aspects, fumaric acid may be present as both and intra-granular component and as an extra-granular component.

In the tablet compositions, the weight ratio of the compound disclosed herein (e.g., a compound of Formula (I), (II) or (III)) to the organic acid (e.g., citric acid, fumaric acid, maleic acid, acetic acid, succinic acid, or tartaric acid) is from about 1:5 to about 5:1, is from about 1:4 to about 4:1, from about 1:3 to about 3:1, from about 1:2 to about 2:1, from about 1: 1.5 to about 1.5: 1, about 1:1, about 1:1.1, about 1:1.2, about 1:1.25, about 1:1.3, about 1:1.4, or about 1:1.5.

The tablet weight is about 50 mg, about 100 mg, about 200 mg, about 300 mg, about 400 mg, about 500 mg, about 600 mg, about 700 mg, about 800 mg, about 900 mg, about 1000 mg, or 1100 mg, or about 1200 mg.

The tablet compositions of the present disclosure may further suitably comprise one or more pharmaceutically acceptable excipients selected from, but not limited to fillers (diluents), binders, disintegrants, lubricants, and glidants.

A filler (or diluent) may be used to increase the bulk volume of the powdered drug making up the tablet. A binder may be used to ensure that granules and tablets can be formed with the required mechanical strength and hold a tablet together after it has been compressed, preventing it from breaking down into its component powders during packaging, shipping and routine handling. A disintegrant may be used to encourage the tablet to break down into small fragments, ideally individual drug particles, when it is ingested and thereby promote the rapid dissolution and absorption of drug. A lubricant may be used to ensure that the tableting powder does not adhere to the equipment used to press the tablet during manufacture, to improve the flow of the powder during mixing and pressing, and to minimize friction and breakage as the finished tablets are ejected from the equipment. A glidant may be used to improve the flowability of the powder making up the tablet during production.

Fillers and binders may include calcium hydrogenphosphate, microcrystalline cellulose (Avicel®), lactose, or any other suitable bulking agent. Examples of suitable fillers include microcrystalline cellulose, such as Avicel PH 101, Avicel PHI 02, Avicel PH 200, Avicel PH 105, Avicel DG, Ceolus KG 802, Ceolus KG 1000, SMCCSO and Vivapur 200; lactose monohydrate, such as Lactose FastFlo; microcrystalline cellulose co-processed with other excipients, such as microcrystalline cellulose coprocessed with lactose mono hydrate (MicroceLac 100) and microcrystalline cellulose co-processed with colloidal silicon dioxide (SMCCSO, Prosolv 50 and Prosolv HD 90); mixtures of isomaltulose derivatives such as galenlQ; and other suitable fillers and combinations thereof. The filler may be present as an intra-granular component and/or as an extra-granular component.

In some specific embodiments, the tablet compositions of the present disclosure comprise lactose and microcrystalline cellulose.

Disintegrants may be included in the disclosed formulations to promote separation of the granules within the compact from one another and to maintain separation of the liberated granules from one another. Distintegrants may be present as an intra-granular component and/or as an extra-granular component. Disintegrants may include any suitable disintegrant such as, for example, crosslinked polymers such as cross-linked polyvinyl pyrrolidone and cross-linked sodium carboxymethylcellulose or croscarmellose sodium. In some particular aspects, the disintegrant is croscarmellose sodium. The disintegrant content is suitably about 1 wt. %, about 1.5 wt. %, about 2 wt. %, about 2.5 wt. %, about 3 wt. %, about 3.5 wt. %, about 4 wt. %, about 4.5 wt. %, or about 5 wt. %, and ranges thereof, such as from about 1 wt. % to about 5 wt. %, or from about 2 wt. % to about 4 wt. %.

Lubricants may be used in compacting granules in the pharmaceutical composition. Lubricants may include, for example, polyethylene glycol (e.g., having a molecular weight of from about 1000 to about 6000), magnesium and calcium stearates, sodium stearyl fumarate, talc, or any other suitable lubricant. In some particular aspects, the lubricant is magnesium stearate and/or sodium stearyl fumarate. The lubricant may be present as an intra-granular component and/or as an extra-granular component. The lubricant content is suitably about 0.5 wt. %, about 1 wt. %, about 1.5 wt. %, about 2 wt. %, about 2.5 wt. %, about 3 wt. %, about 3.5 wt. %, about 4 wt. %, about 4.5 wt. %, or about 5 wt. %, and ranges thereof, such as from about 0.5 wt. % to about 5 wt. %, from about 1 wt. % to about 4 wt. %, from about 1 wt. % to about 3 wt. %, or from about 1 wt. % to about 2 wt. %.

Glidants may include, for example, colloidal silicon dioxide, including highly dispersed silica (Aerosil®), or any other suitable glidant such as animal or vegetable fats or waxes. In some particular aspects, the glidant is fumed silica. The glidant content is suitably about 0.1 wt. %, about 0.5 wt. %, about 1 wt. %, about 1.5 wt. %, about 2 wt. %, about 2.5 wt. % or about 3 wt. %, and ranges thereof, such as from about 0.1 wt. % to about 3 wt. %, from about 0.5 wt. % to about 2 wt. %, from about 0.5 wt. % to about 1.5 wt. %.

A coating, such as a film coating, may be applied to the tablets of the present disclosure. A film coat may be used to, for example, contribute to the ease with which the tablet can be swallowed. A film coat may also be employed to improve taste and appearance. If desired, the film coat may be an enteric coat. The film coat may comprise a polymeric film-forming material such as hydroxypropyl methylcellulose, hydroxypropyl cellulose, acrylate or methacrylate copolymers, and polyvinyl alcohol-polyethylene glycol graft copolymers such as Opadry and Kollicoat IR. In addition to a film-forming polymer, the film coat may further comprise a plasticizer, e.g. polyethylene glycol, a surfactant, e.g. a Tween® type, and optionally a pigment, e.g. titanium dioxide or iron oxides. The film-coating may also comprise talc as an anti-adhesive. The film coat typically accounts for less than about 5% by weight of the dosage form.

In some aspects of the disclosure, tablets may be prepared by a process comprising pre-blending, direct tablet compression, and coating. In some other aspects, tablets may be prepared by a process comprising (i) pre-blending, (ii) granulation and sizing, such as by roller compaction and milling or by dry granulation, (iii) blending/lubrication, (iv) tablet compression, and (v) coating.

Pre-blending is designed to provide substantial homogeneity of the intra-granular components prior to roller compaction. Pre-blending equipment and related process parameters that provide for essentially homogeneous blends are known to those skilled in the art. Suitable blenders are known in the art and any apparatus typically employed in the pharmaceutical industry for uniformly admixing two or more components including V-shaped blenders, double-cone blenders, bin (container) blenders, and rotary drum blenders. The combination blender volume, blender fill, rotation speed and rotation time may be suitably determined by those skilled in the art in order to achieve an essentially homogeneous admixture of components. Blender volume is suitably about 2 L, about 50 L, about 100 L, about 200 L, about 250 L, about 500 L, about 650 L or about 1000 L. Selection of blender fill allows for convection and three-dimensional material movement, and is suitably about 25%, about 30%, about 35%, about 40%, about 50%, about 60% or about 70%, and ranges thereof, such as from about 30% to about 60%, from about 45% to about 65%, from 32% to 53% or from 32% to 40%. Blend time is suitably, 5 min, 10 min, 15 min, 20 min, 30 min, 40 min, 50 min, 60 min, or more. Rotation rate is suitably, for instance, 2 rpm, 3 rpm, 4 rpm, 5 rpm, 6 rpm, 7 rpm, 8 rpm, 9 rpm or 10 rpm.

Granulation and sizing may be achieved using any suitable method known to those skilled in the art. In some particular aspects of the disclosure, granulation and sizing comprises dry granulation, milling and screening (sieving). In some other aspects of the disclosure, dry granulation is roller compaction. Granulation and sizing improves flow and compression characteristics of the admixture of active drug and excipients. Roller compaction is a process wherein pre-blend powder particles are made to adhere together resulting in larger, granular multi-particle entities. Roller compaction generally comprises three unit operations including a feeding system, a compaction unit and a milling/sieving unit. In the compaction unit, the pre-blend is compacted between counter-rotating rolls by application of a roller compaction force (expressed in kN/cm) to form a formed mass of compacted material, such as a ribbon or a sheet. The distance between the rolls is defined as the gap width. The formed ribbon of compacted material is processed in a size reduction unit by milling to form granules that are screened to produce a plurality of granules having a desired particle size distribution.

Roller compaction and milling equipment is available commercially from a number of manufacturers including Gerteis, Fitzpatrick® and Freund-Vector. Such equipment generally provides for control of roller compaction force, gap width, roller speed and feed rate. The roller surfaces may be smooth, knurled, or one roller surface may be smooth and the other roller surface may be knurled. In any of the various aspects, the pre-blend is charged to a roller compactor feed hopper. Roller compaction is performed at a specified force and gap size, and the process is preferably run under gap control. In any of the various aspects of the disclosure, the gap size is about 2 mm, about 3 mm, about 4 mm or about 5 mm, or more, and ranges thereof, such as from about 2 mm to about 5 mm, from about 2 mm to about 4 mm, from about 3 mm to about 5 mm or from about 4 mm to about 5 mm. The roller compaction force is about 1 kN/cm, about 2 kN/cm, about 3 kN/cm, about 4 kN/cm, about 5 kN/cm, about 6 kN/cm, about 7 kN/cm or about 8 kN/cm, or more, and ranges thereof, such as from about 1 kN/cm to about 8 kN/cm, from about 2 kN/cm to about 5 kN/cm or from about 2 kN/cm to about 4 kN/cm. The formed ribbons or sheet may be milled through a screen to produce granules. In some aspects of the disclosure, the screen is integral to the mill. In any of the various aspects of the disclosure, the milling screen size is 0.5 mm, 0.75 mm, 1.0 mm, 1.25 mm, 1.5 mm, 1.75 mm, 2.0 mm, 2.25 mm or 2.5 mm, and ranges thereof, such as from about 0.5 mm to about 2.5 mm, from about 0.5 mm to about 2.0 mm, from about 0.5 mm to about 1.5 mm, from about 0.5 mm to about 1.25 mm, from about 0.75 mm to about 2.5 mm, from about 0.75 mm to about 2.0 mm, from about 0.75 mm to about 1.5 mm, or from about 0.75 mm to about 1.25 mm.

In the final blending step, granules formed by roller compaction and milling are charged to a blender and any extra-granular component, such as disintegrant (e.g., croscarmellose sodium) and lubricant (e.g., magnesium stearate or sodium stearyl fumarate), and optionally organic acid (e.g., fumaric acid), is added to the blender to form an admixture. The final blending step provides for an essentially homogeneous distribution of any external disintegrant and lubricant and provides for acceptable processability during tablet compression. Suitable blenders and related process variables are described above.

Filler, lubricant and disintegrants are typically delumped by screening prior to blending. Screening methods are known to this skilled in the art. In an example of one particular pre-blend aspect of the disclosure, filler (e.g. lactose monohydrate and MCC) and disintegrant (e.g., croscarmellose sodium) are delumped by screening and are combined with compound (I) in a blender, and the blender contents are blended for a blend time (e.g., 30 minutes) at a fixed rotation rate (e.g., 6 rpm). Lubricant (e.g., magnesium stearate) is delumped by screening and is added to a blender containing admixed filler, disintegrant and compound (I). The blender contents are blended for a blend time (e.g., 2 minutes to 30 minutes) at a fixed rotation rate (e.g., 5 rpm to 10 rpm) to form the pre-blend.

In the tableting step, a tableting die mold is filled with final blend material and the mixture is compressed to form a tablet core that is ejected. Suitable tablet presses are known in the art and are available commercially from, for instance, Riva-Piccola, Carver, Fette, Bosch Packaging Technology, GEA and Natoli Engineering Company. Generally, each tablet is made by pressing the granules inside a die, made up of hardened steel. The die is typically a disc shape with a hole cut through its center. The powder is compressed in the center of the die by two hardened steel punches that fit into the top and bottom of the die thereby forming the tablet. Tablet compression may be done in two stages with the first, pre-compression, stage involving tamping down the powder and compacting the blend slightly prior to application of the main compression force for tablet formation. The tablet is ejected from the die after compression.

Main compression force affects tablet characteristics such as hardness and appearance. Main compression force further has an impact on sticking of the final blend to tablet tooling during compression, with increased force leading to reduced sticking and, hence, fewer tablets with appearance defects. Further, the compressibility of the final blend can impact the quality (such as the presence or lack of defects) of the resultant tablet core.

Compression processing parameters, such as compression force and run time, can also have an impact. In some aspects of the disclosure, the compression force is about 5 kN, about 6 kN, about 7 kN, about 8 kN, about 9 kN, about 10 kN, about 11 kN, about 12 kN, about 13 kN, about 14 kN, about 15 kN, about 16 kN, about 17 kN, about 18 kN, about 19 kN, about 20 kN, or more, and ranges thereof, such as from about 5 kN to about 20 kN, from about 14 kN to about 19 kN, from about 14 kN to about 18 kN, or from about 8 kN to about 13 kN.

The tablet cores may be film-coated to ensure that tablets are essentially tasteless and odorless, and are easy to swallow. Film coating also prevents dust formation during packaging and ensures robustness during transportation. Film coating may suitably be done by methods known in the art such as by pan coating. Suitable coating equipment includes, without limitation, a Glatt GC1000S.

In some aspects of the disclosure, tablet cores are charged to a coating pan and warmed to a target temperature. The coating suspension is prepared to a target solids content. Once the tablets are within the target temperature range, drum rotation and spraying are runs at target rates designed to achieve predetermined weight gain of about 3 wt. %, about 4 wt. % or about 5 wt. %. Outlet air temperature is maintained in a range to ensure that the target product temperature is obtained throughout coating. Once spraying is complete, the coated tablets are dried and cooled down before discharging the film-coated tablets. A solid content of a coating suspension is suitably from about 10 wt. % to about 20 wt. %, or from about 15 wt. % to about 20 wt. %. The coating spray rate per kg of tablet cores is suitably about 0.5 g/min to about 2.5 g/min, or from about 1 g/min to about 2 g/min. The coating temperature is suitably from about 30° C. to about 60° C., or from about 40° C. to about 50° C. The pan rotational speed is suitably from about 2 to about 20 rpm, from about 4 to about 15 rpm, or from about 8 to about 12 rpm. The inlet air volume varies with the batch size and is suitably from about 300 to about 1500 m³/h, from about 450 to about 1200 m³/h, or from about 1000 to about 1250 m³/h.

### Methods of Treatment

The present disclosure further provides methods for the prevention or treatment of a neoplastic disease, autoimmune and/or inflammatory disease. In one embodiment, the invention relates to a method of treating a neoplastic disease, autoimmune and/or inflammatory disease in a subject in need of treatment comprising administering to said subject a therapeutically effective amount of a compound of the invention. In one embodiment, the invention further provides for the use of a compound of the invention in the manufacture of a medicament for halting or decreasing a neoplastic disease, autoimmune and/or inflammatory disease.

In one embodiment, the neoplastic disease is a B-cell malignancy includes but not limited to B-cell lymphoma, lymphoma (including Hodgkin's lymphoma and non-Hodgkin's lymphoma), hairy cell lymphoma, small lymphocytic lymphoma (SLL), mantle cell lymphoma (MCL), and diffuse large B-cell lymphoma (DLBCL), multiple myeloma, chronic and acute myelogenous leukemia and chronic and acute lymphocytic leukemia.

The autoimmune and/or inflammatory diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barré syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and wegener's granulomatosis.

The dosage form compositions of the present disclosure may be employed alone or in combination with an additional or second therapeutic agent for the treatment of a disease or disorder described herein, such as inflammation or a hyperproliferative disorder (e.g., cancer). The additional therapeutic may be an anti-inflammatory agent, an immunomodulatory agent, chemotherapeutic agent, an apoptosis-enhancer, a neurotropic factor, an agent for treating cardiovascular disease, an agent for treating liver disease, an antiviral agent, an agent for treating blood disorders, an agent for treating diabetes, and an agent for treating immunodeficiency disorders. The second therapeutic agent may be an NSAID anti-inflammatory agent. The second therapeutic agent may be a chemotherapeutic agent. The second or additional therapeutic agent preferably has complementary activities to the compound of the invention, such that they do not adversely affect each other. Such compounds are suitably present in combination in amounts that are effective for the purpose intended.

The combination therapy may be administered in a simultaneous or in a sequential regimen. When administered sequentially, the combination may be dosed in two or more administrations. The combined administration includes co-administration, using separate formulations or a single pharmaceutical formulation, and consecutive administration in either order, wherein preferably there is a time period while both (or all) active agents simultaneously exert their biological activities. Suitable dosages for any of the above coadministered agents are those presently used and may be lowered due to the combined action (synergy) of the additional therapeutic agents.

The combination therapy may be synergistic such that the effect achieved when the active ingredients used together is greater than the sum of the effects that results from using the compounds separately. A synergistic effect may be attained when the active ingredients are: (1) administered or delivered simultaneously; (2) administered in alternation or in parallel; or (3) by some other regimen. When delivered in alternation therapy, a synergistic effect may be attained when the compounds are administered or delivered sequentially. In general, during alternation therapy, an effective dosage of each active ingredient is administered sequentially, i.e., serially, whereas in combination therapy, effective dosages of two or more active ingredients are administered together.

In combination therapy, a kit may comprise (a) a first container with a dosage form composition of the present disclosure and, optionally, (b) a second container with a second pharmaceutical formulation contained therein for co-administration with the dosage form compositions of the present disclosure. In such aspects, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

In certain embodiments, the method of treatment further comprises administering a second therapeutic agent effective to treat the cancer. The second therapeutic agent may comprise a chemotherapeuic agent, an iummunotherapeutic agent, radiation therapy, and/or surgery.

In certain embodiments, the chemotherapeutic agent comprises alkylating agents, antimetabolites, spindle poison plant alkaloids, cytotoxic/antitumor antibiotics, topoisomerase inhibitors, antibodies, photosensitizers, kinase inhibitors, or combination thereof.

In certain embodiments, the chemotherapeutic agent may include compounds used in “targeted therapy” and conventional chemotherapy. Examples of chemotherapeutic agents include: erlotinib, docetaxel, 5-FU (fluorouracil, 5-fluorouracil, CAS No. 51-21-8), gemcitabine, PD-0325901 (CAS No. 391210-10-9), cisplatin (cis-diamine, dichloroplatinum (II), CAS No. 15663-27-1), carboplatin (CAS No. 41575-94-4), paclitaxel, trastuzumab, temozolomide (4-methyl-5-oxo-2,3,4,6,8-pentazabicyclo[4.3.0]nona-2,7,9-triene-9-carboxamide, CAS No. 85622-93-1), tamoxifen ((Z)-2-[4-(1,2-diphenylbut-1-enyl)phenoxy]-N,N-dimethylethanamine), and doxorubicin, Akti-1/2, HPPD, and rapamycin.

In certain embodiments, the chemotherapeutic agent may include: oxaliplatin, bortezomib, sutent, letrozole, imatinib mesylate, XL-518 (Mek inhibitor, see WO 2007/044515), ARRY-886 (Mek inhibitor, AZD6244), SF-1126 (PI3K inhibitor), BEZ-235 (PI3K inhibitor), XL-147 (PI3K inhibitor), PTK787/ZK 222584, fulvestrant, leucovorin (folinic acid), rapamycin (sirolimus), lapatinib, lonafarnib, sorafenib, gefitinib, irinotecan, tipifarnib, ABRAXANE™ (Cremophor-free), albumin-engineered nanoparticle formulations of paclitaxel, vandetanib, chloranmbucil, AG1478, AG1571 (SU 5271), temsirolimus, pazopanib, canfosfamide, thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, calicheamicin gammall, calicheamicin omegall (Angew Chem. Intl. Ed. Engl. (1994) 33: 183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spiro germanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine; novantrone; teniposide; edatrexate; daunomycin; aminopterin; capecitabine; ibandronate; CPT-11; topoisomerase inhibitor RFS 2000; difluoromethylornithine (DMFO); retinoids such as retinoic acid; and pharmaceutically acceptable salts, acids and derivatives of any of the above.

In certain embodiments, chemotherapeutic agent includes: (i) anti-hormonal agents that act to regulate or inhibit hormone action on tumors such as anti-estrogens and selective estrogen receptor modulators (SERMs), including, for example, tamoxifen (including tamoxifen citrate), raloxifene, droloxifene, 4-hydroxytamoxifen, trioxifene, keoxifene, LY117018, onapristone, and toremifine citrate); (ii) aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab; and pharmaceutically acceptable salts, acids and derivatives of any of the above.

In certain embodiments, the chemotherapeutic agent include therapeutic antibodies such as alemtuzumab (Campath), bevacizumab; cetuximab; panitumumab, rituximab, pertuzumab, trastuzumab, tositumomab, and the antibody drug conjugate, gemtuzumab ozogamicin.

### EXAMPLES

The following examples are merely illustrative, and do not limit this disclosure in any way. For example, it will be appreciate that lab-scale compositions or formulations, or extrusion blends, referenced herein may in general be scaled up in view of the details provided without departing from the intended scope of the present application.

In the examples, “API” (active pharmaceutical ingredient) can be any compound of Formula A, added in essentially anhydrous parent-compound (i.e., not salt) form.

### Example 1: Dissolution of Compound Free Base Versus pH

The solubility of a typical API (i.e., Compound 2 as disclosed herein) in free base form was evaluated in buffers of varying pH. The results are reported in Table below.

| Sample ID | Ave. Conc.(mg/ml) |
|---|---|
| 0.01N HCl | 55.52 |
| pH 2.0 | 9.67 |
| pH 3.0 | 2.07 |
| pH 4.5 | 1.81 |
| pH 6.8 | 0.02 |
| Water | 0.09 |

### Example 2: Stability Study

The stability of a Compound 2 HCl salt at different temperatures for 8 hours is shown below. The initial purity at TO is 96.5%. The data suggest that, despite the enhanced solubility, the HCl salt of Compound 2 is not sufficiently stable, and is thus not good for further formulation development. Similar observation was also made for several other compounds of the invention.

| Compound-2 HCl | Purity (%) at 8 h |
|---|---|
| 50° C. | 95.7% |
| 80° C. | 90.1% |
| 100° C. | 84.7% |

Meanwhile, the stability of a physical mixture of Compound-2 in free base form with an organic acid—fumaric acid—under the condition of 40° C. and 75% RH (relative humidity) for 2 weeks is shown below. The data suggest that the mixture is surprisingly stable and is suitable for formulation development.

| A mixture of Compound 2 and | |
|---|---|
| Fumaric acid | Purity (%) |
| 0 | 99.1 |
| 2 weeks | 98.9 |

The observation that organic acid mixture with the compounds of the invention is surprisingly stable is also illustrated by the stability of a physical mixture of Compound 2 in free base form with citric acid monohydrate under the condition of 40° C. and 75% RH for 2 weeks, see below. The data suggest that the citric acid mixture is also sufficiently stable and suitable for formulation development.

| A mixture of Compound 2and | |
|---|---|
| Citric acid monohydrate | Purity (%) |
| 0 | 99.2 |
| 2 weeks | 99.7 |

The stability of a physical mixture of Compound 2 with succinic acid under the condition of 40° C. and 75% RH for 2 weeks is also shown below. The data suggest that mixture is good for formulation development.

| A mixture of Compound 2and | |
|---|---|
| Succinic acid | Purity (%) |
| 0 | 99.8 |
| 2 weeks | 100.4 |

### Example 3: The Preparation, Dissolution, and Dog PK Study of F47

The components in tablet F47 are listed in the table below wherein API is Compound 2:

| Component | | Vendor | Tablet(mg) | % |
|---|---|---|---|---|
| intra- | API in free base form | In House | 100.00 | 20.00 |
| granular | Fumaric acid | Sgima | 100.00 | 20.00 |
| | Microcrystalline | Dupont | 210.00 | 42.00 |
| | cellulose, | | | |
| | Avicel ® PH-102 | | | |
| | Croscarmellose | Dupont | 15.00 | 3.00 |
| | sodium, | | | |
| | Ac-Di-Sol ® | | | |
| | Magnesium stearate | Peter Greven | 5.00 | 1.00 |
| | Ligamed ® MF-2-V | | | |
| | Sub-total | | 430.00 | 86.00 |
| extra- | Microcrystalline | Dupont | 50.00 | 10.00 |
| granular | cellulose | | | |
| | Avicel ® PH-102 | | | |
| | Croscarmellose | Dupont | 15.00 | 3.00 |
| | sodium, | | | |
| | Ac-Di-Sol ® | | | |
| | Magnesium stearate | Peter Greven | 5.00 | 1.00 |
| | Ligamed ® MF-2-V | | | |

| | | |
|---|---|---|
| Total | 500.00 | 100.00 |

The tablet F47 were prepared as follows:

| | |
|---|---|
| 1 | Weigh prescribed amount of microcrystalline cellulose, |
| | croscarmellose sodium, magnesium stearate, fumaric acid and API, |
| | after passing through 30-mesh sieve, transfer to a mixing container |
| | and mix for 5 min; |
| 2 | Use φ12 mm round mold to press tablet, pressure 500 lbs, hardness |
| | 4 kg; |
| 3 | Crush the tablet from step 2 with Comil, use 30 mesh and 80 mesh |
| | screens for sizing, and re-granulate <80 mesh powder; |
| 4 | According to the yield, recalculate the additional amount of auxiliary |
| | materials. Weigh microcrystalline cellulose, croscarmellose sodium |
| | and magnesium stearate, and pass through a 30 mesh sieve; |
| 5 | Add microcrystalline cellulose and croscarmellose sodium to the |
| | granulated particles from step 3, after mixing for 5 minutes, add |
| | magnesium stearate, and then mix for additional 2 minutes; |
| 6 | Tablet Compression: use 15.3*8 mm oval shallow concave mold for |
| | compression, hardness 12 kg. |

Dissolution Test: The dissolution medium is 0.1 N HCl, a HCl solution with pH of 2, and a citrate buffer with pH of 3. The following tables (1), (2), and (3) show the results of the dissolution test.

(1) 0.1N HCl medium

| Time(min) | AVERAGE % | RSD %* |
|---|---|---|
| 0 | 0 | 0 |
| 5 | 83 | 15.7 |
| 10 | 86 | 13.3 |
| 15 | 88 | 10.8 |
| 20 | 88 | 10.5 |
| 30 | 89 | 9.5 |
| 45 | 89 | 8.4 |
| 60 | 94 | 1.3 |

RSD: relative standard deviation

(2) pH 2.0 HCl

| Time(min) | AVERAGE % | RSD % |
|---|---|---|
| 0 | 0 | 0 |
| 5 | 89 | 3.0 |
| 10 | 92 | 2.4 |
| 15 | 92 | 1.8 |
| 20 | 92 | 1.8 |
| 30 | 91 | 1.8 |
| 45 | 92 | 2.2 |
| 60 | 92 | 1.9 |

(1) pH 3.0 Citrate buffer

| Time(min) | AVERAGE % | RSD % |
|---|---|---|
| 0 | 0 | 0 |
| 5 | 67 | 4.4 |
| 10 | 79 | 0.8 |
| 15 | 85 | 1.9 |
| 20 | 88 | 2.9 |
| 30 | 90 | 2.5 |
| 45 | 90 | 3.0 |
| 60 | 90 | 4.0 |

The pharmacokinetics of tablet were evaluated in beage dog via oral administration. The oral doses were administered by gavage. The PK time point for PO arm was 15, 30 min, 1, 2, 4, 6, 8, 12, 24 hours post dose. Approximately 1.5 mL of blood was collected at each time point. Blood of each sample was transferred into plastic micro centrifuge tubes containing EDTA-K2, and plasma was collected within 15 min by centrifugation at 4000 g for 5 minutes in a 4° C. centrifuge. Plasma samples were stored in polypropylene tubes. The samples were stored in a freezer at −75±15° C. prior to analysis. Concentrations of compounds in the plasma samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data: IV administration: C₀, CL, V_{d}, T_{1/2}, AUC_{inf}, AUCₗₐₛₜ, MRT, Number of Points for Regression; PO administration: Cₘₐₓ, Tₘₐₓ, T_{1/2}, AUC_{inf}, AUCₗₐₛₜ, F %, Number of Points for Regression. The pharmacokinetic data was described using descriptive statistics such as mean, standard deviation. Additional pharmacokinetic or statistical analysis was performed at the discretion of the contributing scientist, and was documented in the data summary.

The dog PK of Tablet F47 (100 mg active API in the tablet) is shown below. The results indicate that this tablet shows satisfactory pharmacokinetic profile.

| | T½(h) | Tₘₐₓ(h) | Cₘₐₓ (ng/mL) | AUC₍₀₋ₜ₎(h*ng/ml) |
|---|---|---|---|---|
| F47, 100 mg | 2.7 | 1.2 | 4,420 | 22,400 |

### Example 4: The Preparation, Dissolution, and Dog PK Study of F48

The components in tablet F48 are listed in the table below wherein API is Compound 2:

| Component | | Vendor | Tablet(mg) | % |
|---|---|---|---|---|
| intra- | API in free base form | In House | 100.00 | 20.00 |
| granular | Fumaric acid | Sgima | 100.00 | 20.00 |
| | Microcrystalline | Dupont | 200.00 | 40.00 |
| | cellulose, | | | |
| | Avicel ® PH-102 | | | |
| | Croscarmellose | Dupont | 15.00 | 3.00 |
| | sodium, | | | |
| | Ac-Di-Sol ® | | | |
| | Magnesium stearate | Peter Greven | 5.00 | 1.00 |
| | Ligamed ® MF-2-V | | | |
| | Sodium dodecyl | BASF | 10.00 | 2.00 |
| | sulphate | | | |
| | Kolliphor SLS Fine | | | |
| | Sub-total | | 430.00 | 86.00 |
| extra- | Microcrystalline | Dupont | 50.00 | 10.00 |
| granular | cellulose | | | |
| | Avicel ® PH-102 | | | |
| | Croscarmellose | Dupont | 15.00 | 3.00 |
| | sodium, | | | |
| | Ac-Di-Sol ® | | | |
| | Magnesium stearate | Peter Greven | 5.00 | 1.00 |
| | Ligamed ® MF-2-V | | | |

| | | |
|---|---|---|
| Total | 500.00 | 100.00 |

The tablet F48 were prepared as follows:

| | |
|---|---|
| 1 | Weigh prescribed amount of microcrystalline cellulose, |
| | croscarmellose sodium, magnesium stearate, fumaric acid, API, and |
| | sodium dodecyl sulphate, after passing through 30-mesh sieve, |
| | transfer to a mixing container and mix for 5 min; |
| 2 | Use φ12 mm round mold to press tablet, pressure 500 lbs, hardness |
| | 4 kg; |
| 3 | Crush the tablet from step 2 with Comil, use 30 mesh and 80 mesh |
| | screens for sizing, and re-granulate <80 mesh powder; |
| 4 | According to the yield, recalculate the additional amount of auxiliary |
| | materials. Weigh microcrystalline cellulose, croscarmellose sodium |
| | and magnesium stearate, and pass through a 30 mesh sieve; |
| 5 | Add microcrystalline cellulose and croscarmellose sodium to the |
| | granulated particles from step 3, after mixing for 5 minutes, add |
| | magnesium stearate, and then mix for additional 2 minutes; |
| 6 | Tablet Compression: use 15.3*8 mm oval shallow concave mold for |
| | compression, hardness 12 kg. |

Dissolution Test: The dissolution medium is 0.1 N HCl, a HCl solution with pH of 2, and a citrate buffer with pH of 3. The following tables (1), (2), and (3) show the results of the dissolution test.

(3) 0.1N HCl medium

| Time(min) | AVERAGE % | RSD % |
|---|---|---|
| 0 | 0 | 0 |
| 5 | 53 | 4.2 |
| 10 | 63 | 3.7 |
| 15 | 67 | 3.5 |
| 20 | 69 | 3.3 |
| 30 | 71 | 3.5 |
| 45 | 73 | 2.5 |
| 60 | 82 | 1.3 |

(4) pH 2.0 HCl

| Time(min) | AVERAGE % | RSD % |
|---|---|---|
| 0 | 0 | 0 |
| 5 | 37 | 4.5 |
| 10 | 46 | 3.2 |
| 15 | 51 | 4.0 |
| 20 | 56 | 2.1 |
| 30 | 62 | 2.8 |
| 45 | 67 | 3.1 |
| 60 | 71 | 3.0 |

(2) pH 3.0 Citrate buffer

| Time(min) | AVERAGE % | RSD % |
|---|---|---|
| 0 | 0 | 0 |
| 5 | 17 | 1.0 |
| 10 | 26 | 5.5 |
| 15 | 32 | 6.1 |
| 20 | 43 | 26.6 |
| 30 | 44 | 9.1 |
| 45 | 50 | 6.9 |
| 60 | 55 | 5.5 |

The pharmacokinetics of tablet were evaluated in beage dog via oral administration. The oral doses were administered by gavage. The PK time point for PO arm was 15, 30 min, 1, 2, 4, 6, 8, 12, 24 hours post dose. Approximately 1.5 mL of blood was collected at each time point. Blood of each sample was transferred into plastic micro centrifuge tubes containing EDTA-K2, and plasma was collected within 15 min by centrifugation at 4000 g for 5 minutes in a 4° C. centrifuge. Plasma samples were stored in polypropylene tubes. The samples were stored in a freezer at −75±15° C. prior to analysis. Concentrations of compounds in the plasma samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data: IV administration: C₀, CL, V_{d}, T_{1/2}, AUC_{inf}, AUCₗₐₛₜ, MRT, Number of Points for Regression; PO administration: Cₘₐₓ, Tₘₐₓ, T_{1/2}, AUC_{inf}, AUCₗₐₛₜ, F %, Number of Points for Regression. The pharmacokinetic data was described using descriptive statistics such as mean, standard deviation. Additional pharmacokinetic or statistical analysis was performed at the discretion of the contributing scientist, and was documented in the data summary.

The dog PK of Tablet F48 (100 mg active API in the tablet) is shown below. The results indicate that F48 pharmacokinetic profile is not as good as that of F47.

| | T½(h) | Tₘₐₓ(h) | Cₘₐₓ(ng/mL) | AUC₍₀₋ₜ₎(h*ng/ml) |
|---|---|---|---|---|
| F48, 100 mg | 3.8 | 0.8 | 1,650 | 5,680 |

What is claimed is:

1. A tablet composition comprising an organic acid and a compound of Formula (I), or an N-oxide thereof, solvate, polymorph, tautomer, stereoisomer, an isotopic form, or a prodrug of said compound of Formula (I) or N-oxide thereof (e.g., in a physical mixture): 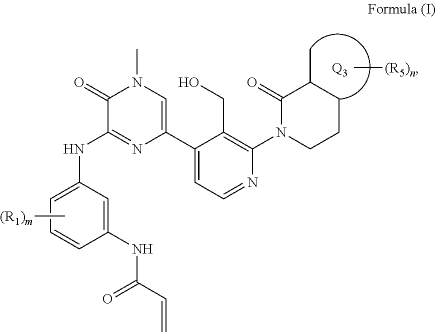
wherein
Q₃ is a 5-membered heteroaryl;
each of R₁ and R₅, independently, is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, aryl, heteroaryl is optionally substituted with one or more R_{d};
two of R₁ groups, taken together with the atom to which they are attached, may optionally form a cycloalkyl or heterocycloalkyl optionally substituted with one or more R_{d};
two of R₅ groups, taken together with the atom to which they are attached, may optionally form a cycloalkyl, heterocycloalkyl, aryl, or heteroaryl optionally substituted with one or more R_{d};
each of Rₐ, R_{b}, R_{c} and R_{d}, independently, is H, D, alkyl, spiroalkyl, alkenyl, alkynyl, halo, cyano, amine, nitro, hydroxy, ═O, —P(O)R_{b}R_{c}, -alkyl-P(O)R_{b}R_{c}, —S(O)(═N(R_{b}))R_{c}, —N═S(O)R_{b}R_{c}, ═NR_{b}, C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally substituted with one or more Rₑ;
each of Rₑ, independently, is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;
two of R_{d} groups, taken together with the atom to which they are attached, may optionally form a cycloalkyl or heterocycloalkyl optionally substituted with one or more Rₑ; and
each of m and n, independently, is 0, 1, 2, 3, or 4.

2. The tablet composition of claim 1, wherein the compound is represented by Formula (II): 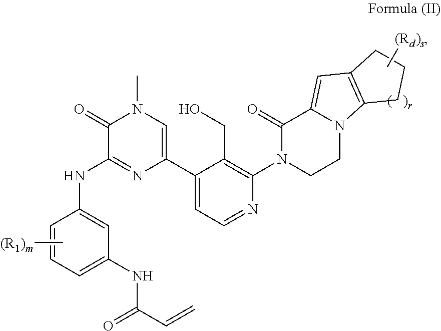
wherein
r, and s, each independently, is 0, 1, 2, 3, or 4;

3. The tablet composition of claim 1, wherein the compound is represented by Formula (III): 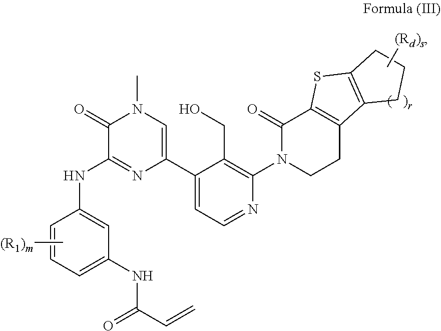
wherein
r, and s, each independently, is 0, 1, 2, 3, or 4;

4. The tablet composition of claim 1, wherein the compound is selected from the group consisting of
(S)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(2-methyl-4-(oxetan-3-yl)piperazin-1-yl)phenyl)acrylamide,
(S)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(2-methyl-4-(tetrahydro-2H-pyran-4-yl)piperazin-1-yl)phenyl)acrylamide,
(R)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(4-(oxetan-3-yl)-2-(trifluoromethyl)piperazin-1-yl)phenyl)acrylamide,
(S)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(4-(oxetan-3-yl)-2-(trifluoromethyl)piperazin-1-yl)phenyl)acrylamide,
(R)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(4-(tetrahydro-2H-pyran-4-yl)-2-(trifluoromethyl)piperazin-1-yl)phenyl)acrylamide,
(S)—N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(4-(tetrahydro-2H-pyran-4-yl)-2-(trifluoromethyl)piperazin-1-yl)phenyl)acrylamide,
(S)—N-(5-((6-(3-(hydroxymethyl)-2-(1-oxo-3,4,5,6,7,8-hexahydrobenzo[4,5]thieno[2,3-c]pyridin-2(1H)-yl)pyridin-4-yl)-4-methyl-3-oxo-3,4-dihydropyrazin-2-yl)amino)-2-(2-methyl-4-(oxetan-3-yl)piperazin-1-yl)phenyl)acrylamide,
(S)—N-(5-((6-(3-(hydroxymethyl)-2-(1-oxo-3,4,5,6,7,8-hexahydrobenzo[4,5]thieno[2,3-c]pyridin-2(1H)-yl)pyridin-4-yl)-4-methyl-3-oxo-3,4-dihydropyrazin-2-yl)amino)-2-(2-methyl-4-(tetrahydro-2H-pyran-4-yl)piperazin-1-yl)phenyl)acrylamide,
(R)—N-(5-((6-(3-(hydroxymethyl)-2-(1-oxo-3,4,5,6,7,8-hexahydrobenzo[4,5]thieno[2,3-c]pyridin-2(1H)-yl)pyridin-4-yl)-4-methyl-3-oxo-3,4-dihydropyrazin-2-yl)amino)-2-(4-(oxetan-3-yl)-2-(trifluoromethyl)piperazin-1-yl)phenyl)acrylamide,
(S)—N-(5-((6-(3-(hydroxymethyl)-2-(1-oxo-3,4,5,6,7,8-hexahydrobenzo[4,5]thieno[2,3-c]pyridin-2(1H)-yl)pyridin-4-yl)-4-methyl-3-oxo-3,4-dihydropyrazin-2-yl)amino)-2-(4-(oxetan-3-yl)-2-(trifluoromethyl)piperazin-1-yl)phenyl)acrylamide,
(R)—N-(5-((6-(3-(hydroxymethyl)-2-(1-oxo-3,4,5,6,7,8-hexahydrobenzo[4,5]thieno[2,3-c]pyridin-2(1H)-yl)pyridin-4-yl)-4-methyl-3-oxo-3,4-dihydropyrazin-2-yl)amino)-2-(4-(tetrahydro-2H-pyran-4-yl)-2-(trifluoromethyl)piperazin-1-yl)phenyl)acrylamide,
(S)—N-(5-((6-(3-(hydroxymethyl)-2-(1-oxo-3,4,5,6,7,8-hexahydrobenzo[4,5]thieno[2,3-c]pyridin-2(1H)-yl)pyridin-4-yl)-4-methyl-3-oxo-3,4-dihydropyrazin-2-yl)amino)-2-(4-(tetrahydro-2H-pyran-4-yl)-2-(trifluoromethyl)piperazin-1-yl)phenyl)acrylamide.

5. The tablet composition of any one of claims 1 to 4, wherein the organic acid is citric acid, fumaric acid, maleic acid, acetic acid, succinic acid, or tartaric acid.

6. The tablet composition of claim 5, wherein the organic acid is fumaric acid.

7. The tablet composition of claim 6, wherein the weight ratio of the compound of Formula (I) to the fumaric acid is from about 1:5 to about 5:1, is from about 1:4 to about 4:1, from about 1:3 to about 3:1, from about 1:2 to about 2:1, from about 1: 1.5 to about 1.5: 1, about 1:1, about 1:1.1, about 1:1.2, about 1:1.25, about 1:1.3, about 1:1.4, or about 1:1.5.

8. The tablet composition of any one of claims 1 to 7, wherein the compound of Formula (I) free base content in the tablet is from about 5 mg to about 500 mg, from about 10 mg to about 250 mg, from about 20 mg to about 100 mg.

9. The tablet composition of any one of claims 1 to 8, wherein the fumaric acid content in the tablet composition is from about 5 wt. % to about 50 wt. %, from about 5 wt. % to about 40 wt. %, from about 5 wt. % to about 30 wt. %, from about 10 wt. % to about 30 wt. %, from about 20 wt. % to about 25 wt. %, from about 5 wt. % to about 15 wt. %, or from about 10 wt. % to about 15 wt. %.

10. The tablet composition of any one of claims 1 to 9, wherein the tablet weight is about 50 mg, about 100 mg, about 200 mg, about 300 mg, about 400 mg, about 500 mg, about 600 mg, about 700 mg, about 800 mg, about 900 mg, about 1000 mg, or about 1100 mg, or about 1200 mg.

11. The tablet composition of any one of claims 1 to 10, further comprising lactose and microcrystalline cellulose.

12. The tablet composition of any one of claims 1 to 11, further comprising at least one pharmaceutically acceptable excipient selected from fillers, binders, disintegrants, lubricants and glidants.

13. A method of treating a neoplastic disease, autoimmune disease, and inflammatory disorder, comprising administering to a subject in need thereof an effective amount of a tablet composition of any one of claims 1 to 12.
